(19) United States
(12) Patent Application Publication
(10) Pub. No.: US 2013/0196971 A1
(43) Pub. Date: Aug. 1, 2013

(54) FATTY ACID SYNTHASE INHIBITORS

(71) Applicants: Christopher Joseph Aquino, Research Triangle Park, NC (US); Dennis Heyer, Durham, NC (US); Terence John Kiesow, Collegeville, PA (US); David Lee Musso, Raleigh, NC (US); Cynthia A. Parrish, Collegeville, PA (US)

(72) Inventors: Christopher Joseph Aquino, Research Triangle Park, NC (US); Dennis Heyer, Durham, NC (US); Terence John Kiesow, Collegeville, PA (US); David Lee Musso, Raleigh, NC (US); Cynthia A. Parrish, Collegeville, PA (US)

(21) Appl. No.: 13/823,931

(22) PCT Filed: Sep. 15, 2011
(86) PCT No.: PCT/US11/51671

§ 371 Date: Mar. 15, 2013

Related U.S. Application Data
(60) Provisional application No. 61/383,771, filed on Sep. 17, 2010.

(51) Int. Cl. C07D 403/06; C07D 471/04; C07D 401/14; C07D 403/14; C07D 405/14

(52) U.S. Cl. CPC C07D 403/06; C07D 403/14; C07D 405/14; C07D 401/14; C07D 471/04
USPC 514/210.18; 548/253; 514/381; 514/382; 548/255; 514/414; 514/406; 514/422; 548/266.4; 514/383; 548/269.4; 548/312.1; 514/397; 548/314.7; 546/272.7; 514/341; 546/113; 514/300; 546/121; 546/173

(57) ABSTRACT

This invention relates to the use of imidazole, triazole, and tetrazole derivatives for the modulation, notably the inhibition of the activity or function of fatty acid synthase (FAS). Suitably, the present invention relates to the use of imidazoles, triazoles, and tetrazoles in the treatment of cancer.

### FIELD OF INVENTION

This invention relates to novel imidazoles, triazoles, and tetrazoles which are inhibitors of fatty acid synthase (FAS), to pharmaceutical compositions containing them, to processes for their preparation, and to their use in therapy for the treatment of cancers.

### BACKGROUND

Fatty acids have an essential role in a variety of cellular processes including building blocks for membranes, anchors for targeting membrane proteins, precursors in the synthesis of lipid second messengers and as a medium to store energy, Menendez J S and Lupu R, Fatty acid synthase and the lipogenic phenotype in cancer pathogenesis, Nature Reviews Cancer, 7: 763-777 (2007). Fatty acids can either be obtained from the diet or can be synthesized de novo from carbohydrate precursors. The biosynthesis of the latter is catalyzed by the muliti-functional homodimeric FAS. FAS synthesizes long chain fatty acids by using acetyl-CoA as a primer and Malonyl Co-A as a 2 carbon donor, and NADPH as reducing equivalents (Wakil S J, Lipids, Structure and function of animal fatty acid synthase, 39: 1045-1053 (2004), Asturias F J et al., Structure and molecular organization of mammalian fatty acid synthase, Nature Struct. Mol. Biol. 12:225-232 (2005), Maier T, et al., Architecture of Mammalian Fatty Acid Synthase at 4.5 Å Resolution, Science 311:1258-1262 (2006)).

De novo fatty acid synthesis is active during embryogenesis and in fetal lungs where fatty acids are used for the production of lung surfactant. In adults, most normal human tissues preferentially acquire fatty acids from the diet. Therefore the level of de novo lipogensis and expression of liopogenic enzymes is low (Weiss L, et al., Fatty-acid biosynthesis in man, a pathway of minor importance. Purification, optimal assay conditions, and organ distribution of fatty-acid synthase. Biological Chemistry Hoppe-Seyler 367(9):905-912 (1986)). In contrast, many tumors have high rates of de novo fatty acid synthesis (Medes G, et al., Metabolism of Neoplastic Tissue. IV. A Study of Lipid Synthesis in Neoplastic Tissue Slices in Vitro, Can Res, 13:27-29, (1953)). FAS has now been shown to be overexpressed in numerous cancer types including prostate, ovary, colon, endometrium lung, bladder, stomach and kidney (Kuhajda F P, Fatty-acid synthase and human cancer: new perspectives on its role in tumor biology, Nutrition; 16:202-208 (2000)). This differential expression and function of FAS in tumors and normal cells provide an approach for cancer therapy with the potential of a substantial therapeutic window.

Pharmacological and small interference RNA mediated inhibition of FAS has demonstrated a preferential inhibition of cancer cell proliferation. Additionally these inhibitors induce apoptosis in cancers cells in vitro and retard growth in human tumors in murine xenograft models in vivo (Menendez J S and Lupu R, Nature Reviews Cancer, 7: 763-777 (2007)). Based upon these findings, FAS is considered a major potential target of antineoplastic intervention.

### SUMMARY OF THE INVENTION

This invention relates to compounds of the Formula (I), as shown below:

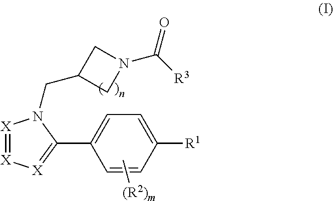

wherein:

R¹ is phenyl, naphthyl, 5- or 6-membered heteroaryl, or 9- or 10-membered heterocyclyl; wherein said phenyl, naphthyl, 5- or 6-membered heteroaryl, or 9- or 10-membered heterocyclyl is optionally substituted 1 to 3 times independently by halogen, (C₁-C₄)alkyl, —CF₃, (C₃-C₇)cycloalkyl, —CO(C₁-C₄)alkyl, —CO(C₃-C₇)cycloalkyl, —CO(phenyl), carboxyl, —CO₂(C₁-C₄)alkyl, —CONR⁵R⁶, phenyl, —SO₂(C₁-C₄)alkyl, —SO₂NR⁵R⁶, cyano, oxo, hydroxyl, (C₁-C₄)alkoxy, (C₃-C₇)cycloalkoxy, hydroxy(C₁-C₄)alkyl-, (C₁-C₄)alkoxy(C₁-C₄)alkyl-, —OCF₃, —NR⁵R⁶, R⁵R⁶N(C₁-C₄)alkyl-, —NHCO(C₁-C₄)alkyl, —NHCONR⁵R⁶, —NHSO₂(C₁-C₄)alkyl, —NHSO₂NR⁵R⁶, or R⁹;

when present each R² is independently selected from the group consisting of halogen, (C₁-C₆)alkyl, hydroxyl, and (C₁-C₄)alkoxy;

R³ is selected from the group consisting of (C₁-C₆)alkyl, —CF₃, (C₃-C₇)cycloalkyl, (C₁-C₄)alkoxy, and —NR⁷R⁸; wherein said (C₁-C₆)alkyl is optionally substituted by hydroxyl, (C₁-C₄)alkoxy, —CF₃, or cyano, and wherein said (C₃-C₇)cycloalkyl is optionally substituted 1 or 2 times independently by halogen, (C₁-C₄)alkyl, hydroxy(C₁-C₄)alkyl-, (C₁-C₄)alkoxy(C₁-C₄)alkyl-, —CF₃, or cyano;

each X is independently N or CR⁴, wherein at least one X is N;

when present each R⁴ is independently hydrogen or (C₁-C₄)alkyl;

R⁵ is selected from the group consisting of hydrogen, (C₁-C₄)alkyl, (C₃-C₇)cycloalkyl, phenyl, and phenyl(C₁-C₃)alkyl-;

R⁶ is hydrogen, (C₁-C₄)alkyl, or (C₃-C₇)cycloalkyl;

or R⁵ and R⁶ taken together with the nitrogen to which they are attached represent a 3- to 7-membered saturated ring optionally containing one other heteroatom which is oxygen, nitrogen, or sulfur, which ring is optionally substituted 1 or 2 times independently by oxo or (C₁-C₄)alkyl;

R⁷ and R⁸ are each independently hydrogen, (C₁-C₄)alkyl, or (C₃-C₇)cycloalkyl;

or R⁷ and R⁸ taken together with the nitrogen to which they are attached represent a 3- to 7-membered saturated ring optionally containing one other heteroatom which is oxygen, nitrogen, or sulfur, which ring is optionally substituted 1 or 2 times independently by oxo or (C₁-C₄)alkyl;

R⁹ is a 5-membered heteroaryl ring containing 1 to 4 heteroatoms selected from oxygen, nitrogen, and sulfur, or a 6-membered heteroaryl ring containing 1 to 3 nitrogen atoms, which 5- or 6-membered ring is optionally substituted 1 or 2 times independently by halogen, (C₁-C₄)alkyl, —CF₃, (C₁-C₄)alkoxy, or —NR⁵R⁶;

m is 0-3; and

n is 1 or 2;

or pharmaceutically acceptable salts thereof.

This invention also relates to pharmaceutical compositions, which comprise compounds of Formula (I) and pharmaceutically acceptable carriers.

This invention also relates to methods of treating cancer which comprise administering an effective amount of a compound of Formula (I) to a human in need thereof.

This invention also relates to methods of treating cancer which comprise co-administering an compound of Formula (I) and a second compound to a human in need thereof.

### DETAILED DESCRIPTION OF THE INVENTION

This invention relates to compounds of Formula (I), and pharmaceutically acceptable salts thereof.

This invention also relates to compounds of Formula (I)(A):

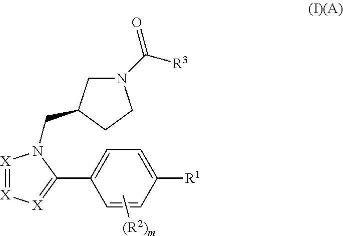

or pharmaceutically acceptable salts thereof, wherein R¹, R², R³, X, and m are defined according to Formula (I).

This invention also relates to compounds of Formula (I)(B):

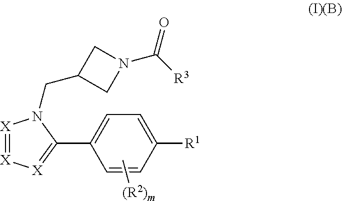

or pharmaceutically acceptable salts thereof, wherein R¹, R², R³, X, and m are defined according to Formula (I).

In one embodiment, this invention relates to compounds of Formula (I), (I)(A), or (I)(B), wherein R¹ is phenyl which is optionally substituted 1 to 3 times independently by halogen, (C₁-C₄)alkyl, —CF₃, (C₃-C₇)cycloalkyl, —CO(C₁-C₄)alkyl, —CO(C₃-C₇)cycloalkyl, —CO(phenyl), carboxyl, —CO₂(C₁-C₄)alkyl, —CONR⁵R⁶, phenyl, —SO₂(C₁-C₄)alkyl, —SO₂NR⁵R⁶, cyano, oxo, hydroxyl, (C₁-C₄)alkoxy, (C₃-C₇)cycloalkoxy, hydroxy(C₁-C₄)alkyl-, (C₁-C₄)alkoxy(C₁-C₄)alkyl-, —OCF₃, —NR⁵R⁶, R⁵R⁶N(C₁-C₄)alkyl-, —NHCO(C₁-C₄)alkyl, —NHCONR⁵R⁶, —NHSO₂(C₁-C₄)alkyl, —NHSO₂NR⁵R⁶, or R⁹, or pharmaceutically acceptable salts thereof.

In another embodiment, this invention also relates to compounds of Formula (I), (I)(A), or (I)(B), wherein R¹ is phenyl, 4-fluorophenyl, 3-chlorophenyl, 4-chlorophenyl, 2,4-difluorophenyl, 3,4-difluorophenyl, 3-chloro-4-fluorophenyl, 2,4-dichlorophenyl, 2-fluoro-4-methylphenyl, 3-fluoro-4-methylphenyl, 4-fluoro-3-methylphenyl, 2-fluoro-4-methoxyphenyl, 3-fluoro-4-methoxyphenyl, 4-fluoro-3-hydroxyphenyl, 4-fluoro-3-methoxyphenyl, 2-chloro-4-methoxyphenyl, 3-chloro-4-methoxyphenyl, 3-methylphenyl, 4-methylphenyl, 2,4-dimethylphenyl, 2-cyanophenyl, 4-cyanophenyl, 3-hydroxyphenyl, 4-hydroxyphenyl, 4-methoxyphenyl, 4-ethoxyphenyl, 3-hydroxy-4-methylphenyl, 3-methoxy-4-methylphenyl, 4-methoxy-3-methylphenyl, 3-hydroxy-4-methoxyphenyl, 4-(dimethylamino)phenyl, 3-{[(dimethylamino)sulfonyl]amino}phenyl, 4-(1H-pyrazol-1-yl)phenyl, 4-(1H-pyrazol-5-yl)phenyl, or 3-(1H-tetrazol-5-yl)phenyl, or pharmaceutically acceptable salts thereof.

In another embodiment, this invention also relates to compounds of Formula (I), (I)(A), or (I)(B), wherein R¹ is 5- or 6-membered heteroaryl which is optionally substituted 1 to 3 times independently by halogen, (C₁-C₄)alkyl, —CF₃, (C₃-C₇)cycloalkyl, —CO(C₁-C₄)alkyl, —CO(C₃-C₇)cycloalkyl, —CO(phenyl), carboxyl, —CO₂(C₁-C₄)alkyl, —CONR⁵R⁶, phenyl, —SO₂(C₁-C₄)alkyl, —SO₂NR⁵R⁶, cyano, oxo, hydroxyl, (C₁-C₄)alkoxy, (C₃-C₇)cycloalkoxy, hydroxy(C₁-C₄)alkyl-, (C₁-C₄)alkoxy(C₁-C₄)alkyl-, —OCF₃, —NR⁵R⁶, R⁵R⁶N(C₁-C₄)alkyl-, —NHCO(C₁-C₄)alkyl, —NHCONR⁵R⁶, —NHSO₂(C₁-C₄)alkyl, —NHSO₂NR⁵R⁶, or R⁹, or pharmaceutically acceptable salts thereof.

In another embodiment, this invention also relates to compounds of Formula (I), (I)(A), or (I)(B), wherein R¹ is furanyl, thienyl, pyrrolyl, imidazolyl, pyrazolyl, triazolyl, tetrazolyl, thiazolyl, oxazolyl, isoxazolyl, oxadiazolyl, thiadiazolyl, isothiazolyl, pyridinyl, pyridazinyl, pyrazinyl, pyrimidinyl, or triazinyl, wherein said furanyl, thienyl, pyrrolyl, imidazolyl, pyrazolyl, triazolyl, tetrazolyl, thiazolyl, oxazolyl, isoxazolyl, oxadiazolyl, thiadiazolyl, isothiazolyl, pyridinyl, pyridazinyl, pyrazinyl, pyrimidinyl, or triazinyl is optionally substituted 1 to 3 times independently by halogen, (C₁-C₄)alkyl, —CF₃, (C₃-C₇)cycloalkyl, —CO(C₁-C₄)alkyl, —CO(C₃-C₇)cycloalkyl, —CO(phenyl), carboxyl, —CO₂(C₁-C₄)alkyl, —CONR⁵R⁶, phenyl, —SO₂(C₁-C₄)alkyl, —SO₂NR⁵R⁶, cyano, oxo, hydroxyl, (C₁-C₄)alkoxy, (C₃-C₇)cycloalkoxy, hydroxy(C₁-C₄)alkyl-, (C₁-C₄)alkoxy(C₁-C₄)alkyl-, —OCF₃, —NR⁵R⁶, R⁵R⁶N(C₁-C₄)alkyl-, —NHCO(C₁-C₄)alkyl, —NHCONR⁵R⁶, —NHSO₂(C₁-C₄)alkyl, —NHSO₂NR⁵R⁶, or R⁹, or pharmaceutically acceptable salts thereof.

In another embodiment, this invention also relates to compounds of Formula (I), (I)(A), or (I)(B), wherein R¹ is pyridin-3-yl, or pharmaceutically acceptable salts thereof.

In another embodiment, this invention also relates to compounds of Formula (I), (I)(A), or (I)(B), wherein R¹ is 9- or 10-membered heterocyclyl which is optionally substituted 1 to 3 times independently by halogen, (C₁-C₄)alkyl, —CF₃, (C₃-C₇)cycloalkyl, —CO(C₁-C₄)alkyl, —CO(C₃-C₇)cycloalkyl, —CO(phenyl), carboxyl, —CO₂(C₁-C₄)alkyl, —CONR⁵R⁶, phenyl, —SO₂(C₁-C₄)alkyl, —SO₂NR⁵R⁶, cyano, oxo, hydroxyl, (C₁-C₄)alkoxy, (C₃-C₇)cycloalkoxy, hydroxy(C₁-C₄)alkyl-, (C₁-C₄)alkoxy(C₁-C₄)alkyl-, —OCF₃, —NR⁵R⁶, R⁵R⁶N(C₁-C₄)alkyl-, —NHCO(C₁-C₄)alkyl, —NHCONR⁵R⁶, —NHSO₂(C₁-C₄)alkyl, —NHSO₂NR⁵R⁶, or R⁹, or pharmaceutically acceptable salts thereof.

In another embodiment, this invention also relates to compounds of Formula (I), (I)(A), or (I)(B), wherein R¹ is benzofuranyl, isobenzofuryl, 2,3-dihydrobenzofuryl, 1,3-benzodioxolyl, dihydrobenzodioxinyl, benzothienyl, indolizinyl, indolyl, isoindolyl, indolinyl, isoindolinyl, benzimidazolyl, dihydrobenzimidazolyl, benzoxazolyl, dihydrobenzoxazolyl, benzthiazolyl, benzoisothiazolyl, dihydrobenzoisothiazolyl, indazolyl, pyrrolopyridinyl, pyrrolopyrimidinyl, imidazopyridinyl, imidazopyrimidinyl, pyrazolopyridinyl, pyrazolopyrimidinyl, benzoxadiazolyl, benzthiadiazolyl, benzotriazolyl, triazolopyridinyl, purinyl, quinolinyl, tetrahydroquinolinyl, isoquinolinyl, tetrahydroisoquinolinyl, quinoxalinyl, cinnolinyl, phthalazinyl, quinazolinyl, 1,5-naphthyridinyl, 1,6-naphthyridinyl, 1,7-naphthyridinyl, 1,8-naphthyridinyl, or pteridinyl, wherein said benzofuranyl, isobenzofuryl, 2,3-dihydrobenzofuryl, 1,3-benzodioxolyl, dihydrobenzodioxinyl, benzothienyl, indolizinyl, indolyl, isoindolyl, indolinyl, isoindolinyl, benzimidazolyl, dihydrobenzimidazolyl, benzoxazolyl, dihydrobenzoxazolyl, benzthiazolyl, benzoisothiazolyl, dihydrobenzoisothiazolyl, indazolyl, pyrrolopyridinyl, pyrrolopyrimidinyl, imidazopyridinyl, imidazopyrimidinyl, pyrazolopyridinyl, pyrazolopyrimidinyl, benzoxadiazolyl, benzthiadiazolyl, benzotriazolyl, triazolopyridinyl, purinyl, quinolinyl, tetrahydroquinolinyl, isoquinolinyl, tetrahydroisoquinolinyl, quinoxalinyl, cinnolinyl, phthalazinyl, quinazolinyl, 1,5-naphthyridinyl, 1,6-naphthyridinyl, 1,7-naphthyridinyl, 1,8-naphthyridinyl, or pteridinyl is optionally substituted 1 to 3 times independently by halogen, (C₁-C₄)alkyl, —CF₃, (C₃-C₇)cycloalkyl, —CO(C₁-C₄)alkyl, —CO(C₃-C₇)cycloalkyl, —CO(phenyl), carboxyl, —CO₂(C₁-C₄)alkyl, —CONR⁵R⁶, phenyl, —SO₂(C₁-C₄)alkyl, —SO₂NR⁵R⁶, cyano, oxo, hydroxyl, (C₁-C₄)alkoxy, (C₃-C₇)cycloalkoxy, hydroxy(C₁-C₄)alkyl-, (C₁-C₄)alkoxy(C₁-C₄)alkyl-, —OCF₃, —NR⁵R⁶, R⁵R⁶N(C₁-C₄)alkyl-, —NHCO(C₁-C₄)alkyl, —NHCONR⁵R⁶, —NHSO₂(C₁-C₄)alkyl, —NHSO₂NR⁵R⁶, or R⁹, or pharmaceutically acceptable salts thereof.

In another embodiment, this invention also relates to compounds of Formula (I), (I)(A), or (I)(B), wherein R¹ is benzofuranyl, 2,3-dihydrobenzofuryl, indolyl, indolinyl, benzthiazolyl, benzimidazolyl, benzoxazolyl, indazolyl, pyrrolopyridinyl, imidazopyridinyl, quinolinyl, or isoquinolinyl, wherein said benzofuranyl, 2,3-dihydrobenzofuryl, indolyl, indolinyl, benzthiazolyl, benzimidazolyl, benzoxazolyl, indazolyl, pyrrolopyridinyl, imidazopyridinyl, quinolinyl, or isoquinolinyl is optionally substituted 1 to 3 times independently by halogen, (C₁-C₄)alkyl, —CF₃, (C₃-C₇)cycloalkyl, —CO(C₁-C₄)alkyl, —CO(C₃-C₇)cycloalkyl, —CO(phenyl), carboxyl, —CO₂(C₁-C₄)alkyl, —CONR⁵R⁶, phenyl, —SO₂(C₁-C₄)alkyl, —SO₂NR⁵R⁶, cyano, oxo, hydroxyl, (C₁-C₄)alkoxy, (C₃-C₇)cycloalkoxy, hydroxy(C₁-C₄)alkyl-, (C₁-C₄)alkoxy(C₁-C₄)alkyl-, —OCF₃, —NR⁵R⁶, R⁵R⁶N(C₁-C₄)alkyl-, —NHCO(C₁-C₄)alkyl, —NHCONR⁵R⁶, —NHSO₂(C₁-C₄)alkyl, —NHSO₂NR⁵R⁶, or R⁹, or pharmaceutically acceptable salts thereof.

In another embodiment, this invention also relates to compounds of Formula (I), (I)(A), or (I)(B), wherein R¹ is benzofuranyl, 2,3-dihydrobenzofuryl, indolyl, indolinyl, benzthiazolyl, indazolyl, pyrrolopyridinyl, imidazopyridinyl, or quinolinyl, wherein said benzofuranyl, 2,3-dihydrobenzofuryl, indolyl, indolinyl, benzthiazolyl, indazolyl, pyrrolopyridinyl, imidazopyridinyl, or quinolinyl is optionally substituted by (C₁-C₄)alkyl, —CF₃, cyano, hydroxyl, methoxy, —OCF₃, amino, methylamino or dimethylamino, or pharmaceutically acceptable salts thereof.

In another embodiment, this invention also relates to compounds of Formula (I), (I)(A), or (I)(B), wherein R¹ is benzofuran-5-yl, 2,3-dihydro-1-benzofuran-5-yl, 1H-indol-4-yl, 1H-indol-5-yl, 1H-indol-6-yl, 1-methyl-1H-indole-5-yl, 1H-indazol-4-yl, 1H-indazol-5-yl, 1H-indazol-6-yl, 2,3-dihydro-1H-indol-5-yl, 1,3-benzothiazol-6-yl, imidazo[1,2-a]pyridin-7-yl, 1H-pyrrolo[3,2-b]pyridin-6-yl, 1-methyl-1H-pyrrolo[2,3-b]pyridin-5-yl, quinolin-3-yl, quinolin-6-yl, or quinolin-7-yl, or pharmaceutically acceptable salts thereof.

In another embodiment, this invention also relates to compounds of any of the above embodiments, wherein R² is fluoro, chloro, hydroxyl, methoxy, or methyl, and m is 1, or pharmaceutically acceptable salts thereof.

In another embodiment, this invention also relates to compounds of any of the above embodiments, wherein R³ is (C₁-C₄)alkyl, —CF₃, (C₃-C₆)cycloalkyl, methoxy, or dimethylamino, wherein said (C₃-C₆)cycloalkyl is optionally substituted 1 or 2 times independently by fluoro or methyl, or pharmaceutically acceptable salts thereof.

In another embodiment, this invention also relates to compounds of any of the above embodiments, wherein R³ is methyl, ethyl, isopropyl, t-butyl, —CF₃, cyclopropyl, 1-methyl-cyclopropyl, 2,2-difluoro-cyclopropyl, cyclopentyl, methoxy, or dimethylamino, or pharmaceutically acceptable salts thereof.

In another embodiment, this invention also relates to compounds of any of the above embodiments, wherein R³ is cyclopropyl, or pharmaceutically acceptable salts thereof.

In another embodiment, this invention also relates to compounds of any of the above embodiments, wherein R⁴ is hydrogen or methyl, or pharmaceutically acceptable salts thereof.

One particular embodiment of the invention is a compound of Formula (I) wherein:

R¹ is phenyl, 5- or 6-membered heteroaryl, or 9- or 10-membered heterocyclyl; wherein said phenyl, 5- or 6-membered heteroaryl, or 9- or 10-membered heterocyclyl is optionally substituted 1 to 3 times independently by halogen, (C₁-C₄)alkyl, —CF₃, (C₃-C₇)cycloalkyl, —CO(C₁-C₄)alkyl, —CO(C₃-C₇)cycloalkyl, —CO(phenyl), carboxyl, —CO₂(C₁-C₄)alkyl, —CONR⁵R⁶, phenyl, —SO₂(C₁-C₄)alkyl, —SO₂NR⁵R⁶, cyano, oxo, hydroxyl, (C₁-C₄)alkoxy, (C₃-C₇)cycloalkoxy, hydroxy(C₁-C₄)alkyl-, (C₁-C₄)alkoxy(C₁-C₄)alkyl-, —OCF₃, —NR⁵R⁶, R⁵R⁶N(C₁-C₄)alkyl-, —NHCO(C₁-C₄)alkyl, —NHCONR⁵R⁶, —NHSO₂(C₁-C₄)alkyl, —NHSO₂NR⁵R⁶, or R⁹; when present each R² is independently selected from the group consisting of halogen, (C₁-C₆)alkyl, hydroxyl, and (C₁-C₄)alkoxy;

R³ is selected from the group consisting of (C₁-C₆)alkyl, —CF₃, (C₃-C₇)cycloalkyl, (C₁-C₄)alkoxy, and —NR⁷R⁸; wherein said (C₃-C₇)cycloalkyl is optionally substituted 1 or 2 times independently by halogen or (C₁-C₄)alkyl;

each X is independently N or CR⁴, wherein at least one X is N;

when present each R⁴ is independently hydrogen or (C₁-C₄)alkyl;

R⁵ is selected from the group consisting of hydrogen, (C₁-C₄)alkyl, phenyl, and phenyl(C₁-C₃)alkyl-;

R⁶ is hydrogen or (C₁-C₄)alkyl;

or R⁵ and R⁶ taken together with the nitrogen to which they are attached represent a 3- to 7-membered saturated ring optionally containing one other heteroatom which is oxygen, nitrogen, or sulfur, which is optionally substituted 1 or 2 times independently by oxo or (C₁-C₄)alkyl;

R⁷ and R⁸ are each independently hydrogen or (C₁-C₄)alkyl;

or R⁷ and R⁸ taken together with the nitrogen to which they are attached represent a 3- to 7-membered saturated ring optionally containing one other heteroatom which is oxygen, nitrogen, or sulfur;

R⁹ is a 5-membered heteroaryl ring containing 1 to 4 heteroatoms selected from oxygen, nitrogen, and sulfur, which is optionally substituted 1 or 2 times independently by halogen, (C₁-C₄)alkyl, (C₁-C₄)alkoxy, or —NR⁵R⁶;

m is 0-3; and

n is 1 or 2;

or pharmaceutically acceptable salts thereof.

Another particular embodiment of the invention is a compound of Formula (I)(A) wherein:

R¹ is phenyl, 5- or 6-membered heteroaryl, or 9- or 10-membered heterocyclyl; wherein said phenyl, 5- or 6-membered heteroaryl, or 9- or 10-membered heterocyclyl is optionally substituted 1 to 3 times independently by halogen, (C₁-C₄)alkyl, —CF₃, (C₃-C₇)cycloalkyl, —CO(C₁-C₄)alkyl, —CO(C₃-C₇)cycloalkyl, —CO(phenyl), carboxyl, CO₂(C₁-C₄)alkyl, —CONR⁵R⁶, phenyl, —SO₂(C₁-C₄)alkyl, —SO₂NR⁵R⁶, cyano, oxo, hydroxyl, (C₁-C₄)alkoxy, (C₃-C₇)cycloalkoxy, hydroxy(C₁-C₄)alkyl-, (C₁-C₄)alkoxy(C₁-C₄)alkyl-, —OCF₃, —NR⁵R⁶, R⁵R⁶N(C₁-C₄)alkyl-, —NHCO(C₁-C₄)alkyl, —NHCONR⁵R⁶, —NHSO₂(C₁-C₄)alkyl, —NHSO₂NR⁵R⁶, or R⁹;

when present each R² is independently selected from the group consisting of halogen, (C₁-C₆)alkyl, hydroxyl, and (C₁-C₄)alkoxy;

R³ is selected from the group consisting of (C₁-C₆)alkyl, —CF₃, (C₃-C₇)cycloalkyl, (C₁-C₄)alkoxy, and —NR⁷R⁸; wherein said (C₃-C₇)cycloalkyl is optionally substituted 1 or 2 times independently by halogen or (C₁-C₄)alkyl;

each X is independently N or CR⁴, wherein at least one X is N;

when present each R⁴ is independently hydrogen or (C₁-C₄)alkyl;

R⁵ is selected from the group consisting of hydrogen, (C₁-C₄)alkyl, phenyl, and phenyl(C₁-C₃)alkyl-;

R⁶ is hydrogen or (C₁-C₄)alkyl;

or R⁵ and R⁶ taken together with the nitrogen to which they are attached represent a 3- to 7-membered saturated ring optionally containing one other heteroatom which is oxygen, nitrogen, or sulfur, which is optionally substituted 1 or 2 times independently by oxo or (C₁-C₄)alkyl;

R⁷ and R⁸ are each independently hydrogen or (C₁-C₄)alkyl;

or R⁷ and R⁸ taken together with the nitrogen to which they are attached represent a 3- to 7-membered saturated ring optionally containing one other heteroatom which is oxygen, nitrogen, or sulfur;

R⁹ is a 5-membered heteroaryl ring containing 1 to 4 heteroatoms selected from oxygen, nitrogen, and sulfur, which is optionally substituted 1 or 2 times independently by halogen, (C₁-C₄)alkyl, (C₁-C₄)alkoxy, or —NR⁵R⁶; and

m is 0-3;

or pharmaceutically acceptable salts thereof.

Another particular embodiment of the invention is a compound of Formula (I)(B) wherein:

R¹ is phenyl, 5- or 6-membered heteroaryl, or 9- or 10-membered heterocyclyl; wherein said phenyl, 5- or 6-membered heteroaryl, or 9- or 10-membered heterocyclyl is optionally substituted 1 to 3 times independently by halogen, (C₁-C₄)alkyl, —CF₃, (C₃-C₇)cycloalkyl, —CO(C₁-C₄)alkyl, —CO(C₃-C₇)cycloalkyl, —CO(phenyl), carboxyl, —CO₂(C₁-C₄)alkyl, —CONR⁵R⁶, phenyl, —SO₂(C₁-C₄)alkyl, —SO₂NR⁵R⁶, cyano, oxo, hydroxyl, (C₁-C₄)alkoxy, (C₃-C₇)cycloalkoxy, hydroxy(C₁-C₄)alkyl-, (C₁-C₄)alkoxy(C₁-C₄)alkyl-, —OCF₃, —NR⁵R⁶, R⁵R⁶N(C₁-C₄)alkyl-, —NHCO(C₁-C₄)alkyl, —NHCONR⁵R⁶, —NHSO₂(C₁-C₄)alkyl, —NHSO₂NR⁵R⁶, or R⁹;

when present each R² is independently selected from the group consisting of halogen, (C₁-C₆)alkyl, hydroxyl, and (C₁-C₄)alkoxy;

R³ is selected from the group consisting of (C₁-C₆)alkyl, —CF₃, (C₃-C₇)cycloalkyl, (C₁-C₄)alkoxy, and —NR⁷R⁸; wherein said (C₃-C₇)cycloalkyl is optionally substituted 1 or 2 times independently by halogen or (C₁-C₄)alkyl;

each X is independently N or CR⁴, wherein at least one X is N; when present each R⁴ is independently hydrogen or (C₁-C₄)alkyl;

R⁵ is selected from the group consisting of hydrogen, (C₁-C₄)alkyl, phenyl, and phenyl(C₁-C₃)alkyl-;

R⁶ is hydrogen or (C₁-C₄)alkyl; or R⁵ and R⁶ taken together with the nitrogen to which they are attached represent a 3- to 7-membered saturated ring optionally containing one other heteroatom which is oxygen, nitrogen, or sulfur, which is optionally substituted 1 or 2 times independently by oxo or (C₁-C₄)alkyl;

R⁷ and R⁸ are each independently hydrogen or (C₁-C₄)alkyl;

or R⁷ and R⁸ taken together with the nitrogen to which they are attached represent a 3- to 7-membered saturated ring optionally containing one other heteroatom which is oxygen, nitrogen, or sulfur;

R⁹ is a 5-membered heteroaryl ring containing 1 to 4 heteroatoms selected from oxygen, nitrogen, and sulfur, which is optionally substituted 1 or 2 times independently by halogen, (C₁-C₄)alkyl, (C₁-C₄)alkoxy, or —NR⁵R⁶; and

m is 0-3;

or pharmaceutically acceptable salts thereof.

This invention also relates to the following compounds:
6-[4-(1-{[(3R)-1-(cyclopropylcarbonyl)-3-pyrrolidinyl]methyl}-1H-tetrazol-5-yl)phenyl]-1H-indole;
5-[4-(1-benzofuran-5-yl)phenyl]-1-{[(3R)-1-(cyclopropylcarbonyl)-3-pyrrolidinyl]methyl}-1H-tetrazole;
5-[4-(1-{[(3R)-1-(cyclopropylcarbonyl)-3-pyrrolidinyl]methyl}-1H-tetrazol-5-yl)phenyl]-1H-indole;
1-{[(3R)-1-(cyclopropylcarbonyl)-3-pyrrolidinyl]methyl}-5-(2′,4′-dichloro-4-biphenylyl)-1H-tetrazole;
5-[2′-chloro-4′-(methyloxy)-4-biphenylyl]-1-{[(3R)-1-(cyclopropylcarbonyl)-3-pyrrolidinyl]methyl}-1H-tetrazole;
1-{[(3R)-1-(cyclopropylcarbonyl)-3-pyrrolidinyl]methyl}-5-(4′-fluoro-4-biphenylyl)-1H-tetrazole;
6-[4-(1-{[(3R)-1-(cyclopropylcarbonyl)-3-pyrrolidinyl]methyl}-1H-tetrazol-5-yl)phenyl]-1H-indazole;
6-[4-(1-{[(3R)-1-(cyclopropylcarbonyl)-3-pyrrolidinyl]methyl}-1H-1,2,3-triazol-5-yl)phenyl]-1H-indole;
5-[4-(1-{[(3R)-1-(cyclopropylcarbonyl)-3-pyrrolidinyl]methyl}-1H-1,2,3-triazol-5-yl)phenyl]-1H-indole;
5-[4-(1-benzofuran-5-yl)phenyl]-1-{[(3R)-1-(cyclopropylcarbonyl)-3-pyrrolidinyl]methyl}-1H-1,2,3-triazole;
5-[4-(1-{[(3R)-1-(cyclopropylcarbonyl)-3-pyrrolidinyl]methyl}-1H-1,2,3-triazol-5-yl)phenyl]-1H-indazole;
1-{[(3R)-1-(cyclopropylcarbonyl)-3-pyrrolidinyl]methyl}-5-(2′,4′-dichloro-4-biphenylyl)-1H-1,2,3-triazole;
5-[2′-chloro-4′-(methyloxy)-4-biphenylyl]-1-{[(3R)-1-(cyclopropylcarbonyl)-3-pyrrolidinyl]methyl}-1H-1,2,3-triazole;
6-[4-(4-{[(3S)-1-(cyclopropylcarbonyl)-3-pyrrolidinyl]methyl}-4H-1,2,4-triazol-3-yl)phenyl]-1H-indole;
4-{[(3S)-1-(cyclopropylcarbonyl)-3-pyrrolidinyl]methyl}-3-(2′,4′-dichloro-4-biphenylyl)-5-methyl-4H-1,2,4-triazole;
6-[4-(4-{[(3S)-1-(cyclopropylcarbonyl)-3-pyrrolidinyl]methyl}-5-methyl-4H-1,2,4-triazol-3-yl)phenyl]-1H-indole;
3-[4-(1-benzofuran-5-yl)phenyl]-4-{[(3S)-1-(cyclopropylcarbonyl)-3-pyrrolidinyl]methyl}-5-methyl-4H-1,2,4-triazole;
4-{[(3S)-1-(cyclopropylcarbonyl)-3-pyrrolidinyl]methyl}-3-(2′,4′-dichloro-4-biphenylyl)-4H-1,2,4-triazole;
5-[4-(4-{[(3S)-1-(cyclopropylcarbonyl)-3-pyrrolidinyl]methyl}-4H-1,2,4-triazol-3-yl)phenyl]-1H-indazole;
3-[2′-chloro-4′-(methyloxy)-4-biphenylyl]-4-{[(3S)-1-(cyclopropylcarbonyl)-3-pyrrolidinyl]methyl}-4H-1,2,4-triazole;
5-[4-(1-{[(3S)-1-(cyclopropylcarbonyl)-3-pyrrolidinyl]methyl}-1H-imidazol-2-yl)phenyl]-1H-indole;
6-[4-(1-{[(3S)-1-(cyclopropylcarbonyl)-3-pyrrolidinyl]methyl}-1H-imidazol-2-yl)phenyl]-1H-indole;
2-(3′-chloro-4-biphenylyl)-1-{[(3S)-1-(cyclopropylcarbonyl)-3-pyrrolidinyl]methyl}-1H-imidazole;
1-{[1-(cyclopropylcarbonyl)-3-pyrrolidinyl]methyl}-2-[4′-(methyloxy)-4-biphenylyl]-1H-imidazole;
1-{[1-(cyclopropylcarbonyl)-3-pyrrolidinyl]methyl}-2-(3′-fluoro-4′-methyl-4-biphenylyl)-1H-imidazole;
2-(4-biphenylyl)-1-{[(3S)-1-(cyclopropylcarbonyl)-3-pyrrolidinyl]methyl}-1H-imidazole;
5-[4-(1-{[1-(cyclopropylcarbonyl)-3-pyrrolidinyl]methyl}-1H-imidazol-2-yl)phenyl]-1H-indole;
2-(3′-chloro-4-biphenylyl)-1-{[1-(cyclopropylcarbonyl)-3-pyrrolidinyl]methyl}-1H-imidazole;
2-(4′-chloro-4-biphenylyl)-1-{[(3S)-1-(cyclopropylcarbonyl)-3-pyrrolidinyl]methyl}-1H-imidazole;
1-{[(3S)-1-(cyclopropylcarbonyl)-3-pyrrolidinyl]methyl}-2-(2′,4′-dichloro-4-biphenylyl)-1H-imidazole;
1-{[(3S)-1-(cyclopropylcarbonyl)-3-pyrrolidinyl]methyl}-2-(4′-fluoro-4-biphenylyl)-1H-imidazole;
3-[4-(1-{[(3S)-1-(cyclopropylcarbonyl)-3-pyrrolidinyl]methyl}-1H-imidazol-2-yl)phenyl]pyridine;
6-[4-(1-{[1-(cyclopropylcarbonyl)-3-pyrrolidinyl]methyl}-1H-imidazol-2-yl)phenyl]-1H-indole;
2-[4-(1-benzofuran-5-yl)phenyl]-1-{[(3S)-1-(cyclopropylcarbonyl)-3-pyrrolidinyl]methyl}-1H-imidazole;
1-{[1-(cyclopropylcarbonyl)-3-pyrrolidinyl]methyl}-4,5-dimethyl-2-[4′-(methyloxy)-4-biphenylyl]-1H-imidazole;
1-{[1-(cyclopropylcarbonyl)-3-pyrrolidinyl]methyl}-2-(2′,4′-dichloro-4-biphenylyl)-4,5-dimethyl-1H-imidazole;
2-[2′-chloro-4′-(methyloxy)-4-biphenylyl]-1-{[1-(cyclopropylcarbonyl)-3-pyrrolidinyl]methyl}-4,5-dimethyl-1H-imidazole;
2-(3′-chloro-4′-fluoro-4-biphenylyl)-1-{[1-(cyclopropylcarbonyl)-3-pyrrolidinyl]methyl}-4,5-dimethyl-1H-imidazole;
1-{[1-(cyclopropylcarbonyl)-3-pyrrolidinyl]methyl}-2-(4′-fluoro-3′-methyl-4-biphenylyl)-4,5-dimethyl-1H-imidazole;
1-{[1-(cyclopropylcarbonyl)-3-pyrrolidinyl]methyl}-4,5-dimethyl-2-(4′-methyl-4-biphenylyl)-1H-imidazole;
1-{[1-(cyclopropylcarbonyl)-3-pyrrolidinyl]methyl}-2-(4′-fluoro-4-biphenylyl)-4,5-dimethyl-1H-imidazole;
4′-(1-{[1-(cyclopropylcarbonyl)-3-pyrrolidinyl]methyl}-4,5-dimethyl-1H-imidazol-2-yl)-3-biphenylol;
1-{[1-(cyclopropylcarbonyl)-3-pyrrolidinyl]methyl}-4,5-dimethyl-2-(3′-methyl-4-biphenylyl)-1H-imidazole;
2-(3′-chloro-4-biphenylyl)-1-{[1-(cyclopropylcarbonyl)-3-pyrrolidinyl]methyl}-4,5-dimethyl-1H-imidazole;
2-(4′-chloro-4-biphenylyl)-1-{[1-(cyclopropylcarbonyl)-3-pyrrolidinyl]methyl}-4,5-dimethyl-1H-imidazole;
1-{[1-(cyclopropylcarbonyl)-3-pyrrolidinyl]methyl}-5-methyl-2-(4′-methyl-4-biphenylyl)-1H-imidazole;
1-{[1-(cyclopropylcarbonyl)-3-pyrrolidinyl]methyl}-2-[4′-(ethyloxy)-4-biphenylyl]-5-methyl-1H-imidazole;
1-{[1-(cyclopropylcarbonyl)-3-pyrrolidinyl]methyl}-5-methyl-2-[4′-(methyloxy)-4-biphenylyl]-1H-imidazole;
2-(4′-chloro-4-biphenylyl)-1-{[1-(cyclopropylcarbonyl)-3-pyrrolidinyl]methyl}-5-methyl-1H-imidazole;
1-{[1-(cyclopropylcarbonyl)-3-pyrrolidinyl]methyl}-5-methyl-2-(3′-methyl-4-biphenylyl)-1H-imidazole;
2-(3′-chloro-4-biphenylyl)-1-{[1-(cyclopropylcarbonyl)-3-pyrrolidinyl]methyl}-5-methyl-1H-imidazole;
1-{[1-(cyclopropylcarbonyl)-3-pyrrolidinyl]methyl}-2-(4′-fluoro-4-biphenylyl)-5-methyl-1H-imidazole;
1-{[1-(cyclopropylcarbonyl)-3-pyrrolidinyl]methyl}-2-(2′,4′-dimethyl-4-biphenylyl)-5-methyl-1H-imidazole;
1-{[1-(cyclopropylcarbonyl)-3-pyrrolidinyl]methyl}-2-(2′,4′-dichloro-4-biphenylyl)-5-methyl-1H-imidazole;
1-{[1-(cyclopropylcarbonyl)-3-pyrrolidinyl]methyl}-2-(4′-fluoro-3′-methyl-4-biphenylyl)-5-methyl-1H-imidazole;
2-[2′-chloro-4′-(methyloxy)-4-biphenylyl]-1-{[1-(cyclopropylcarbonyl)-3-pyrrolidinyl]methyl}-5-methyl-1H-imidazole;
2-(3′-chloro-4′-fluoro-4-biphenylyl)-1-{[1-(cyclopropylcarbonyl)-3-pyrrolidinyl]methyl}-5-methyl-1H-imidazole;
3-[4-(1-benzofuran-5-yl)phenyl]-4-{[(3S)-1-(cyclopropylcarbonyl)-3-pyrrolidinyl]methyl}-4H-1,2,4-triazole;
5-[4-(4-{[(35)-1-(cyclopropylcarbonyl)-3-pyrrolidinyl]methyl}-4H-1,2,4-triazol-3-yl)phenyl]-1H-indole;
5-[4-(4-{[(35)-1-(cyclopropylcarbonyl)-3-pyrrolidinyl]methyl}-4H-1,2,4-triazol-3-yl)phenyl]-1H-indazole;
4-{[(3S)-1-(cyclopropylcarbonyl)-3-pyrrolidinyl]methyl}-3-[4′-(methyloxy)-4-biphenylyl]-4H-1,2,4-triazole;
4′-(4-{[(3S)-1-(cyclopropylcarbonyl)-3-pyrrolidinyl]methyl}-4H-1,2,4-triazol-3-yl)-4-biphenylcarbonitrile;
4-{[(3S)-1-(cyclopropylcarbonyl)-3-pyrrolidinyl]methyl}-3-(2′,4′-difluoro-4-biphenylyl)-4H-1,2,4-triazole;
4′-(4-{[(3S)-1-(cyclopropylcarbonyl)-3-pyrrolidinyl]methyl}-4H-1,2,4-triazol-3-yl)-2-biphenylcarbonitrile;
6-[4-(4-{[(3S)-1-(cyclopropylcarbonyl)-3-pyrrolidinyl]methyl}-4H-1,2,4-triazol-3-yl)phenyl]-1H-pyrrolo[3,2-b]pyridine;
4-[4-(4-{[(3S)-1-(cyclopropylcarbonyl)-3-pyrrolidinyl]methyl}-4H-1,2,4-triazol-3-yl)phenyl]-1H-indole;
4-[4-(4-{[(3S)-1-(cyclopropylcarbonyl)-3-pyrrolidinyl]methyl}-4H-1,2,4-triazol-3-yl)phenyl]-1H-indazole;
7-[4-(4-{[(3S)-1-(cyclopropylcarbonyl)-3-pyrrolidinyl]methyl}-4H-1,2,4-triazol-3-yl)phenyl]imidazo[1,2-a]pyridine;
N′-[4′-(4-{[(3S)-1-(cyclopropylcarbonyl)-3-pyrrolidinyl]methyl}-4H-1,2,4-triazol-3-yl)-3-biphenylyl]-N,N-dimethylsulfamide;
6-[4-(4-{[(3S)-1-(cyclopropylcarbonyl)-3-pyrrolidinyl]methyl}-4H-1,2,4-triazol-3-yl)-3-fluorophenyl]-1H-indole;
3-[4-(1-benzofuran-5-yl)-2-fluorophenyl]-4-{[(3S)-1-(cyclopropylcarbonyl)-3-pyrrolidinyl]methyl}-4H-1,2,4-triazole;
5-[4-(4-{[(3S)-1-(cyclopropylcarbonyl)-3-pyrrolidinyl]methyl}-4H-1,2,4-triazol-3-yl)phenyl]-2,3-dihydro-1H-indole;
5-[4-(4-{[(3S)-1-(cyclopropylcarbonyl)-3-pyrrolidinyl]methyl}-4H-1,2,4-triazol-3-yl)phenyl]-1-methyl-1H-pyrrolo[2,3-b]pyridine;
4-{[(3S)-1-(cyclopropylcarbonyl)-3-pyrrolidinyl]methyl}-3-[4-(2,3-dihydro-1-benzofuran-5-yl)phenyl]-4H-1,2,4-triazole;
5-[4-(4-{[(3S)-1-(cyclopropylcarbonyl)-3-pyrrolidinyl]methyl}-4H-1,2,4-triazol-3-yl)phenyl]-1-methyl-1H-indole;
5-[4′-(4-{[(3S)-1-(cyclopropylcarbonyl)-3-pyrrolidinyl]methyl}-4H-1,2,4-triazol-3-yl)-3-biphenylyl]-1H-tetrazole;
6-[4-(4-{[(3S)-1-(cyclopropylcarbonyl)-3-pyrrolidinyl]methyl}-4H-1,2,4-triazol-3-yl)phenyl]-1H-indazole;
5-[4-(4-{[1-(cyclopropylcarbonyl)-3-azetidinyl]methyl}-4H-1,2,4-triazol-3-yl)phenyl]-1H-indazole;
6-[4-(4-{[1-(cyclopropylcarbonyl)-3-azetidinyl]methyl}-4H-1,2,4-triazol-3-yl)phenyl]-1H-indole;
6-[4-(4-{[1-(cyclopropylcarbonyl)-3-azetidinyl]methyl}-4H-1,2,4-triazol-3-yl)phenyl]-1,3-benzothiazole;
4-{[(3S)-1-(cyclopropylcarbonyl)-3-pyrrolidinyl]methyl}-3-(3′-methyl-4-biphenylyl)-4H-1,2,4-triazole;
4-{[(3S)-1-(cyclopropylcarbonyl)-3-pyrrolidinyl]methyl}-3-(4′-methyl-4-biphenylyl)-4H-1,2,4-triazole;
3-(3′-chloro-4-biphenylyl)-4-{[(3S)-1-(cyclopropylcarbonyl)-3-pyrrolidinyl]methyl}-4H-1,2,4-triazole;
3-(4′-chloro-4-biphenylyl)-4-{[(3S)-1-(cyclopropylcarbonyl)-3-pyrrolidinyl]methyl}-4H-1,2,4-triazole;
6-[4-(4-{[1-(cyclopropylcarbonyl)-3-azetidinyl]methyl}-4H-1,2,4-triazol-3-yl)-3-fluorophenyl]-1H-indole;
4-{[(3S)-1-(cyclopropylcarbonyl)-3-pyrrolidinyl]methyl}-3-[3-fluoro-4′-(1H-pyrazol-1-yl)-4-biphenylyl]-4H-1,2,4-triazole;
4-{[(3S)-1-(cyclopropylcarbonyl)-3-pyrrolidinyl]methyl}-3-[3-fluoro-3′-(1H-pyrazol-5-yl)-4-biphenylyl]-4H-1,2,4-triazole;
4′-(4-{[(3S)-1-(cyclopropylcarbonyl)-3-pyrrolidinyl]methyl}-4H-1,2,4-triazol-3-yl)-3-biphenylol;
4′-(4-{[(3S)-1-(cyclopropylcarbonyl)-3-pyrrolidinyl]methyl}-4H-1,2,4-triazol-3-yl)-4-biphenylol;
4-{[(3S)-1-(cyclopropylcarbonyl)-3-pyrrolidinyl]methyl}-3-(2′,3,4′-trifluoro-4-biphenylyl)-4H-1,2,4-triazole;
4-{[(3S)-1-(cyclopropylcarbonyl)-3-pyrrolidinyl]methyl}-3-(3′,3,4′-trifluoro-4-biphenylyl)-4H-1,2,4-triazole;
4-{[(3S)-1-(cyclopropylcarbonyl)-3-pyrrolidinyl]methyl}-3-[3,4′-difluoro-3′-(methyl)-4-biphenylyl]-4H-1,2,4-triazole;
4-{[(3S)-1-(cyclopropylcarbonyl)-3-pyrrolidinyl]methyl}-3-[3,4′-difluoro-3′-(methyloxy)-4-biphenylyl]-4H-1,2,4-triazole;
4-{[(3S)-1-(cyclopropylcarbonyl)-3-pyrrolidinyl]methyl}-3-[2′,3-difluoro-4′-(methyloxy)-4-biphenylyl]-4H-1,2,4-triazole;
4-{[(3S)-1-(cyclopropylcarbonyl)-3-pyrrolidinyl]methyl}-3-[3′,3-difluoro-4′-(methyloxy)-4-biphenylyl]-4H-1,2,4-triazole;
6-[4-(4-{[1-(cyclopropylcarbonyl)-3-azetidinyl]methyl}-4H-1,2,4-triazol-3-yl)-3-fluorophenyl]quinoline;
4-{[(3S)-1-(cyclopropylcarbonyl)-3-pyrrolidinyl]methyl}-3-(2′,3-difluoro-4′-methyl-4-biphenylyl)-4H-1,2,4-triazole;
4-{[(3S)-1-(cyclopropylcarbonyl)-3-pyrrolidinyl]methyl}-3-[3-fluoro-4′-methyl-3′-(methyloxy)-4-biphenylyl]-4H-1,2,4-triazole;
4-{[(3S)-1-(cyclopropylcarbonyl)-3-pyrrolidinyl]methyl}-3-[3-fluoro-3′-methyl-4′-(methyloxy)-4-biphenylyl]-4H-1,2,4-triazole;
3-[3′-chloro-3-fluoro-4′-(methyloxy)-4-biphenylyl]-4-{[(3S)-1-(cyclopropylcarbonyl)-3-pyrrolidinyl]methyl}-4H-1,2,4-triazole;
7-[4-(4-{[1-(cyclopropylcarbonyl)-3-azetidinyl]methyl}-4H-1,2,4-triazol-3-yl)-3-fluorophenyl]quinoline;
4′-(4-{[(3S)-1-(cyclopropylcarbonyl)-3-pyrrolidinyl]methyl}-4H-1,2,4-triazol-3-yl)-3′,4-difluoro-3-biphenylol;
4′-(4-{[(3S)-1-(cyclopropylcarbonyl)-3-pyrrolidinyl]methyl}-4H-1,2,4-triazol-3-yl)-3′-fluoro-4-(methyloxy)-3-biphenylol;
4′-(4-{[(3S)-1-(cyclopropylcarbonyl)-3-pyrrolidinyl]methyl}-4H-1,2,4-triazol-3-yl)-3′-fluoro-4-biphenylcarbonitrile;
4-{[(3S)-1-(cyclopropylcarbonyl)-3-pyrrolidinyl]methyl}-3-(3,4′-difluoro-4-biphenylyl)-4H-1,2,4-triazole;
4′-(4-{[(3S)-1-(cyclopropylcarbonyl)-3-pyrrolidinyl]methyl}-4H-1,2,4-triazol-3-yl)-3′-fluoro-N,N-dimethyl-4-biphenylamine;
7-[4-(4-{[(3S)-1-(cyclopropylcarbonyl)-3-pyrrolidinyl]methyl}-4H-1,2,4-triazol-3-yl)-3-fluorophenyl]quinoline;
3-[4-(4-{[(3S)-1-(cyclopropylcarbonyl)-3-pyrrolidinyl]methyl}-4H-1,2,4-triazol-3-yl)-3-fluorophenyl]quinoline; and
4′-(4-{[(3S)-1-(cyclopropylcarbonyl)-3-pyrrolidinyl]methyl}-4H-1,2,4-triazol-3-yl)-3′-fluoro-4-methyl-3-biphenylol;

and pharmaceutically acceptable salts thereof.

This invention also relates to compounds exemplified in the Experimental section.

Typically, but not absolutely, the salts of the present invention are pharmaceutically acceptable salts. Salts encompassed within the term “pharmaceutically acceptable salts” refer to non-toxic salts of the compounds of this invention. Salts of the compounds of the present invention may comprise acid addition salts. In general, the salts are formed from pharmaceutically acceptable inorganic and organic acids. More specific examples of suitable acid salts include maleic, hydrochloric, hydrobromic, sulphuric, phosphoric, nitric, perchloric, fumic, acetic, propionic, succinic, glycolic, formic, lactic, aleic, tartaric, citric, palmoic, malonic, hydroxymaleic, phenylacetic, glutamic, benzoic, salicylic, fumaric, toluenesulfonic, methansulfonic (mesylate), naphthalene-2-sulfonic, benzenesulfonic, hydroxynaphthoic, hydroiodic, malic, teroic, tannic, and the like.

Other representative salts include acetate, benzenesulfonate, benzoate, bicarbonate, bisulfate, bitartrate, borate, calcium edetate, camsylate, carbonate, clavulanate, citrate, dihydrochloride, edisylate, estolate, esylate, fumarate, gluceptate, gluconate, glutamate, glycollylarsanilate, hexylresorcinate, hydrobromide, hydrochloride, hydroxynaphthoate, iodide, isethionate, lactate, lactobionate, laurate, malate, maleate, mandelate, mesylate, methylsulfate, monopotassium maleate, mucate, napsylate, nitrate, oxalate, pamoate (embonate), palmitate, pantothenate, phosphate/diphosphate, polygalacturonate, salicylate, stearate, subacetate, succinate, sulfate, tannate, tartrate, teoclate, tosylate, triethiodide, and valerate salts.

Other salts, which are not pharmaceutically acceptable, may be useful in the preparation of compounds of this invention and these should be considered to form a further aspect of the invention. These salts, such as oxalic or trifluoroacetate, while not in themselves pharmaceutically acceptable, may be useful in the preparation of salts useful as intermediates in obtaining the compounds of the invention and their pharmaceutically acceptable salts.

The compound of Formula (I) or a salt thereof may exist in stereoisomeric forms (e.g., it contains one or more asymmetric carbon atoms). The individual stereoisomers (enantiomers and diastereomers) and mixtures of these are included within the scope of the present invention. The invention also covers the individual isomers of the compound or salt represented by Formula (I) as mixtures with isomers thereof in which one or more chiral centers are inverted. Likewise, it is understood that a compound or salt of Formula (I) may exist in tautomeric forms other than that shown in the formula and these are also included within the scope of the present invention. It is to be understood that the present invention includes all combinations and subsets of the particular groups defined hereinabove. The scope of the present invention includes mixtures of stereoisomers as well as purified enantiomers or enantiomerically/diastereomerically enriched mixtures. Also included within the scope of the invention are individual isomers of the compound represented by Formula (I), as well as any wholly or partially equilibrated mixtures thereof. The present invention also includes the individual isomers of the compound or salt represented by the Formula (I) as well as mixtures with isomers thereof in which one or more chiral centers are inverted. It is to be understood that the present invention includes all combinations and subsets of the particular groups defined hereinabove.

The invention also includes various deuterated forms of the compounds of Formula (I). Each available hydrogen atom attached to a carbon atom may be independently replaced with a deuterium atom. A person of ordinary skill in the art will know how to synthesize deuterated forms of the compounds of Formula (I). Commercially available deuterated starting materials may be employed in the preparation of deuterated forms of the compounds of Formula (I), or they may be synthesized using conventional techniques employing deuterated reagents (e.g. lithium aluminum deuteride).

### DEFINITIONS

Terms are used within their accepted meanings. The following definitions are meant to clarify, but not limit, the terms defined.

As used herein, the term “alkyl” refers to a straight or branched chain hydrocarbon radical, preferably having from one to twelve carbon atoms, which may be unsubstituted or substituted, saturated or unsaturated with multiple degrees of substitution included within the present invention. When optionally substituted, the alkyl group is unsubstituted or substituted with suitable substituents selected from the group consisting of halogen, amino, substituted amino, cyano, hydroxyl, alkoxy, alkylthio, alkylsulfonyl, aminosulfonyl, carboxylic acid, carboxylic ester, carboxamide, aminocarbonyl, and heterocyclyl. Examples of “alkyl” as used herein include, but are not limited to, methyl, ethyl, propyl, isopropyl, isobutyl, n-butyl, t-butyl, isopentyl, n-pentyl, and the like, as well as substituted versions thereof.

As used herein, the term “cycloalkyl” refers to an unsubstituted or substituted mono- or polycyclic non-aromatic saturated ring. Exemplary “cycloalkyl” groups include, but are not limited to, cyclopropyl, cyclobutyl, cyclopentyl, cyclohexyl, cycloheptyl, and the like, as well as unsubstituted and substituted versions thereof.

As used herein, the term “alkoxy” refers to the group —OR^{a}, where R^{a} is (C₁-C₄)alkyl or (C₃-C₇)cycloalkyl as defined above.

As used herein, the term “heterocyclyl” refers to an unsubstituted or substituted mono- or polycyclic ring system containing one or more heteroatoms. Preferred heteroatoms include nitrogen, oxygen, and sulfur, including N-oxides, sulfur oxides, and dioxides. The term “9- or 10-membered heterocyclyl” represents a fully unsaturated or partially unsaturated, bicyclic group, containing 9 or 10 ring atoms, including 1 to 5 heteroatoms independently selected from nitrogen, oxygen and sulfur, which group may be unsubstituted or substituted by one or more of the substituents defined herein. Selected 9- or 10-membered heterocycyl groups contain one nitrogen, oxygen or sulfur ring heteroatom, and optionally contain 1, 2, 3, or 4 additional nitrogen ring atoms and/or 1 additional oxygen or sulfur atom. Examples of 9- or 10-membered heterocyclyl groups include, but are not limited to benzofuranyl, isobenzofuryl, 2,3-dihydrobenzofuryl, 1,3-benzodioxolyl, dihydrobenzodioxinyl, benzothienyl, indolizinyl, indolyl, isoindolyl, indolinyl, isoindolinyl, benzimidazolyl, dihydrobenzimidazolyl, benzoxazolyl, dihydrobenzoxazolyl, benzthiazolyl, benzoisothiazolyl, dihydrobenzoisothiazolyl, indazolyl, pyrrolopyridinyl, pyrrolopyrimidinyl, imidazopyridinyl, imidazopyrimidinyl, pyrazolopyridinyl, pyrazolopyrimidinyl, benzoxadiazolyl, benzthiadiazolyl, benzotriazolyl, triazolopyridinyl, purinyl, quinolinyl, tetrahydroquinolinyl, isoquinolinyl, tetrahydroisoquinolinyl, quinoxalinyl, cinnolinyl, phthalazinyl, quinazolinyl, 1,5-naphthyridinyl, 1,6-naphthyridinyl, 1,7-naphthyridinyl, 1,8-naphthyridinyl, and pteridinyl.

As used herein, the term “heteroaryl”, unless otherwise defined, refers to an aromatic ring system containing carbon atom(s) and at least one heteroatom. Heteroaryl may be monocyclic or polycyclic, substituted or unsubstituted. A monocyclic heteroaryl group may have 1 to 4 heteroatoms in the ring, while a polycyclic heteroaryl may contain 1 to 10 heteroatoms. Bicyclic heteroaryl rings may contain from 8 to 10 member atoms. The 5-membered heteroaryl groups present in the compounds of this invention contain one nitrogen, oxygen, or sulfur ring heteroatom, and optionally contain 1, 2, or 3 additional nitrogen ring atoms. The 6-membered heteroaryl groups present in the compounds of this invention contain 1, 2, 3, or 4 nitrogen ring heteroatoms. Examples of 5- or 6-membered heteroaryl groups include, but are not limited to furanyl, thienyl, pyrrolyl, imidazolyl, pyrazolyl, triazolyl, tetrazolyl, thiazolyl, oxazolyl, isoxazolyl, oxadiazolyl, thiadiazolyl, isothiazolyl, pyridinyl, pyridazinyl, pyrazinyl, pyrimidinyl, and triazinyl.

As used herein, the term “cyano” refers to the group —CN.

As used herein, the term “optionally” means that the subsequently described event(s) may or may not occur, and includes both event(s) that occur and event(s) that do not occur.

As used herein, unless otherwise defined, the phrase “optionally substituted” or variations thereof denote an optional substitution, including multiple degrees of substitution, with one or more substitutent group. The phrase should not be interpreted as duplicative of the substitutions herein described and depicted. Exemplary optional substituent groups include acyl, alkyl, alkylsulfonyl, alkoxy, alkoxycarbonyl, cyano, halogen, haloalkyl, hydroxyl, oxo, amide, sulfamide, urea, amino, substituted amino, acylamino, phenylcarbonyl, dialkylaminosulfonamide, morpholino, sulfonamide, thiourea, nitro, pyrrolidinyl, pyrazolyl, pyrrolyl, phenyl, and tetrazolyl, wherein pyrrolidinyl, pyrazolyl and tetrazolyl can be further substituted with one to three (C₁-C₃)alkyl.

The invention further provides a pharmaceutical composition (also referred to as pharmaceutical formulation) comprising a compound of Formula (I) or pharmaceutically acceptable salt, thereof and one or more excipients (also referred to as carriers and/or diluents in the pharmaceutical arts). The excipients are acceptable in the sense of being compatible with the other ingredients of the formulation and not deleterious to the recipient thereof (i.e., the patient).

In accordance with another aspect of the invention there is provided a process for the preparation of a pharmaceutical composition comprising mixing (or admixing) a compound of Formula (I) or salt thereof with at least one excipient.

### Pharmaceutical Compositions

Pharmaceutical compositions may be in unit dose form containing a predetermined amount of active ingredient per unit dose. Such a unit may contain a therapeutically effective dose of the compound of Formula (I) or salt thereof or a fraction of a therapeutically effective dose such that multiple unit dosage forms might be administered at a given time to achieve the desired therapeutically effective dose. Preferred unit dosage formulations are those containing a daily dose or sub-dose, as herein above recited, or an appropriate fraction thereof, of an active ingredient. Furthermore, such pharmaceutical compositions may be prepared by any of the methods well-known in the pharmacy art.

Pharmaceutical compositions may be adapted for administration by any appropriate route, for example, by oral (including buccal or sublingual), rectal, nasal, topical (including buccal, sublingual, or transdermal), vaginal, or parenteral (including subcutaneous, intramuscular, intravenous, or intradermal) routes. Such compositions may be prepared by any method known in the art of pharmacy, for example, by bringing into association the active ingredient with the excipient(s).

When adapted for oral administration, pharmaceutical compositions may be in discrete units such as tablets or capsules; powders or granules; solutions or suspensions in aqueous or non-aqueous liquids; edible foams or whips; oil-in-water liquid emulsions or water-in-oil liquid emulsions. The compound or salt thereof of the invention or the pharmaceutical composition of the invention may also be incorporated into a candy, a wafer, and/or tongue tape formulation for administration as a “quick-dissolve” medicine.

For instance, for oral administration in the form of a tablet or capsule, the active drug component can be combined with an oral, non-toxic pharmaceutically acceptable inert carrier such as ethanol, glycerol, water, and the like. Powders or granules are prepared by comminuting the compound to a suitable fine size and mixing with a similarly comminuted pharmaceutical carrier such as an edible carbohydrate, as, for example, starch or mannitol. Flavoring, preservative, dispersing, and coloring agents can also be present.

Capsules are made by preparing a powder mixture, as described above, and filling formed gelatin or non-gelatinous sheaths. Glidants and lubricants such as colloidal silica, talc, magnesium stearate, calcium stearate, solid polyethylene glycol can be added to the powder mixture before the filling operation. A disintegrating or solubilizing agent such as agar-agar, calcium carbonate, or sodium carbonate can also be added to improve the availability of the medicine when the capsule is ingested.

Moreover, when desired or necessary, suitable binders, lubricants, disintegrating agents, and coloring agents can also be incorporated into the mixture. Suitable binders include starch, gelatin, natural sugars, such as glucose or beta-lactose, corn sweeteners, natural and synthetic gums such as acacia, tragacanth, sodium alginate, carboxymethylcellulose, polyethylene glycol, waxes, and the like. Lubricants used in these dosage forms include sodium oleate, sodium stearate, magnesium stearate, sodium benzoate, sodium acetate, sodium chloride, and the like. Disintegrators include, without limitation, starch, methylcellulose, agar, bentonite, xanthan gum, and the like.

Tablets are formulated, for example, by preparing a powder mixture, granulating or slugging, adding a lubricant and disintegrant, and pressing into tablets. A powder mixture is prepared by mixing the compound, suitably comminuted, with a diluent or base as described above, and optionally, with a binder such as carboxymethylcellulose, and aliginate, gelatin, or polyvinyl pyrrolidone, a solution retardant such as paraffin, a resorption accelerator such as a quaternary salt, and/or an absorption agent such as bentonite, kaolin, or dicalcium phosphate. The powder mixture can be granulated by wetting a binder such as syrup, starch paste, acadia mucilage, or solutions of cellulosic or polymeric materials and forcing through a screen. As an alternative to granulating, the powder mixture can be run through the tablet machine and the result is imperfectly formed slugs broken into granules. The granules can be lubricated to prevent sticking to the tablet forming dies by means of the addition of stearic acid, a stearate salt, talc, or mineral oil. The lubricated mixture is then compressed into tablets. The compound or salt of the present invention can also be combined with a free-flowing inert carrier and compressed into tablets directly without going through the granulating or slugging steps. A clear opaque protective coating consisting of a sealing coat of shellac, a coating of sugar, or polymeric material, and a polish coating of wax can be provided. Dyestuffs can be added to these coatings to distinguish different dosages.

Oral fluids such as solutions, syrups, and elixirs can be prepared in dosage unit form so that a given quantity contains a predetermined amount of active ingredient. Syrups can be prepared by dissolving the compound or salt thereof of the invention in a suitably flavoured aqueous solution, while elixirs are prepared through the use of a non-toxic alcoholic vehicle. Suspensions can be formulated by dispersing the compound or salt of the invention in a non-toxic vehicle. Solubilizers and emulsifiers, such as ethoxylated isostearyl alcohols and polyoxyethylene sorbitol ethers, preservatives, flavor additives such as peppermint oil, natural sweeteners, saccharin, or other artificial sweeteners, and the like, can also be added.

Where appropriate, dosage unit formulations for oral administration can be microencapsulated. The formulation can also be prepared to prolong or sustain the release as, for example, by coating or embedding particulate material in polymers, wax, or the like.

In the present invention, tablets and capsules are preferred for delivery of the pharmaceutical composition.

As used herein, the term “treatment” includes prophylaxis and refers to alleviating the specified condition, eliminating or reducing one or more symptoms of the condition, slowing or eliminating the progression of the condition, and preventing or delaying the reoccurrence of the condition in a previously afflicted or diagnosed patient or subject. Prophylaxis (or prevention or delay of disease onset) is typically accomplished by administering a drug in the same or similar manner as one would to a patient with the developed disease or condition.

The present invention provides a method of treatment in a mammal, especially a human, suffering from disease conditions targeted by the present compounds. Such treatment comprises the step of administering a therapeutically effective amount of a compound of Formula (I) or salt thereof to said mammal, particularly a human. Treatment can also comprise the step of administering a therapeutically effective amount of a pharmaceutical composition containing a compound of Formula (I) or salt thereof to said mammal, particularly a human.

As used herein, the term “effective amount” means that amount of a drug or pharmaceutical agent that will elicit the biological or medical response of a tissue, system, animal, or human that is being sought, for instance, by a researcher or clinician.

The term “therapeutically effective amount” means any amount which, as compared to a corresponding subject who has not received such amount, results in improved treatment, healing, prevention, or amelioration of a disease, disorder, or side effect, or a decrease in the rate of advancement of a disease or disorder. The term also includes within its scope amounts effective to enhance normal physiological function. For use in therapy, therapeutically effective amounts of a compound of Formula (I), as well as salts thereof, may be administered as the raw chemical. Additionally, the active ingredient may be presented as a pharmaceutical composition.

While it is possible that, for use in therapy, a therapeutically effective amount of a compound of Formula (I) or salt thereof may be administered as the raw chemical, it is typically presented as the active ingredient of a pharmaceutical composition or formulation.

The precise therapeutically effective amount of a compound or salt thereof of the invention will depend on a number of factors, including, but not limited to, the age and weight of the subject (patient) being treated, the precise disorder requiring treatment and its severity, the nature of the pharmaceutical formulation/composition, and route of administration, and will ultimately be at the discretion of the attending physician or veterinarian. Typically, a compound of Formula (I) or salt thereof will be given for the treatment in the range of about 0.1 to 100 mg/kg body weight of recipient (patient, mammal) per day and more usually in the range of 0.1 to 10 mg/kg body weight per day. Acceptable daily dosages may be from about 1 to about 1000 mg/day, and preferably from about 1 to about 100 mg/day. This amount may be given in a single dose per day or in a number (such as two, three, four, five, or more) of sub-doses per day such that the total daily dose is the same. An effective amount of a salt thereof may be determined as a proportion of the effective amount of the compound of Formula (I) per se. Similar dosages should be appropriate for treatment (including prophylaxis) of the other conditions referred herein for treatment. In general, determination of appropriate dosing can be readily arrived at by one skilled in medicine or the pharmacy art.

Combinations

When a compound of Formula (I) is administered for the treatment of cancer, the term “co-administering” and derivatives thereof as used herein is meant either simultaneous administration or any manner of separate sequential administration of a FAS inhibiting compound, as described herein, and a further active ingredient or ingredients, known to be useful in the treatment of cancer, including chemotherapy and radiation treatment. The term further active ingredient or ingredients, as used herein, includes any compound or therapeutic agent known to or that demonstrates advantageous properties when administered to a patient in need of treatment for cancer. Preferably, if the administration is not simultaneous, the compounds are administered in a close time proximity to each other. Furthermore, it does not matter if the compounds are administered in the same dosage form, e.g. one compound may be administered topically and another compound may be administered orally.

Typically, any anti-neoplastic agent that has activity versus a susceptible tumor being treated may be co-administered in the treatment of cancer in the present invention. Examples of such agents can be found in Cancer Principles and Practice f Oncology by V. T. Devita and S. Hellman (editors), 6^{th} edition (Feb. 15, 2001), Lippincott Williams & Wilkins Publishers. A person of ordinary skill in the art would be able to discern which combinations of agents would be useful based on the particular characteristics of the drugs and the cancer involved. Typical anti-neoplastic agents useful in the present invention include, but are not limited to, anti-microtubule agents such as diterpenoids and vinca alkaloids; platinum coordination complexes; alkylating agents such as nitrogen mustards, oxazaphosphorines, alkylsulfonates, nitrosoureas, and triazenes; antibiotic agents such as anthracycline, actinomycins and bleomycins; topoisomerase II inhibitors such as epipodophyllotoxins; antimetabolites such as purine and pyrimidine analogues and anti-folate compounds; topoisomerase I inhibitors such as camptothecins; hormones and hormonal analogues; signal transduction pathway inhibitors; non-receptor tyrosine kinase angiogenesis inhibitors; immunotherapeutic agents; proapoptotic agents; and cell cycle signaling inhibitors.

Examples of a further active ingredient or ingredients for use in combination or co-administered with the present FAS inhibiting compounds are chemotherapeutic agents.

Anti-microtubule or anti-mitotic agents are phase specific agents active against the microtubules of tumor cells during M or the mitosis phase of the cell cycle. Examples of anti-microtubule agents include, but are not limited to, diterpenoids and vinca alkaloids.

Diterpenoids, which are derived from natural sources, are phase specific anti-cancer agents that operate at the G₂/M phases of the cell cycle. It is believed that the diterpenoids stabilize the β-tubulin subunit of the microtubules, by binding with this protein. Disassembly of the protein appears then to be inhibited with mitosis being arrested and cell death following. Examples of diterpenoids include, but are not limited to, paclitaxel and its analog docetaxel.

Paclitaxel, 5β,20-epoxy-1,2α,4,7β,10β,13α-hexa-hydroxytax-11-en-9-one 4,10-diacetate 2-benzoate 13-ester with (2R,3S)—N-benzoyl-3-phenylisoserine; is a natural diterpene product isolated from the Pacific yew tree Taxus brevifolia and is commercially available as an injectable solution TAXOL®. It is a member of the taxane family of terpenes. It was first isolated in 1971 by Wani et al. J. Am. Chem., Soc., 93:2325. 1971), who characterized its structure by chemical and X-ray crystallographic methods. One mechanism for its activity relates to paclitaxel's capacity to bind tubulin, thereby inhibiting cancer cell growth. Schiff et al., Proc. Natl, Acad, Sci. USA, 77:1561-1565 (1980); Schiff et al., Nature, 277:665-667 (1979); Kumar, J. Biol, Chem, 256: 10435-10441 (1981). For a review of synthesis and anticancer activity of some paclitaxel derivatives see: D. G. I. Kingston et al., Studies in Organic Chemistry vol. 26, entitled “New trends in Natural Products Chemistry 1986”, Attaur-Rahman, P. W. Le Quesne, Eds. (Elsevier, Amsterdam, 1986) pp 219-235.

Paclitaxel has been approved for clinical use in the treatment of refractory ovarian cancer in the United States (Markman et al., Yale Journal of Biology and Medicine, 64:583, 1991; McGuire et al., Ann. Intem, Med., 111:273, 1989) and for the treatment of breast cancer (Holmes et al., J. Nat. Cancer Inst., 83:1797, 1991.) It is a potential candidate for treatment of neoplasms in the skin (Einzig et. al., Proc. Am. Soc. Clin. Oncol., 20:46) and head and neck carcinomas (Forastire et. al., Sem. Oncol., 20:56, 1990). The compound also shows potential for the treatment of polycystic kidney disease (Woo et. al., Nature, 368:750. 1994), lung cancer and malaria. Treatment of patients with paclitaxel results in bone marrow suppression (multiple cell lineages, Ignoff, R. J. et. al, Cancer Chemotherapy Pocket Guide, 1998) related to the duration of dosing above a threshold concentration (50 nM) (Kearns, C. M. et. al., Seminars in Oncology, 3(6) p. 16-23, 1995).

Docetaxel, (2R,3S)—N-carboxy-3-phenylisoserine,N-tert-butyl ester, 13-ester with 5β-20-epoxy-1,2α,4,7β,10β,13α-hexahydroxytax-11-en-9-one 4-acetate 2-benzoate, trihydrate; is commercially available as an injectable solution as TAXOTERE®. Docetaxel is indicated for the treatment of breast cancer. Docetaxel is a semisynthetic derivative of paclitaxel q.v., prepared using a natural precursor, 10-deacetyl-baccatin III, extracted from the needle of the European Yew tree. The dose limiting toxicity of docetaxel is neutropenia.

Vinca alkaloids are phase specific anti-neoplastic agents derived from the periwinkle plant. Vinca alkaloids act at the M phase (mitosis) of the cell cycle by binding specifically to tubulin. Consequently, the bound tubulin molecule is unable to polymerize into microtubules. Mitosis is believed to be arrested in metaphase with cell death following. Examples of vinca alkaloids include, but are not limited to, vinblastine, vincristine, and vinorelbine.

Vinblastine, vincaleukoblastine sulfate, is commercially available as VELBAN® as an injectable solution. Although, it has possible indication as a second line therapy of various solid tumors, it is primarily indicated in the treatment of testicular cancer and various lymphomas including Hodgkin's Disease; and lymphocytic and histiocytic lymphomas. Myelosuppression is the dose limiting side effect of vinblastine.

Vincristine, vincaleukoblastine, 22-oxo-, sulfate, is commercially available as ONCOVIN® as an injectable solution. Vincristine is indicated for the treatment of acute leukemias and has also found use in treatment regimens for Hodgkin's and non-Hodgkin's malignant lymphomas. Alopecia and neurologic effects are the most common side effect of vincristine and to a lesser extent myelosupression and gastrointestinal mucositis effects occur.

Vinorelbine, 3′,4′-didehydro-4′-deoxy-C′-norvincaleukoblastine [R—(R*,R*)-2,3-dihydroxybutanedioate (1:2)(salt)], commercially available as an injectable solution of vinorelbine tartrate (NAVELBINE®), is a semisynthetic vinca alkaloid. Vinorelbine is indicated as a single agent or in combination with other chemotherapeutic agents, such as cisplatin, in the treatment of various solid tumors, particularly non-small cell lung, advanced breast, and hormone refractory prostate cancers. Myelosuppression is the most common dose limiting side effect of vinorelbine.

Platinum coordination complexes are non-phase specific anti-cancer agents, which are interactive with DNA. The platinum complexes enter tumor cells, undergo, aquation and form intra- and interstrand crosslinks with DNA causing adverse biological effects to the tumor. Examples of platinum coordination complexes include, but are not limited to, cisplatin and carboplatin.

Cisplatin, cis-diamminedichloroplatinum, is commercially available as PLATINOL® as an injectable solution. Cisplatin is primarily indicated in the treatment of metastatic testicular and ovarian cancer and advanced bladder cancer. The primary dose limiting side effects of cisplatin are nephrotoxicity, which may be controlled by hydration and diuresis, and ototoxicity.

Carboplatin, platinum, diammine [1,1-cyclobutane-dicarboxylate(2-)-O,O′], is commercially available as PARAPLATIN® as an injectable solution. Carboplatin is primarily indicated in the first and second line treatment of advanced ovarian carcinoma. Bone marrow suppression is the dose limiting toxicity of carboplatin.

Alkylating agents are non-phase anti-cancer specific agents and strong electrophiles. Typically, alkylating agents form covalent linkages, by alkylation, to DNA through nucleophilic moieties of the DNA molecule such as phosphate, amino, sulfhydryl, hydroxyl, carboxyl, and imidazole groups. Such alkylation disrupts nucleic acid function leading to cell death. Examples of alkylating agents include, but are not limited to, nitrogen mustards such as cyclophosphamide, melphalan, and chlorambucil; alkyl sulfonates such as busulfan; nitrosoureas such as carmustine; and triazenes such as dacarbazine.

Cyclophosphamide, 2-[bis(2-chloroethyl)amino]tetrahydro-2H-1,3,2-oxazaphosphorine 2-oxide monohydrate, is commercially available as an injectable solution or tablets as CYTOXAN®. Cyclophosphamide is indicated as a single agent or in combination with other chemotherapeutic agents, in the treatment of malignant lymphomas, multiple myeloma, and leukemias. Alopecia, nausea, vomiting and leukopenia are the most common dose limiting side effects of cyclophosphamide.

Melphalan, 4-[bis(2-chloroethyl)amino]-L-phenylalanine, is commercially available as an injectable solution or tablets as ALKERAN®. Melphalan is indicated for the palliative treatment of multiple myeloma and non-resectable epithelial carcinoma of the ovary. Bone marrow suppression is the most common dose limiting side effect of melphalan.

Chlorambucil, 4-[bis(2-chloroethyl)amino]benzenebutanoic acid, is commercially available as LEUKERAN® tablets. Chlorambucil is indicated for the palliative treatment of chronic lymphatic leukemia, and malignant lymphomas such as lymphosarcoma, giant follicular lymphoma, and Hodgkin's disease. Bone marrow suppression is the most common dose limiting side effect of chlorambucil.

Busulfan, 1,4-butanediol dimethanesulfonate, is commercially available as MYLERAN® TABLETS. Busulfan is indicated for the palliative treatment of chronic myelogenous leukemia. Bone marrow suppression is the most common dose limiting side effects of busulfan.

Carmustine, 1,3-[bis(2-chloroethyl)-1-nitrosourea, is commercially available as single vials of lyophilized material as BiCNU®. Carmustine is indicated for the palliative treatment as a single agent or in combination with other agents for brain tumors, multiple myeloma, Hodgkin's disease, and non-Hodgkin's lymphomas. Delayed myelosuppression is the most common dose limiting side effects of carmustine.

Dacarbazine, 5-(3,3-dimethyl-1-triazeno)-imidazole-4-carboxamide, is commercially available as single vials of material as DTIC-Dome®. Dacarbazine is indicated for the treatment of metastatic malignant melanoma and in combination with other agents for the second line treatment of Hodgkin's Disease. Nausea, vomiting, and anorexia are the most common dose limiting side effects of dacarbazine.

Antibiotic anti-neoplastics are non-phase specific agents, which bind or intercalate with DNA. Typically, such action results in stable DNA complexes or strand breakage, which disrupts ordinary function of the nucleic acids leading to cell death. Examples of antibiotic anti-neoplastic agents include, but are not limited to, actinomycins such as dactinomycin, anthrocyclins such as daunorubicin and doxorubicin; and bleomycins.

Dactinomycin, also know as Actinomycin D, is commercially available in injectable form as COSMEGEN®. Dactinomycin is indicated for the treatment of Wilm's tumor and rhabdomyosarcoma. Nausea, vomiting, and anorexia are the most common dose limiting side effects of dactinomycin.

Daunorubicin, (8S-cis-)-8-acetyl-10-[(3-amino-2,3,6-trideoxy-α-L-lyxo-hexopyranosyl)oxy]-7,8,9,10-tetrahydro-6,8,11-trihydroxy-1-methoxy-5,12 naphthacenedione hydrochloride, is commercially available as a liposomal injectable form as DAUNOXOME® or as an injectable as CERUBIDINE®. Daunorubicin is indicated for remission induction in the treatment of acute nonlymphocytic leukemia and advanced HIV associated Kaposi's sarcoma. Myelosuppression is the most common dose limiting side effect of daunorubicin.

Doxorubicin, (8S,10S)-10-[(3-amino-2,3,6-trideoxy-α-L-lyxo-hexopyranosyl)oxy]-8-glycoloyl, 7,8,9,10-tetrahydro-6,8,11-trihydroxy-1-methoxy-5,12 naphthacenedione hydrochloride, is commercially available as an injectable form as RUBEX® or ADRIAMYCIN RDF®. Doxorubicin is primarily indicated for the treatment of acute lymphoblastic leukemia and acute myeloblastic leukemia, but is also a useful component in the treatment of some solid tumors and lymphomas. Myelosuppression is the most common dose limiting side effect of doxorubicin.

Bleomycin, a mixture of cytotoxic glycopeptide antibiotics isolated from a strain of Streptomyces verticillus, is commercially available as BLENOXANE®. Bleomycin is indicated as a palliative treatment, as a single agent or in combination with other agents, of squamous cell carcinoma, lymphomas, and testicular carcinomas. Pulmonary and cutaneous toxicities are the most common dose limiting side effects of bleomycin.

Topoisomerase II inhibitors include, but are not limited to, epipodophyllotoxins.

Epipodophyllotoxins are phase specific anti-neoplastic agents derived from the mandrake plant. Epipodophyllotoxins typically affect cells in the S and G₂ phases of the cell cycle by forming a ternary complex with topoisomerase II and DNA causing DNA strand breaks. The strand breaks accumulate and cell death follows. Examples of epipodophyllotoxins include, but are not limited to, etoposide and teniposide.

Etoposide, 4′-demethyl-epipodophyllotoxin 9[4,6-0-(R)-ethylidene-β-D-glucopyranoside], is commercially available as an injectable solution or capsules as VePESID® and is commonly known as VP-16. Etoposide is indicated as a single agent or in combination with other chemotherapy agents in the treatment of testicular and non-small cell lung cancers. Myelosuppression is the most common side effect of etoposide. The incidence of leucopenia tends to be more severe than thrombocytopenia.

Teniposide, 4′-demethyl-epipodophyllotoxin 9[4,6-0-(R)-thenylidene-β-D-glucopyranoside], is commercially available as an injectable solution as VUMON® and is commonly known as VM-26. Teniposide is indicated as a single agent or in combination with other chemotherapy agents in the treatment of acute leukemia in children. Myelosuppression is the most common dose limiting side effect of teniposide. Teniposide can induce both leucopenia and thrombocytopenia.

Antimetabolite neoplastic agents are phase specific anti-neoplastic agents that act at S phase (DNA synthesis) of the cell cycle by inhibiting DNA synthesis or by inhibiting purine or pyrimidine base synthesis and thereby limiting DNA synthesis. Consequently, S phase does not proceed and cell death follows. Examples of antimetabolite anti-neoplastic agents include, but are not limited to, fluorouracil, methotrexate, cytarabine, mercaptopurine, thioguanine, and gemcitabine.

5-fluorouracil, 5-fluoro-2,4-(1H,3H) pyrimidinedione, is commercially available as fluorouracil. Administration of 5-fluorouracil leads to inhibition of thymidylate synthesis and is also incorporated into both RNA and DNA. The result typically is cell death. 5-fluorouracil is indicated as a single agent or in combination with other chemotherapy agents in the treatment of carcinomas of the breast, colon, rectum, stomach and pancreas. Myelosuppression and mucositis are dose limiting side effects of 5-fluorouracil. Other fluoropyrimidine analogs include 5-fluoro deoxyuridine (floxuridine) and 5-fluorodeoxyuridine monophosphate.

Cytarabine, 4-amino-1-β-D-arabinofuranosyl-2 (1H)-pyrimidinone, is commercially available as CYTOSAR-U® and is commonly known as Ara-C. It is believed that cytarabine exhibits cell phase specificity at S-phase by inhibiting DNA chain elongation by terminal incorporation of cytarabine into the growing DNA chain. Cytarabine is indicated as a single agent or in combination with other chemotherapy agents in the treatment of acute leukemia. Other cytidine analogs include 5-azacytidine and 2′,2′-difluorodeoxycytidine (gemcitabine). Cytarabine induces leucopenia, thrombocytopenia, and mucositis.

Mercaptopurine, 1,7-dihydro-6H-purine-6-thione monohydrate, is commercially available as PURINETHOL®. Mercaptopurine exhibits cell phase specificity at S-phase by inhibiting DNA synthesis by an as of yet unspecified mechanism. Mercaptopurine is indicated as a single agent or in combination with other chemotherapy agents in the treatment of acute leukemia. Myelosuppression and gastrointestinal mucositis are expected side effects of mercaptopurine at high doses. A useful mercaptopurine analog is azathioprine.

Thioguanine, 2-amino-1,7-dihydro-6H-purine-6-thione, is commercially available as TABLOID®. Thioguanine exhibits cell phase specificity at S-phase by inhibiting DNA synthesis by an as of yet unspecified mechanism. Thioguanine is indicated as a single agent or in combination with other chemotherapy agents in the treatment of acute leukemia. Myelosuppression, including leucopenia, thrombocytopenia, and anemia, is the most common dose limiting side effect of thioguanine administration. However, gastrointestinal side effects occur and can be dose limiting. Other purine analogs include pentostatin, erythrohydroxynonyladenine, fludarabine phosphate, and cladribine.

Gemcitabine, 2′-deoxy-2′,2′-difluorocytidine monohydrochloride (β-isomer), is commercially available as GEMZAR®. Gemcitabine exhibits cell phase specificity at S-phase and by blocking progression of cells through the G1/S boundary. Gemcitabine is indicated in combination with cisplatin in the treatment of locally advanced non-small cell lung cancer and alone in the treatment of locally advanced pancreatic cancer. Myelosuppression, including leucopenia, thrombocytopenia, and anemia, is the most common dose limiting side effect of gemcitabine administration.

Methotrexate, N-[4[[(2,4-diamino-6-pteridinyl)methyl]methylamino]benzoyl]-L-glutamic acid, is commercially available as methotrexate sodium. Methotrexate exhibits cell phase effects specifically at S-phase by inhibiting DNA synthesis, repair and/or replication through the inhibition of dyhydrofolic acid reductase which is required for synthesis of purine nucleotides and thymidylate. Methotrexate is indicated as a single agent or in combination with other chemotherapy agents in the treatment of choriocarcinoma, meningeal leukemia, non-Hodgkin's lymphoma, and carcinomas of the breast, head, neck, ovary and bladder. Myelosuppression (leucopenia, thrombocytopenia, and anemia) and mucositis are expected side effect of methotrexate administration.

Camptothecins, including, camptothecin and camptothecin derivatives are available or under development as Topoisomerase I inhibitors. Camptothecins cytotoxic activity is believed to be related to its Topoisomerase I inhibitory activity. Examples of camptothecins include, but are not limited to irinotecan, topotecan, and the various optical forms of 7-(4-methylpiperazino-methylene)-10,11-ethylenedioxy-20-camptothecin described below.

Irinotecan HCl, (4S)-4,11-diethyl-4-hydroxy-9-[(4-piperidinopiperidino) carbonyloxy]-1H-pyrano[3′,4′,6,7]indolizino[1,2-b]quinoline-3,14(4H,12H)-dione hydrochloride, is commercially available as the injectable solution CAMPTOSAR®.

Irinotecan is a derivative of camptothecin which binds, along with its active metabolite SN-38, to the topoisomerase I-DNA complex. It is believed that cytotoxicity occurs as a result of irreparable double strand breaks caused by interaction of the topoisomerase I:DNA:irintecan or SN-38 ternary complex with replication enzymes. Irinotecan is indicated for treatment of metastatic cancer of the colon or rectum. The dose limiting side effects of irinotecan HCl are myelosuppression, including neutropenia, and GI effects, including diarrhea.

Topotecan HCl, (S)-10-[(dimethylamino)methyl]-4-ethyl-4,9-dihydroxy-1H-pyrano[3′,4′,6,7]indolizino[1,2-b]quinoline-3,14-(4H,12H)-dione monohydrochloride, is commercially available as the injectable solution HYCAMTIN®. Topotecan is a derivative of camptothecin which binds to the topoisomerase I-DNA complex and prevents religation of singles strand breaks caused by Topoisomerase I in response to torsional strain of the DNA molecule. Topotecan is indicated for second line treatment of metastatic carcinoma of the ovary and small cell lung cancer. The dose limiting side effect of topotecan HCl is myelosuppression, primarily neutropenia.

Also of interest, is the camptothecin derivative of formula A following, currently under development, including the racemic mixture (R,S) form as well as the R and S enantiomers:

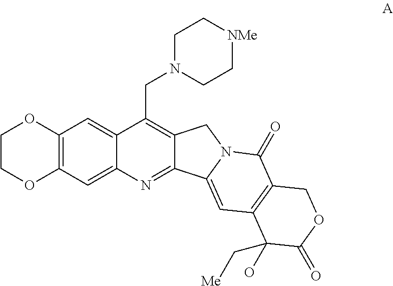 [SMILES: CCC1(O)C(=O)OCc2c1cc1n(c2=O)Cc2c-1nc1cc3c(cc1c2CN1CCN(C)CC1)OCCO3]

known by the chemical name “7-(4-methylpiperazino-methylene)-10,11-ethylenedioxy-20(R,S)-camptothecin (racemic mixture) or “7-(4-methylpiperazino-methylene)-10,11-ethylenedioxy-20(R)-camptothecin (R enantiomer) or “7-(4-methylpiperazino-methylene)-10,11-ethylenedioxy-20(S)-camptothecin (S enantiomer). Such compound as well as related compounds are described, including methods of making, in U.S. Pat. Nos. 6,063,923; 5,342,947; 5,559,235; 5,491,237 and pending U.S. patent application Ser. No. 08/977,217 filed Nov. 24, 1997.

Hormones and hormonal analogues are useful compounds for treating cancers in which there is a relationship between the hormone(s) and growth and/or lack of growth of the cancer. Examples of hormones and hormonal analogues useful in cancer treatment include, but are not limited to, adrenocorticosteroids such as prednisone and prednisolone which are useful in the treatment of malignant lymphoma and acute leukemia in children; aminoglutethimide and other aromatase inhibitors such as anastrozole, letrazole, vorazole, and exemestane useful in the treatment of adrenocortical carcinoma and hormone dependent breast carcinoma containing estrogen receptors; progestrins such as megestrol acetate useful in the treatment of hormone dependent breast cancer and endometrial carcinoma; estrogens, androgens, and anti-androgens such as flutamide, nilutamide, bicalutamide, cyproterone acetate and 5α-reductases such as finasteride and dutasteride, useful in the treatment of prostatic carcinoma and benign prostatic hypertrophy; anti-estrogens such as tamoxifen, toremifene, raloxifene, droloxifene, iodoxyfene, as well as selective estrogen receptor modulators (SERMS) such those described in U.S. Pat. Nos. 5,681,835, 5,877,219, and 6,207,716, useful in the treatment of hormone dependent breast carcinoma and other susceptible cancers; and gonadotropin-releasing hormone (GnRH) and analogues thereof which stimulate the release of leutinizing hormone (LH) and/or follicle stimulating hormone (FSH) for the treatment prostatic carcinoma, for instance, LHRH agonists and antagagonists such as goserelin acetate and luprolide.

Signal transduction pathway inhibitors are those inhibitors, which block or inhibit a chemical process which evokes an intracellular change. As used herein this change is cell proliferation or differentiation. Signal tranduction inhibitors useful in the present invention include inhibitors of receptor tyrosine kinases, non-receptor tyrosine kinases, SH2/SH3 domain blockers, serine/threonine kinases, phosphotidyl inositol-3 kinases, myo-inositol signaling, and Ras oncogenes.

Several protein tyrosine kinases catalyse the phosphorylation of specific tyrosyl residues in various proteins involved in the regulation of cell growth. Such protein tyrosine kinases can be broadly classified as receptor or non-receptor kinases.

Receptor tyrosine kinases are transmembrane proteins having an extracellular ligand binding domain, a transmembrane domain, and a tyrosine kinase domain. Receptor tyrosine kinases are involved in the regulation of cell growth and are generally termed growth factor receptors. Inappropriate or uncontrolled activation of many of these kinases, i.e. aberrant kinase growth factor receptor activity, for example by over-expression or mutation, has been shown to result in uncontrolled cell growth. Accordingly, the aberrant activity of such kinases has been linked to malignant tissue growth. Consequently, inhibitors of such kinases could provide cancer treatment methods. Growth factor receptors include, for example, epidermal growth factor receptor (EGFr), platelet derived growth factor receptor (PDGFr), erbB2, erbB4, vascular endothelial growth factor receptor (VEGFr), tyrosine kinase with immunoglobulin-like and epidermal growth factor homology domains (TIE-2), insulin growth factor-I (IGFI) receptor, macrophage colony stimulating factor (cfms), BTK, ckit, cmet, fibroblast growth factor (FGF) receptors, Trk receptors (TrkA, TrkB, and TrkC), ephrin (eph) receptors, and the RET protooncogene. Several inhibitors of growth receptors are under development and include ligand antagonists, antibodies, tyrosine kinase inhibitors and anti-sense oligonucleotides. Growth factor receptors and agents that inhibit growth factor receptor function are described, for instance, in Kath, John C., Exp. Opin. Ther. Patents (2000) 10(6):803-818; Shawver et al DDT Vol 2, No. 2 Feb. 1997; and Lofts, F. J. et al, “Growth factor receptors as targets”, New Molecular Targets for Cancer Chemotherapy, ed. Workman, Paul and Kerr, David, CRC press 1994, London.

Tyrosine kinases, which are not growth factor receptor kinases are termed non-receptor tyrosine kinases. Non-receptor tyrosine kinases useful in the present invention, which are targets or potential targets of anti-cancer drugs, include cSrc, Lck, Fyn, Yes, Jak, cAbl, FAK (Focal adhesion kinase), Brutons tyrosine kinase, and Bcr-Abl. Such non-receptor kinases and agents which inhibit non-receptor tyrosine kinase function are described in Sinh, S, and Corey, S. J., (1999) Journal of Hematotherapy and Stem Cell Research 8 (5): 465-80; and Bolen, J. B., Brugge, J. S., (1997) Annual review of Immunology. 15: 371-404.

SH2/SH3 domain blockers are agents that disrupt SH2 or SH3 domain binding in a variety of enzymes or adaptor proteins including, PI3-K p85 subunit, Src family kinases, adaptor molecules (Shc, Crk, Nck, Grb2) and Ras-GAP. SH2/SH3 domains as targets for anti-cancer drugs are discussed in Smithgall, T. E. (1995), Journal of Pharmacological and Toxicological Methods. 34(3) 125-32.

Inhibitors of Serine/Threonine Kinases including MAP kinase cascade blockers which include blockers of Raf kinases (rafk), Mitogen or Extracellular Regulated Kinase (MEKs), and Extracellular Regulated Kinases (ERKs); and Protein kinase C family member blockers including blockers of PKCs (alpha, beta, gamma, epsilon, mu, lambda, iota, zeta) IkB kinase family (IKKa, IKKb), PKB family kinases, AKT kinase family members, and TGF beta receptor kinases. Such Serine/Threonine kinases and inhibitors thereof are described in Yamamoto, T., Taya, S., Kaibuchi, K., (1999), Journal of Biochemistry. 126 (5) 799-803; Brodt, P, Samani, A., and Navab, R. (2000), Biochemical Pharmacology, 60. 1101-1107; Massague, J., Weis-Garcia, F. (1996) Cancer Surveys. 27:41-64; Philip, P. A., and Harris, A. L. (1995), Cancer Treatment and Research. 78: 3-27, Lackey, K. et al Bioorganic and Medicinal Chemistry Letters, (10), 2000, 223-226; U.S. Pat. No. 6,268,391; and Martinez-Iacaci, L., et al, Int. J. Cancer (2000), 88(1), 44-52.

Inhibitors of Phosphotidyl inositol-3 Kinase family members including blockers of PI3-kinase, ATM, DNA-PK, and Ku are also useful in the present invention. Such kinases are discussed in Abraham, R. T. (1996), Current Opinion in Immunology. 8 (3) 412-8; Canman, C. E., Lim, D. S. (1998), Oncogene 17 (25) 3301-3308; Jackson, S. P. (1997), International Journal of Biochemistry and Cell Biology. 29 (7):935-8; and Zhong, H. et al, Cancer res, (2000) 60(6), 1541-1545.

Also useful in the present invention are Myo-inositol signaling inhibitors such as phospholipase C blockers and Myoinositol analogues. Such signal inhibitors are described in Powis, G., and Kozikowski A., (1994) New Molecular Targets for Cancer Chemotherapy ed., Paul Workman and David Kerr, CRC press 1994, London.

Another group of signal transduction pathway inhibitors are inhibitors of Ras Oncogene. Such inhibitors include inhibitors of farnesyltransferase, geranyl-geranyl transferase, and CAAX proteases as well as anti-sense oligonucleotides, ribozymes and immunotherapy. Such inhibitors have been shown to block ras activation in cells containing wild type mutant ras, thereby acting as antiproliferation agents. Ras oncogene inhibition is discussed in Scharovsky, O. G., Rozados, V. R., Gervasoni, S. I. Matar, P. (2000), Journal of Biomedical Science. 7(4) 292-8; Ashby, M. N. (1998), Current Opinion in Lipidology. 9 (2) 99-102; and Bennett, C. F. and Cowsert, L. M. BioChim. Biophys. Acta, (1999) 1489(1):19-30.

As mentioned above, antibody antagonists to receptor kinase ligand binding may also serve as signal transduction inhibitors. This group of signal transduction pathway inhibitors includes the use of humanized antibodies to the extracellular ligand binding domain of receptor tyrosine kinases. For example Imclone C225 EGFR specific antibody (see Green, M. C. et al, Monoclonal Antibody Therapy for Solid Tumors, Cancer Treat. Rev., (2000), 26(4), 269-286); Herceptin® erbB2 antibody (see Tyrosine Kinase Signalling in Breast cancer:erbB Family Receptor Tyrosine Kniases, Breast cancer Res., 2000, 2(3), 176-183); and 2CB VEGFR2 specific antibody (see Brekken, R. A. et al, Selective Inhibition of VEGFR2Activity by a monoclonal Anti-VEGF antibody blocks tumor growth in mice, Cancer Res. (2000) 60, 5117-5124).

Non-receptor kinase angiogenesis inhibitors may also find use in the present invention. Inhibitors of angiogenesis related VEGFR and TIE2 are discussed above in regard to signal transduction inhibitors (both receptors are receptor tyrosine kinases). Angiogenesis in general is linked to erbB2/EGFR signaling since inhibitors of erbB2 and EGFR have been shown to inhibit angiogenesis, primarily VEGF expression. Thus, the combination of an erbB2/EGFR inhibitor with an inhibitor of angiogenesis makes sense. Accordingly, non-receptor tyrosine kinase inhibitors may be used in combination with the EGFR/erbB2 inhibitors of the present invention. For example, anti-VEGF antibodies, which do not recognize VEGFR (the receptor tyrosine kinase), but bind to the ligand; small molecule inhibitors of integrin (alphaᵥbeta₃) that will inhibit angiogenesis; endostatin and angiostatin (non-RTK) may also prove useful in combination with the disclosed erb family inhibitors. (See Bruns C J et al (2000), Cancer Res., 60: 2926-2935; Schreiber A B, Winkler M E, and Derynck R. (1986), Science, 232: 1250-1253; Yen L et al. (2000), Oncogene 19: 3460-3469).

Agents used in immunotherapeutic regimens may also be useful in combination with the compounds of formula (I). There are a number of immunologic strategies to generate an immune response against erbB2 or EGFR. These strategies are generally in the realm of tumor vaccinations. The efficacy of immunologic approaches may be greatly enhanced through combined inhibition of erbB2/EGFR signaling pathways using a small molecule inhibitor. Discussion of the immunologic/tumor vaccine approach against erbB2/EGFR are found in Reilly R T et al. (2000), Cancer Res. 60: 3569-3576; and Chen Y, Hu D, Eling D J, Robbins J, and Kipps T J. (1998), Cancer Res. 58: 1965-1971.

Agents used in proapoptotic regimens (e.g., bcl-2 antisense oligonucleotides) may also be used in the combination of the present invention. Members of the Bcl-2 family of proteins block apoptosis. Upregulation of bcl-2 has therefore been linked to chemoresistance. Studies have shown that the epidermal growth factor (EGF) stimulates anti-apoptotic members of the bcl-2 family (i.e., mcl-1). Therefore, strategies designed to downregulate the expression of bcl-2 in tumors have demonstrated clinical benefit and are now in Phase II/III trials, namely Genta's G3139 bcl-2 antisense oligonucleotide. Such proapoptotic strategies using the antisense oligonucleotide strategy for bcl-2 are discussed in Water J S et al. (2000), J. Clin. Oncol. 18: 1812-1823; and Kitada S et al. (1994), Antisense Res. Dev. 4: 71-79.

Cell cycle signalling inhibitors inhibit molecules involved in the control of the cell cycle. A family of protein kinases called cyclin dependent kinases (CDKs) and their interaction with a family of proteins termed cyclins controls progression through the eukaryotic cell cycle. The coordinate activation and inactivation of different cyclin/CDK complexes is necessary for normal progression through the cell cycle. Several inhibitors of cell cycle signalling are under development. For instance, examples of cyclin dependent kinases, including CDK2, CDK4, and CDK6 and inhibitors for the same are described in, for instance, Rosania et al, Exp. Opin. Ther. Patents (2000) 10(2):215-230.

In one embodiment, the cancer treatment method of the claimed invention includes the co-administration a compound of Formula (I) and/or a pharmaceutically acceptable salt, hydrate, solvate or pro-drug thereof and at least one anti-neoplastic agent, such as one selected from the group consisting of anti-microtubule agents, platinum coordination complexes, alkylating agents, antibiotic agents, topoisomerase II inhibitors, antimetabolites, topoisomerase I inhibitors, hormones and hormonal analogues, signal transduction pathway inhibitors, non-receptor tyrosine kinase angiogenesis inhibitors, immunotherapeutic agents, proapoptotic agents, and cell cycle signaling inhibitors.

### EXPERIMENTALS

Abbreviations: aq., aqueous; Boc, t-butyloxycarbonyl; CH₂Cl₂, dichloromethane; CH₃CN, acetonitrile; Cp*RuCl(PPh₃)₂, pentamethylcyclopentadienylbis(triphenylphosphine)-ruthenium(II) chloride; DCE, 1,2-dichloroethane; DIAD, diisopropyl azodicarboxylate; DIPEA, diisopropylethylamine; DMAP, 4-N,N-dimethylaminopyridine; DME, 1,2-dimethoxyethane; DMF, N,N-dimethylformamide; DMSO, dimethylsulfoxide; EDC, N-(3-dimethylaminopropyl)-N-ethylcarbodiimide hydrochloride; Et₃N, triethylamine; Et₂O, diethyl ether; EtOAc, ethyl acetate; EtOH, ethanol; HCl, hydrochloric acid; HOAc, acetic acid; HOAt, 1-hydroxy-7-azabenzotriazole; HOBt, 1-hydroxybenzotriazole; K₂CO₃, potassium carbonate; KOAc, potassium acetate; MeOH, methanol; MgSO₄, magnesium sulfate; MsCl, methanesulfonyl chloride; NaCN, sodium cyanide; Na₂CO₃, sodium carbonate; NaH, sodium hydride; NaHCO₃, sodium bicarbonate; NaN₃, sodium azide; Na₂SO₄, sodium sulfate; NH₄Cl, ammonium chloride; NH₄OH, ammonium hydroxide; PdCl₂(dppf), 1,1′-bis(diphenylphosphino)ferrocene-palladium(II)dichloride•dichloromethane complex; Pd(OAc)₂, palladium(II) acetate; Pd(PPh₃)₄, tetrakis(triphenylphosphine)palladium(0); PPh₃, triphenylphosphine; THF, tetrahydrofuran; TFA, trifluoroacetic acid; TMSN₃, trimethylsilyl azide.

### Preparation

The derivatives described herein were prepared by the general methods described below:

### Schemes/Experimentals

Substituted tetrazoles can be prepared as outlined in Scheme I. Substituted amides can be prepared from a pyrrolidine or azetidine methylamine, such as the commercially available 1,1-dimethylethyl 3-(aminomethyl)-1-pyrrolidinecarboxylate, 1,1-dimethylethyl (3S)-3-(aminomethyl)-1-pyrrolidinecarboxylate, or N-Boc-4-methylamineazetidine, and a functionalized benzoic acid, and this can then be converted into the corresponding tetrazole (J. Org. Chem. 1991, 56, 2395-2400). Deprotection and then acylation of the pyrrolidine followed by a Suzuki coupling can then provide the desired tetrazoles.

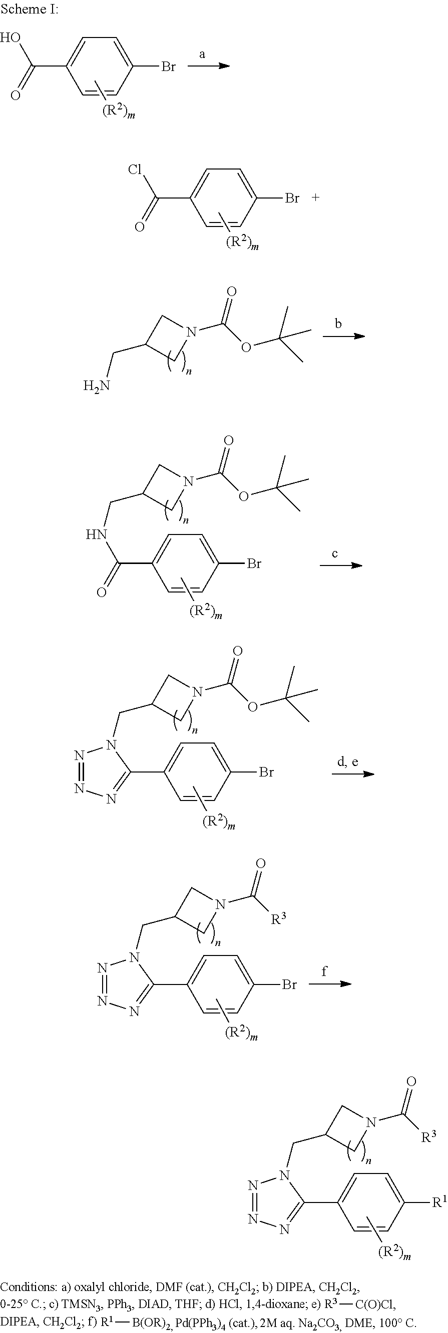

Substituted 1,2,3-triazoles can be prepared as outlined in Scheme II, starting from a pyrrolidine or azetidine methyl alcohol, such as the commercially available 1,1-dimethylethyl 3-(hydroxymethyl)-1-pyrrolidinecarboxylate, 1,1-dimethylethyl (3R)-3-(hydroxymethyl)-1-pyrrolidinecarboxylate, or 1,1-dimethylethyl 3-(hydroxymethyl)-1-azetidinecarboxylate. Formation of the azide followed by Ru-catalyzed cyclization with a functionalized alkyne can provide the 1,2,3-triazole framework. Deprotection and acylation of the pyrrolidine followed by a Suzuki coupling can then provide the desired triazoles.

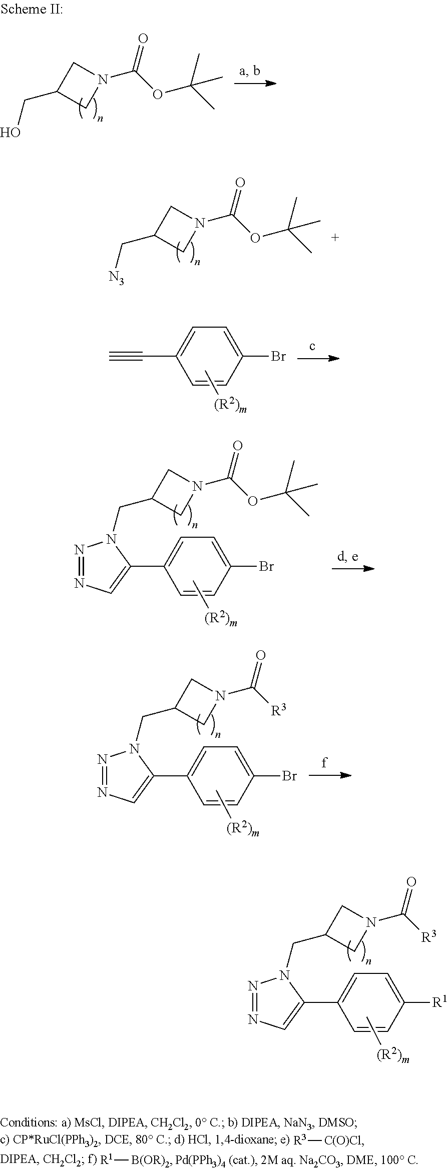

Substituted 1,2,4-triazoles can be prepared as outlined in Scheme III. Condensation of a functionalized benzohydrazide with an appropriate acetal and then cyclization with a pyrrolidine or azetidine methylamine can give a functionalized 1,2,4-triazole. Further manipulation through deprotection and acylation of the pyrrolidine and Suzuki coupling can then provide the desired triazoles.

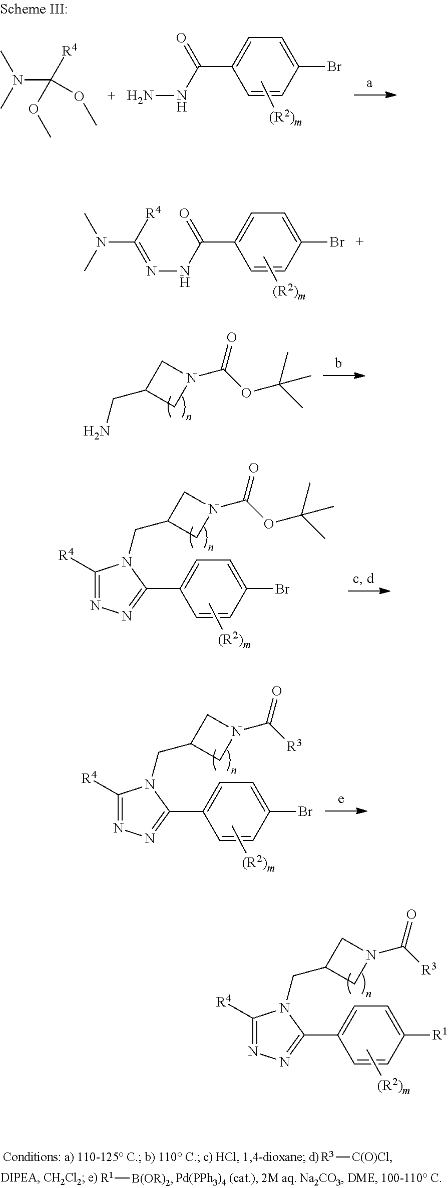

Substituted imidazoles can be prepared as outlined in Scheme IV through alkylation of a functionalized aryl imidazole. Subsequent deprotection and acylation of the pyrrolidine followed by Suzuki coupling can then provide the desired imidazoles.

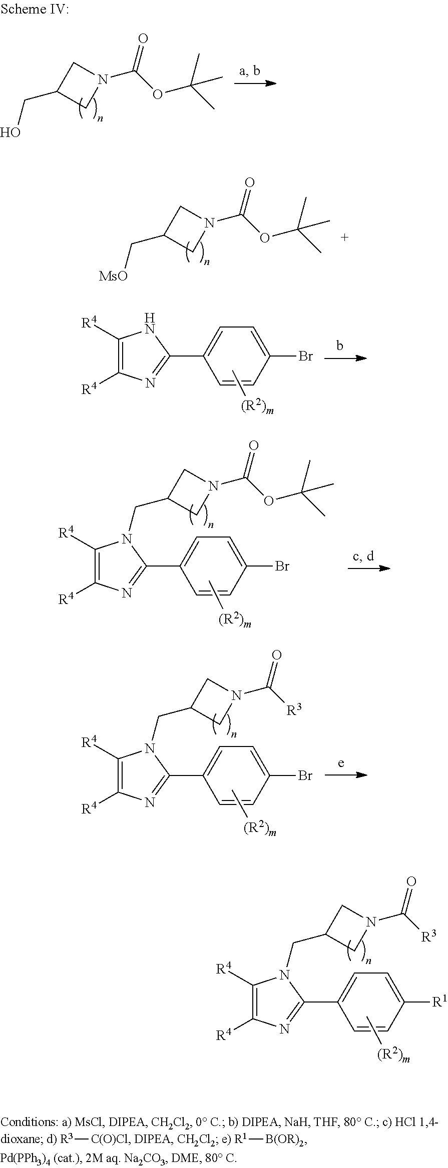

### Example 1

### 6-[4-(1-{[(3R)-1-(cyclopropylcarbonyl)-3-pyrrolidinyl]methyl}-1H-tetrazol-5-yl)phenyl]-1H-indole

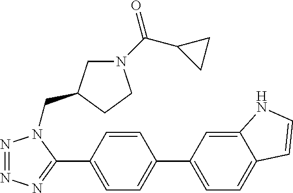 [SMILES: O=C(C1CC1)N1CC[C@@H](Cn2nnnc2-c2ccc(-c3ccc4cc[nH]c4c3)cc2)C1]

### a) 1,1-dimethylethyl (3S)-3-({[(4-bromophenyl)carbonyl]amino}methyl)-1-pyrrolidinecarboxylate

To a suspension of 4-bromobenzoic acid (3.01 g, 14.97 mmol) in CH₂Cl₂ (100 mL) was added oxalyl chloride (6.55 mL, 74.9 mmol) and 1 drop of DMF and the reaction mixture was stirred at 25° C. for 2 h, at which time carbon dioxide evolution had ceased and a solution had been obtained. The reaction mixture was concentrated in vacuo under high vacuum to remove residual oxalyl chloride. The crystalline residue was dissolved in CH₂Cl₂ (100 mL), cooled in an ice bath, and treated successively with 1,1-dimethylethyl (3S)-3-(aminomethyl)-1-pyrrolidinecarboxylate (3.00 g, 14.97 mmol) and DIPEA (7.85 mL, 44.9 mmol). The reaction mixture was stirred for 1 h at ice-bath temperature and then allowed to warm to ambient temperature and was stirred overnight. The reaction mixture was concentrated to dryness, dissolved in Et₂O and a fine white solid was filtered off. The filtrate was concentrated to dryness and purified on silica gel eluted with 20 to 70%

EtOAc/hexanes to give the title compound (4.63 g, 81%) as a white foam. ¹H NMR (400 MHz, DMSO-d₆) δ ppm 8.64 (d, J=2.84 Hz, 1H), 7.73-7.81 (m, 2H), 7.63-7.69 (m, 2 H), 3.06-3.44 (m, 5H), 2.87-3.04 (m, 1H), 2.30-2.46 (m, 1H), 1.78-1.95 (m, 1H), 1.47-1.69 (m, 1H), 1.36 (s, 9H). LCMS (ES⁺) m/z 382.93, 384.94 [M+H]⁺.

### b) 1,1-dimethylethyl (3R)-3-{[5-(4-bromophenyl)-1H-tetrazol-1-yl]methyl}-1-pyrrolidinecarboxylate

1,1-dimethylethyl (3S)-3-({[(4-bromophenyl)carbonyl]amino}methyl)-1-pyrrolidinecarboxylate (2.08 g, 5.43 mmol) and PPh₃ (2.85 g, 10.85 mmol) were dissolved in dry THF (100 mL) and treated successively with DIAD (2.245 mL, 10.85 mmol) and TMSN₃ (1.485 mL, 10.85 mmol) with stirring at 25° C. After stirring overnight, analysis of the reaction mixture by LCMS indicated ˜20% conversion. Stirring was continued an additional 24 h after which time reaction progress appeared to have stopped. Additional DIAD (1.055 mL, 5.43 mmol) dissolved in THF (10 mL) was added dropwise and the reaction mixture was stirred an additional 24 h. Additional TMSN₃ (0.720 mL, 5.43 mmol) dissolved in THF (10 mL) was added dropwise and the reaction mixture was stirred an additional 3 days. Analysis by LCMS indicated no further reaction progress. The reaction mixture was concentrated to dryness and partitioned between Et₂O and saturated aq. NaHCO₃. The organic phase was isolated, dried over MgSO₄, filtered and concentrated in vacuo. The residue was purified by flash chromatography with an eluent of 20 to 100% EtOAc/hexanes to give the title compound (1.15 g, 51.9%). LCMS (ES⁺) m/z 407.95, 409.94 [M+H]⁺. ¹H NMR (400 MHz, CDCl₃) δ ppm 7.74 (d, J=8.42 Hz, 2H), 7.50-7.57 (m, 2H), 4.34-4.48 (m, 2H), 3.23-3.58 (m, 3H), 2.99-3.12 (m, 1H), 2.75-2.89 (m, 1 H), 1.93-2.04 (m, 1H), 1.57-1.70 (m, 1H), 1.44 (s, 9H).

### c) 5-(4-bromophenyl)-1-[(3R)-3-pyrrolidinylmethyl]-1H-tetrazole

1,1-dimethylethyl (3R)-3-{[5-(4-bromophenyl)-1H-tetrazol-1-yl]methyl}-1-pyrrolidinecarboxylate (1.15 g, 2.82 mmol) was dissolved in a solution of 4 N HCl in 1,4-dioxane (7 mL, 28.0 mmol) and stirred at 25° C. An oily material quickly separated from the reaction mixture which after continued stirring for 30 min yielded a white solid. This material was filtered off, washed repeatedly with Et₂O, and air dried to give the HCl salt of the title compound (0.792 g, 82%) as a white solid. LCMS (ES⁺) m/z 307.91, 309.90 [M+H]⁺. ¹H NMR (400 MHz, DMSO-d₆) δ ppm 8.87-9.23 (m, 2H), 7.82-7.89 (m, 2H), 7.73-7.80 (m, 2H), 4.52-4.68 (m, 2H), 3.26-3.36 (m, 1H), 3.16-3.26 (m, 1H), 3.01-3.15 (m, 1H), 2.78-2.99 (m, 2H), 1.93-2.06 (m, 1H), 1.51-1.66 (m, 1H) (HCl peak apparent in the 8.87-9.23 peak).

### d) 5-(4-bromophenyl)-1-{[(3R)-1-(cyclopropylcarbonyl)-3-pyrrolidinyl]methyl}-1H-tetrazole

To a slurry of 5-(4-bromophenyl)-1-[(3R)-3-pyrrolidinylmethyl]-1H-tetrazole hydrochloride (0.500 g, 1.451 mmol) and DIPEA (0.760 mL, 4.35 mmol) in CH₂Cl₂ (10 mL) was added cyclopropanecarbonyl chloride (0.182 g, 1.741 mmol) at 25° C. and the mixture was stirred for 1 h. The reaction mixture was diluted with CH₂Cl₂ and washed successively with saturated aq. NaHCO₃ and dilute aq. HCl. The organic phase was isolated, dried over MgSO₄, filtered and concentrated in vacuo. The residue was purified by flash chromatography with an eluent of 100% EtOAc to 10% MeOH/CH₂Cl₂ to give the title compound (0.513 g, 94%) as a clear oil. LCMS (ES⁺) m/z 375.93, 377.91 [M+H]⁺. ¹H NMR (400 MHz, CDCl₃) δ ppm 7.70-7.77 (m, 2H), 7.50-7.57 (m, 2H), 4.34-4.54 (m, 2H), 3.13-3.88 (m, 4H), 2.76-3.01 (m, 1H), 2.00-2.18 (m, 1H), 1.45-1.85 (m, 2H), 0.88-1.04 (m, 2H), 0.70-0.81 (m, 2H).

### e) 6-[4-(1-{[(3R)-1-(cyclopropylcarbonyl)-3-pyrrolidinyl]methyl}-1H-tetrazol-5-yl)phenyl]-1H-indole

5-(4-bromophenyl)-1-{[(3R)-1-(cyclopropylcarbonyl)-3-pyrrolidinyl]methyl}-1H-tetrazole (0.100 g, 0.266 mmol), 1H-indol-6-ylboronic acid (0.086 g, 0.532 mmol), and Pd(PPh₃)₄ (0.015 g, 0.013 mmol) were combined in DME (4 mL) and 2 M aq. Na₂CO₃ (4 mL), purged with nitrogen, and irradiated in a microwave reactor at 100° C. for 45 min. The reaction mixture was partitioned between EtOAc and water and the organic phase was isolated, dried over MgSO₄, filtered and concentrated in vacuo. The residue was purified by flash chromatography using an eluent of EtOAc to give the title compound (0.101 g, 92%) as a white foam. LCMS (ES⁻) m/z 411.26 [M]. LCMS (ES⁺) m/z 413.06 [M+H]⁺. ¹H NMR (400 MHz, DMSO-d₆) δ ppm 11.28 (br. s, 1H), 7.86-7.97 (m, 4H), 7.76 (s, 1H), 7.67 (d, J=8.32 Hz, 1H), 7.39-7.46 (m, 2H), 6.48 (t, J=1.96 Hz, 1H), 4.63 (t, J=7.83 Hz, 2H), 3.13-3.82 (m, 4H), 2.70-2.93 (m, 1H), 1.87-2.11 (m, 1H), 1.57-1.81 (m, 2 H), 0.63-0.72 (m, 4H).

### Example 2

### 5-[4-(1-benzofuran-5-yl)phenyl]-1-{[(3R)-1-(cyclopropylcarbonyl)-3-pyrrolidinyl]methyl}-1H-tetrazole

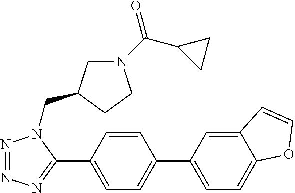 [SMILES: O=C(C1CC1)N1CC[C@@H](Cn2nnnc2-c2ccc(-c3ccc4occc4c3)cc2)C1]

5-(4-bromophenyl)-1-{[(3R)-1-(cyclopropylcarbonyl)-3-pyrrolidinyl]methyl}-1H-tetrazole (0.133 mmol), 5-(4,4,5,5-tetramethyl-1,3,2-dioxaborolan-2-yl)-1-benzofuran (0.133 mmol), and Pd(PPh₃)₄ (0.0066 mmol) were combined in CH₃CN (2 mL) and 5% aq. Na₂CO₃ (2 mL), purged with nitrogen, and heated at 90° C. for 2 h. The reaction mixture was partitioned between EtOAc and water and the organic phase was then washed with brine and concentrated in vacuo. The residue was purified by reverse phase HPLC (25-99% CH₃CN/water with 0.1% TFA) and the title compound was collected, concentrated in vacuo, and lyophilized to afford a white solid (0.041 g, 74%). LCMS (ES⁺) m/z 414.0 [M+H]⁺.

### Example 3

### 5-[4-(1-{[(3R)-1-(cyclopropylcarbonyl)-3-pyrrolidinyl]methyl}-1H-tetrazol-5-yl)phenyl]-1H-indole

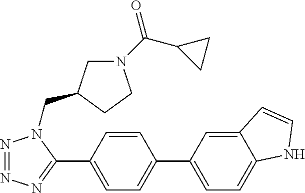 [SMILES: O=C(C1CC1)N1CC[C@@H](Cn2nnnc2-c2ccc(-c3ccc4[nH]ccc4c3)cc2)C1]

Following the procedure described in Example 2a with 1H-indol-5-ylboronic acid and heating for 16 h gave the product contaminated by some triphenylphosphine oxide. Additional purification of the product by reverse phase HPLC (30-60% CH₃CN/water with 0.1% TFA) provided the trifluoroacetate salt of the title compound (0.027 g, 37%) as a white solid. LCMS (ES⁺) m/z 413.1 [M+H]⁺.

### Example 4

### 1-{[(3R)-1-(cyclopropylcarbonyl)-3-pyrrolidinyl]methyl}-5-(2′,4′-dichloro-4-biphenylyl)-1H-tetrazole

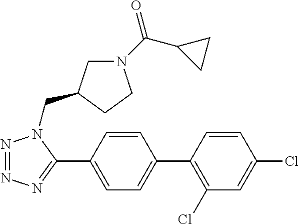 [SMILES: O=C(C1CC1)N1CC[C@@H](Cn2nnnc2-c2ccc(-c3ccc(Cl)cc3Cl)cc2)C1]

Following the procedure described in Example 2a with (2,4-dichlorophenyl)boronic acid and heating overnight resulted in an incomplete reaction. Additional (2,4-dichlorophenyl)boronic acid (0.067 mmol) and palladium catalyst (0.0033 mmol) were added and the reaction mixture was heated at 90° C. Work-up and purification of the product as described in Example 2a afforded the trifluoroacetate salt of the title compound (0.044 g, 59%) as a white solid. LCMS (ES⁺) m/z 441.9 [M+H]⁺.

### Example 5

### 5-[2′-chloro-4′-(methyloxy)-4-biphenylyl]-1-{[(3R)-1-(cyclopropylcarbonyl)-3-pyrrolidinyl]methyl}-1H-tetrazole

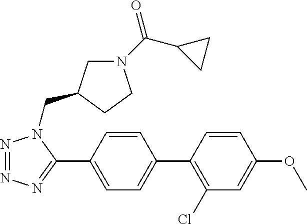 [SMILES: COc1ccc(-c2ccc(-c3nnnn3C[C@@H]3CCN(C(=O)C4CC4)C3)cc2)c(Cl)c1]

Following the procedure described in Example 2a with [2-chloro-4-(methyloxy)phenyl]boronic acid resulted in an incomplete reaction. Additional [2-chloro-4-(methyloxy)phenyl]boronic acid (2×0.133 mmol) and palladium catalyst (2×0.0066 mmol) were added and the reaction mixture was heated for prolonged lengths of time, but this resulted in only a modest progression of the reaction. Work-up and purification of the product as described in Example 2a afforded the title compound (0.011 g, 19%) as a white solid. LCMS (ES⁺) m/z 438.0 [M+H]⁺.

### Example 6

### 1-{[(3R)-1-(cyclopropylcarbonyl)-3-pyrrolidinyl]methyl}-5-(4′-fluoro-4-biphenylyl)-1H-tetrazole

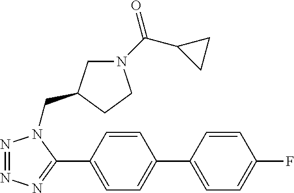 [SMILES: O=C(C1CC1)N1CC[C@@H](Cn2nnnc2-c2ccc(-c3ccc(F)cc3)cc2)C1]

Following the procedure described in Example 2a with (4-fluorophenyl)boronic acid and purification of the residue by reverse phase HPLC (10-70% CH₃CN/water with 0.1% TFA) afforded the trifluoroacetate salt of the title compound (0.036 g, 54%) as a white solid. LCMS (ES⁺) m/z 392.0 [M+H]⁺.

### Example 7

### 6-[4-(1-{[(3R)-1-(cyclopropylcarbonyl)-3-pyrrolidinyl]methyl}-1H-tetrazol-5-yl)phenyl]-1H-indazole

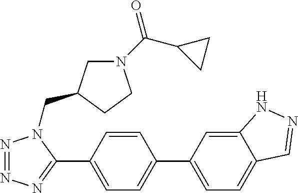 [SMILES: O=C(C1CC1)N1CC[C@@H](Cn2nnnc2-c2ccc(-c3ccc4cn[nH]c4c3)cc2)C1]

### a) 1,1-dimethylethyl 6-bromo-1H-indazole-1-carboxylate

A suspension of 6-bromo-1H-indazole (82.74 mmol), DMAP (16.55 mmol), and Et₃N (19.56 mL) in CH₃CN at 0° C. was treated with bis(1,1-dimethylethyl) dicarbonate (82.74 mmol) in CH₃CN over 15 min such that the internal temperature remained at 5° C. The reaction mixture was warmed to room temperature and stirred for 18 h. The reaction was concentrated in vacuo and the residue purified by flash chromatography (7% EtOAc/petroleum ether) to afford the title compound (23.2 g, 94%) as a solid.

### b) 1,1-dimethylethyl 6-(4,4,5,5-tetramethyl-1,3,2-dioxaborolan-2-yl)-1H-indazole-1-carboxylate

To a solution of 1,1-dimethylethyl 6-bromo-1H-indazole-1-carboxylate (10.1 mmol) in 1,4-dioxane (60 mL) was added 4,4,4′,4′,5,5,5′,5′-octamethyl-2,2′-bi-1,3,2-dioxaborolane (11.1 mmol), KOAc (30.3 mmol), Et₃N (1.5 mL), Pd(OAc)₂ (1.01 mmol), and PdCl₂(dppf) (1.01 mmol). The reaction mixture was stirred at 110° C. overnight and then concentrated in vacuo. The residue was purified by flash chromatography (5-10% EtOAc/petroleum ether) to yield the title compound (1.61 g, 46%) as a solid.

### c) 6-[4-(1-{[(3R)-1-(cyclopropylcarbonyl)-3-pyrrolidinyl]methyl}-1H-tetrazol-5-yl)phenyl]-1H-indazole

Following the procedure described in Example 6a with 1,1-dimethylethyl 6-(4,4,5,5-tetramethyl-1,3,2-dioxaborolan-2-yl)-1H-indazole-1-carboxylate provided the trifluoroacetate salt of the title compound (0.036 g, 34%) as a white solid. LCMS (ES⁺) m/z 414.0 [M+H]⁺.

### Example 8

### 6-[4-(1-{[(3R)-1-(cyclopropylcarbonyl)-3-pyrrolidinyl]methyl}-1H-1,2,3-triazol-5-yl)phenyl]-1H-indole

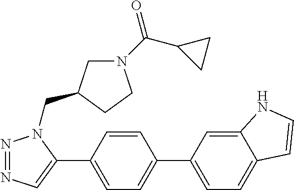 [SMILES: O=C(C1CC1)N1CC[C@@H](Cn2nncc2-c2ccc(-c3ccc4cc[nH]c4c3)cc2)C1]

### a) 1,1-dimethylethyl (3R)-3-{[(methylsulfonyl)oxy]methyl}-1-pyrrolidinecarboxylate

1,1-dimethylethyl (3R)-3-(hydroxymethyl)-1-pyrrolidinecarboxylate (3.78 g, 18.78 mmol) and DIPEA (6.56 mL, 37.6 mmol) dissolved in CH₂Cl₂ (40 mL) at 0° C. was treated dropwise with MsCl (1.537 mL, 19.72 mmol) dissolved in CH₂Cl₂ (20 mL). After 1 h, the reaction mixture was washed with water and dilute aq. HCl, dried over MgSO₄, filtered, and concentrated in vacuo to provide an amber oil. The residue was purified via flash chromatography (20-80% EtOAc/hexanes) to give the title compound. ¹H NMR (400 MHz, CDCl₃) δ ppm 4.11-4.28 (m, 2H), 3.56 (dd, J=11.11, 7.49 Hz, 1H), 3.42-3.52 (m, 1H), 3.36 (dt, J=10.82, 7.66 Hz, 1H), 3.15 (m, 1H), 3.04 (s, 3H), 2.64 (m, 1H), 1.99-2.11 (m, 1H), 1.69-1.81 (m, 1H), 1.47 (s, 9H).

### b) 1,1-dimethylethyl (3R)-3-(azidomethyl)-1-pyrrolidinecarboxylate

1,1-dimethylethyl (3R)-3-{[(methylsulfonyl)oxy]methyl}-1-pyrrolidinecarboxylate (1.50 g, 5.37 mmol) and DIPEA (0.938 mL, 5.37 mmol) in dimethylsulfoxide (5 mL) was treated with NaN₃ (0.349 g, 5.37 mmol) and stirred for four days. The reaction mixture was diluted with EtOAc, washed four times with water, dried over MgSO₄, filtered, and concentrated in vacuo to a clear oil. The residue was purified by flash chromatography (20-80% EtOAc/hexanes) to give the title compound (0.44 g, 36%). LCMS (ES⁺) m/z 227.08 [M+H]⁺. ¹H NMR (400 MHz, CDCl₃) δ ppm 3.53 (dd, J=10.96, 7.44 Hz, 1H), 3.46 (ddd, J=10.96, 8.12, 4.40 Hz, 1H), 3.26-3.38 (m, 3H), 3.06 (dd, J=10.96, 7.24 Hz, 1 H), 2.36-2.51 (m, 1H), 1.97-2.08 (m, 1H), 1.60-1.69 (m, 1H), 1.46 (s, 9H).

### c) 1,1-dimethylethyl (3R)-3-{[5-(4-bromophenyl)-1H-1,2,3-triazol-1-yl]methyl}-1-pyrrolidinecarboxylate

1,1-dimethylethyl (3R)-3-(azidomethyl)-1-pyrrolidinecarboxylate (0.275 g, 1.215 mmol) and 1-bromo-4-ethynylbenzene (0.264 g, 1.458 mmol) in DCE (10 mL) was treated with Cp*RuCl(PPh₃)₂ (0.048 g, 0.061 mmol) and the reaction mixture was stirred at 80° C. for 8 h, after which time analysis by TLC indicated complete conversion to product. The reaction mixture was concentrated in vacuo and the residue was purified on by flash chromatography (20-70% EtOAc/hexanes) to give the title compound (0.193 g, 39%). LCMS (ES⁺) m/z 406.95, 408.90 [M+H]⁺. ¹H NMR (400 MHz, CDCl₃) δ ppm 7.70 (br. s., 1H), 7.65 (d, J=8.32 Hz, 2H), 7.24 (d, J=8.52 Hz, 2H), 4.28-4.37 (m, 2H), 3.19-3.48 (m, 3H), 2.99 (dd, J=11.11, 7.00 Hz, 1H), 2.65-2.82 (m, 1H), 1.84-1.96 (m, 1H), 1.51-1.63 (m, 1H), 1.43 (s, 9H).

### d) 5-(4-bromophenyl)-1-[(3R)-3-pyrrolidinylmethyl]-1H-1,2,3-triazole

1,1-dimethylethyl (3R)-3-{[5-(4-bromophenyl)-1H-1,2,3-triazol-1-yl]methyl}-1-pyrrolidinecarboxylate (0.193 g, 0.474 mmol) was treated with 4 N HCl in 1,4-dioxane (5 mL, 20.00 mmol) at 25° C. with stirring for 2 h. The resulting precipitate was filtered off, washed with Et₂O, and dried via air suction to give the title compound as a sticky tan solid. LCMS (ES⁺) m/z 306.92, 308.90 [M+H]⁺. ¹H NMR (400 MHz, DMSO-d₆) δ ppm 8.95-9.22 (m, 2H), 7.95 (s, 1H), 7.71-7.80 (m, 2H), 7.50-7.58 (m, 2H), 4.43-4.59 (m, 2H), 3.11-3.29 (m, 2H), 2.98-3.11 (m, 1H), 2.81-2.96 (m, 1H), 2.63-2.81 (m, 1H), 1.83-1.97 (m, 1H), 1.52 (m, 1H).

### e) 5-(4-bromophenyl)-1-{[(3R)-1-(cyclopropylcarbonyl)-3-pyrrolidinyl]methyl}-1H-1,2,3-triazole

To a slurry of 5-(4-bromophenyl)-1-[(3R)-3-pyrrolidinylmethyl]-1H-1,2,3-triazole (0.163 g, 0.474 mmol) and DIPEA (0.248 mL, 1.422 mmol) in CH₂Cl₂ (5 mL) at 25° C. was added cyclopropanecarbonyl chloride (0.059 g, 0.569 mmol). After 1 h, the reaction mixture was diluted with CH₂Cl₂ and washed successively with saturated aq. NaHCO₃ and dilute aq. HCl. The organic phase was isolated, dried over MgSO₄, filtered, and concentrated in vacuo. The residue was purified via flash chromatography (EtOAc to 10% MeOH/CH₂Cl₂) to give the title compound (0.155 g, 87% (2 steps)) as a clear oil. LCMS (ES⁺) m/z 374.93, 376.88 [M+H]⁺. ¹H NMR (400 MHz, DMSO-d₆) δ ppm 7.93 (d, J=2.06 Hz, 1H), 7.72-7.78 (m, 2H), 7.51-7.57 (m, 2H), 4.46 (t, J=7.63 Hz, 2H), 2.90-3.70 (m, 4H), 2.52-2.76 (m, 1H), 1.75-1.97 (m, 1H), 1.43-1.70 (m, 2H), 0.67 (d, J=5.97 Hz, 4H).

### f) 6-[4-(1-{[(3R)-1-(cyclopropylcarbonyl)-3-pyrrolidinyl]methyl}-1H-1,2,3-triazol-5-yl)phenyl]-1H-indole

5-(4-bromophenyl)-1-{[(3R)-1-(cyclopropylcarbonyl)-3-pyrrolidinyl]methyl}-1H-1,2,3-triazole (0.060 g, 0.160 mmol), 1H-indol-6-ylboronic acid (0.051 g, 0.320 mmol), and Pd(PPh₃)₄ (9.24 mg, 7.99 μmol) were combined in DME (2 mL) and 2 M aq. Na₂CO₃ (2 mL), purged with nitrogen, and irradiated in a microwave reactor at 100° C. for 45 min. The reaction mixture was partitioned between EtOAc and water and the organic phase was isolated, dried over MgSO₄, filtered, and concentrated in vacuo. The residue was purified by flash chromatography (EtOAc followed by a gradient of 100% EtOAc to 10% MeOH/CH₂Cl₂) to afford the title compound (0.0520 g, 79%) as a tan foam. LCMS (ES⁻) m/z 410.17 [M-1]. LCMS (ES⁺) m/z 412.05 [M+H]⁺. ¹H NMR (400 MHz, DMSO-d₆) δ ppm 11.25 (br. s., 1H), 7.95 (d, J=2.35 Hz, 1H), 7.85 (dd, J=8.37, 2.01 Hz, 2H), 7.72 (s, 1H), 7.60-7.69 (m, 3H), 7.36-7.44 (m, 2H), 6.47 (d, J=1.96 Hz, 1H), 4.53 (dd, J=10.62, 7.49 Hz, 2H), 2.98-3.72 (m, 4H), 2.58-2.83 (m, 1H), 1.80-2.02 (m, 1H), 1.50-1.74 (m, 2H), 0.60-0.72 (m, 4H).

### Example 9

### 5-[4-(1-{[(3R)-1-(cyclopropylcarbonyl)-3-pyrrolidinyl]methyl}-1H-1,2,3-triazol-5-yl)phenyl]-1H-indole

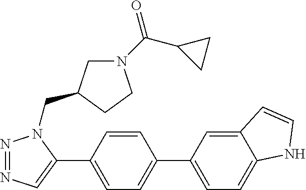 [SMILES: O=C(C1CC1)N1CC[C@@H](Cn2nncc2-c2ccc(-c3ccc4[nH]ccc4c3)cc2)C1]

Following the procedure described in Example 8f with 1H-indol-5-ylboronic acid and heating in an oil bath at 100° C. for 2 h afforded the crude product, which was purified by flash chromatography (0-10% MeOH/CH₂Cl₂), dissolved in EtOAc and precipitated with hexanes, filtered, and rinsed with hexanes to afford the title compound (0.071 g, 72%) as a white solid. LCMS (ES⁺) m/z 412.4 [M+H]⁺.

### Example 10

### 5-[4-(1-benzofuran-5-yl)phenyl]-1-{[(3R)-1-(cyclopropylcarbonyl)-3-pyrrolidinyl]methyl}-1H-1,2,3-triazole

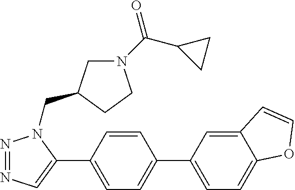 [SMILES: O=C(C1CC1)N1CC[C@@H](Cn2nncc2-c2ccc(-c3ccc4occc4c3)cc2)C1]

Following the procedure described in Example 9a with 5-(4,4,5,5-tetramethyl-1,3,2-dioxaborolan-2-yl)-1-benzofuran provided the title compound (0.091 g, 98%) as a tan foam after purification by flash chromatography. LCMS (ES⁺) m/z 413.4 [M+H]⁺.

### Example 11

### 5-[4-(1-{[(3R)-1-(cyclopropylcarbonyl)-3-pyrrolidinyl]methyl}-1H-1,2,3-triazol-5-yl)phenyl]-1H-indazole

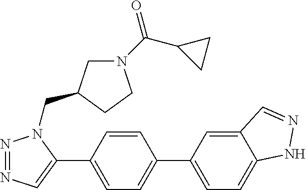 [SMILES: O=C(C1CC1)N1CC[C@@H](Cn2nncc2-c2ccc(-c3ccc4[nH]ncc4c3)cc2)C1]

### a) 1,1-dimethylethyl 5-bromo-1H-indazole-1-carboxylate

Following the procedure described in Example 7a with 5-bromo-1H-indazole provided the title compound.

### b) 1,1-dimethylethyl 5-(4,4,5,5-tetramethyl-1,3,2-dioxaborolan-2-yl)-1H-indazole-1-carboxylate

Following the procedure described in Example 7b with 1,1-dimethylethyl 5-bromo-1H-indazole-1-carboxylate and purification by flash chromatography using 5% EtOAc/petroleum ether gave the title compound (74%) as a yellow solid.

### c) 5-[4-(1-{[(3R)-1-(cyclopropylcarbonyl)-3-pyrrolidinyl]methyl}-1H-1,2,3-triazol-5-yl)phenyl]-1H-indazole

Following the procedure described in Example 9a with 1,1-dimethylethyl 5-(4,4,5,5-tetramethyl-1,3,2-dioxaborolan-2-yl)-1H-indazole-1-carboxylate provided the title compound (0.071 g, 72%) as an ivory solid. LCMS (ES⁺) m/z 413.4 [M+H]⁺.

### Example 12

### 1-{[(3R)-1-(cyclopropylcarbonyl)-3-pyrrolidinyl]methyl}-5-(2′,4′-dichloro-4-biphenylyl)-1H-1,2,3-triazole

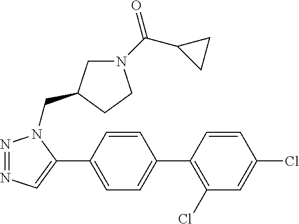 [SMILES: O=C(C1CC1)N1CC[C@@H](Cn2nncc2-c2ccc(-c3ccc(Cl)cc3Cl)cc2)C1]

Following the procedure described in Example 9a with (2,4-dichlorophenyl)boronic acid provided the title compound (0.084 g, 79%) as a white foam. LCMS (ES⁺) m/z 441.4, 443.9 [M+H]⁺.

### Example 13

### 5-[2′-chloro-4′-(methyloxy)-4-biphenylyl]-1-{[(3R)-1-(cyclopropylcarbonyl)-3-pyrrolidinyl]methyl}-1H-1,2,3-triazole

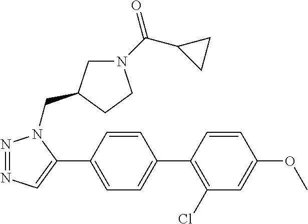 [SMILES: COc1ccc(-c2ccc(-c3cnnn3C[C@@H]3CCN(C(=O)C4CC4)C3)cc2)c(Cl)c1]

Following the procedure described in Example 10a with [2-chloro-4-(methyloxy)phenyl]boronic acid provided the title compound (0.080 g, 76%) as a colorless foam. LCMS (ES⁺) m/z 437.4 [M+H]⁺.

### Example 14

### 6-[4-(4-{[(3S)-1-(cyclopropylcarbonyl)-3-pyrrolidinyl]methyl}-4H-1,2,4-triazol-3-yl)phenyl]-1H-indole

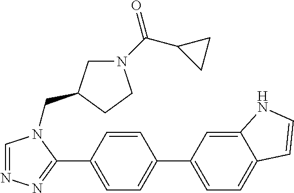 [SMILES: O=C(C1CC1)N1CC[C@@H](Cn2cnnc2-c2ccc(-c3ccc4cc[nH]c4c3)cc2)C1]

### a) N′-[(4-bromophenyl)carbonyl]-N,N-dimethylhydrazonoformamide

4-bromobenzohydrazide (5.15 g, 23.95 mmol) and N,N-dimethylformamide dimethylacetal (50 mL, 373 mmol) were combined in a round bottom flask and stirred in a heat bath at 110° C., allowing the MeOH and N,N-dimethylformamide dimethylacetal to boil off freely for 2 h. The reaction mixture was allowed to cool to ambient temperature and the resultant precipitate was filtered off, washed several times with Et₂O, and air dried to give the title compound (6.24 g, 96%) as a white solid. LCMS (ES⁻) m/z 268.0, 270.2 [M-1]. LCMS (ES⁺) m/z 270.0, 272.0 [M+H]⁺. ¹H NMR (400 MHz, DMSO-d₆) δ ppm 10.66 (s, 1H), 7.89 (s, 1H), 7.70-7.76 (m, 2H), 7.62-7.69 (m, 2H), 2.83 (s, 6H).

### b) 1,1-dimethylethyl (3S)-3-{[3-(4-bromophenyl)-4H-1,2,4-triazol-4-yl]methyl}-1-pyrrolidinecarboxylate

N′-[(4-bromophenyl)carbonyl]-N,N-dimethylhydrazonoformamide (0.674 g, 2.497 mmol) and 1,1-dimethylethyl (3S)-3-(aminomethyl)-1-pyrrolidinecarboxylate (0.500 g, 2.497 mmol) were combined in a sealed tube, purged with nitrogen, and heated at 110° C. for 16 h with stirring after which time a homogenous amber liquid was obtained. The reaction mixture was heated for an additional hour under house vacuum at which point no additional effervescence from the reaction mixture was noted and analysis by LCMS indicated complete conversion to the desired product, the hydrazide, and a third product of target mass. The reaction mixture was cooled to ambient temperature and triturated with 5 mL CH₂Cl₂ to give a fine precipitate which was filtered off to give 4-bromobenzohydrazide as a white solid. The filtrate was purified via flash chromatography (EtOAc, then 100% EtOAc to 10% MeOH/CH₂Cl₂) to give the title compound (0.532 g, 52%) as a white foam. LCMS (ES⁺) m/z 407.1, 409.1 [M+H]⁺. ¹H NMR (400 MHz, CDCl₃) δ ppm 8.24 (s, 1H), 7.68 (d, J=8.42 Hz, 2H), 7.48 (d, J=8.52 Hz, 2H), 3.96-4.14 (m, 2H), 3.18-3.53 (m, 3H), 2.86-3.06 (m, 1H), 2.40-2.57 (m, 1H), 1.83-2.00 (m, 1 H), 1.45-1.55 (m, 1H), 1.43 (s, 9H).

### c) 3-(4-bromophenyl)-4-{[(3S)-1-(cyclopropylcarbonyl)-3-pyrrolidinyl]methyl}-4H-1,2,4-triazole

1,1-dimethylethyl (3S)-3-{[3-(4-bromophenyl)-4H-1,2,4-triazol-4-yl]methyl}-1-pyrrolidinecarboxylate (0.33 g, 0.810 mmol) was treated with 4 N HCl in 1,4-dioxane (5 mL, 20.00 mmol) at 25° C. with stirring for 2 h. The reaction mixture was concentrated in vacuo, dissolved in CH₂Cl₂ and DIPEA (0.425 mL, 2.431 mmol), and treated with cyclopropanecarbonyl chloride (0.169 g, 1.620 mmol) at 25° C. The reaction mixture was washed with water, dried over MgSO₄, filtered, and concentrated in vacuo. The residue was purified by flash chromatography (EtOAc, then 100% EtOAc to 10% MeOH/CH₂Cl₂) to give the title compound as a white foam. LCMS (ES⁺) m/z 375.0, 377.0 [M+H]⁺. ¹H NMR (400 MHz, CDCl₃) δ ppm 8.28 (m, 1H), 7.66-7.72 (m, 2H), 7.47-7.54 (m, 2H), 3.97-4.21 (m, 2H), 3.36-3.73 (m, 3H), 3.08-3.26 (m, 1H), 2.43-2.69 (m, 1H), 1.92-2.10 (m, 1H), 1.38-1.68 (m, 2H), 0.98 (m, 2H), 0.72-0.81 (m, 2H).

### d) 6-[4-(4-{[(3S)-1-(cyclopropylcarbonyl)-3-pyrrolidinyl]methyl}-4H-1,2,4-triazol-3-yl)phenyl]-1H-indole

3-(4-bromophenyl)-4-{[(3S)-1-(cyclopropylcarbonyl)-3-pyrrolidinyl]methyl}-4H-1,2,4-triazole (0.093 g, 0.248 mmol), 1H-indol-6-ylboronic acid (0.080 g, 0.496 mmol), and Pd(PPh₃)₄ (0.014 g, 0.012 mmol) were combined in DME (4 mL) and 2 M aq. Na₂CO₃ (4.00 mL), purged with nitrogen, and irradiated in a microwave reactor for 45 min at 100° C. The reaction mixture was partitioned between EtOAc and water and the organic phase was isolated, dried over MgSO₄, filtered, and concentrated in vacuo. The residue was purified by flash chromatography (EtOAc, then 100% EtOAc to 10% MeOH/CH₂Cl₂) to afford the title compound (0.064 g, 63%) as a white foam. LCMS (ES⁺) m/z 412.02 [M+H]⁺. ¹H NMR (400 MHz, CD₃OD) δ ppm 8.72 (d, J=8.61 Hz, 1H), 7.89 (dd, J=8.22, 2.45 Hz, 2H), 7.68-7.77 (m, 3H), 7.65 (d, J=8.32 Hz, 1H), 7.38 (d, J=8.32 Hz, 1H), 7.31 (d, J=3.03 Hz, 1H), 6.48 (d, J=2.84 Hz, 1H), 4.19-4.36 (m, 2H), 3.00-3.73 (m, 4 H), 2.53-2.78 (m, 1H), 1.84-2.08 (m, 1H), 1.48-1.74 (m, 2H), 0.65-0.89 (m, 4H).

### Example 15

### 4-{[(3S)-1-(cyclopropylcarbonyl)-3-pyrrolidinyl]methyl}-3-(2′,4′-dichloro-4-biphenylyl)-5-methyl-4H-1,2,4-triazole

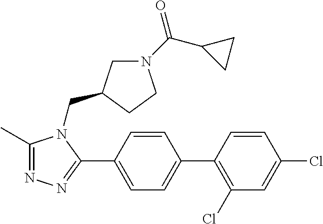 [SMILES: Cc1nnc(-c2ccc(-c3ccc(Cl)cc3Cl)cc2)n1C[C@@H]1CCN(C(=O)C2CC2)C1]

### a) (1E)-N′-[(4-bromophenyl)carbonyl]-N,N-dimethylethanehydrazonamide

4-bromobenzohydrazide (3.60 g, 16.74 mmol) and N,N-dimethyl-1,1-bis(methyloxy)ethanamine (10 mL, 75 mmol) were combined in a round bottom flask and stirred in a heat bath at 125° C. for 2 h after which time complete solution was obtained. The reaction mixture was allowed to cool to ambient temperature and the resultant precipitate was filtered off, washed several times with Et₂O, and air dried to give the title compound (1.12 g, 24%) as an ivory solid. LCMS (ES⁺) m/z 284.0, 286.0 [M+H]⁺. ¹H NMR (400 MHz, DMSO-d₆) δ ppm 10.30 (s, 1H), 7.75 (d, J=8.52 Hz, 2H), 7.62-7.67 (m, 1H), 2.92 (s, 6H), 1.86 (s, 3H).

### b) 1,1-dimethylethyl (3S)-3-{[3-(4-bromophenyl)-5-methyl-4H-1,2,4-triazol-4-yl]methyl}-1-pyrrolidinecarboxylate

N′-[(4-bromophenyl)carbonyl]-N,N-dimethylethanehydrazonamide (1.12 g, 3.94 mmol) and 1,1-dimethylethyl (3S)-3-(aminomethyl)-1-pyrrolidinecarboxylate (0.789 g, 3.94 mmol) were combined in CH₂Cl₂, concentrated in vacuo, and heated neat at 110° C. for 1 h. A small amount of MeOH was added to the reaction mixture to achieve homogeneity and was then allowed to boil off as the reaction mixture was continued to be heated at 110° C. for 16 h. The crude reaction mixture was purified by flash chromatography (0-10% MeOH/CH₂Cl₂). Fractions containing the desired product were combined and concentrated in vacuo to give impure product as a clear oil. The impure product was crystallized from Et₂O to afford the title compound (0.97 g, 58%) as a white solid. LCMS (ES⁺) m/z 420.9, 422.9 [M+H]⁺. ¹H NMR (400 MHz, DMSO-d₆) δ ppm 7.70-7.78 (m, 2H), 7.60 (d, J=8.49 Hz, 2H), 3.98-4.14 (m, 2H), 2.96-3.18 (m, 3H), 2.58-2.77 (m, 1H), 2.44 (s, 3H), 2.20-2.55 (m, 2H), 1.67 (dq, J=12.41, 6.21 Hz, 1H), 1.34 (s, 9H).

### c) 3-(4-bromophenyl)-4-{[(3S)-1-(cyclopropylcarbonyl)-3-pyrrolidinyl]methyl}-5-methyl-4H-1,2,4-triazole

1,1-dimethylethyl (3S)-3-{[3-(4-bromophenyl)-5-methyl-4H-1,2,4-triazol-4-yl]methyl}-1-pyrrolidinecarboxylate (0.97 g, 2.302 mmol) was treated with 4 N HCl in 1,4-dioxane (17 mL, 68.0 mmol) at 25° C. for 30 min which resulted in precipitation of a sticky white gum. The reaction mixture was concentrated in vacuo and pumped dry under high vacuum. The residue was dissolved in CH₂Cl₂ (20 mL) and DIPEA (1.206 mL, 6.91 mmol) and was treated with dropwise addition of cyclopropanecarbonyl chloride (0.289 g, 2.76 mmol) in CH₂Cl₂ (5 mL). The reaction mixture was stirred at 25° C. for 2 h after which time analysis by LCMS indicated complete reaction. The reaction mixture was washed with water, dried over MgSO₄, filtered, and concentrated in vacuo. The residue was purified by flash chromatography (0-10% MeOH/CH₂Cl₂) to yield the title compound (0.71 g, 79%) as a white foam. LCMS (ES⁺) m/z 388.9, 390.9 [M+H]⁺. ¹H NMR (400 MHz, DMSO-d₆) δ ppm 7.69-7.79 (m, 2H), 7.54-7.67 (m, 2H), 4.10 (dd, J=9.83, 7.90 Hz, 2H), 2.73-3.55 (m, 4H), 2.22-2.48 (m, 4H), 1.64-1.85 (m, 1H), 1.28-1.63 (m, 2 H), 0.59-0.70 (m, 4H).

### d) 4-{[(3S)-1-(cyclopropylcarbonyl)-3-pyrrolidinyl]methyl}-3-(2′,4′-dichloro-4-biphenylyl)-5-methyl-4H-1,2,4-triazole

3-(4-bromophenyl)-4-{[(3S)-1-(cyclopropylcarbonyl)-3-pyrrolidinyl]methyl}-5-methyl-4H-1,2,4-triazole (0.078 g, 0.200 mmol), (2,4-dichlorophenyl)boronic acid (0.076 g, 0.400 mmol), and Pd(PPh₃)₄ (0.012 g, 10.00 μmol) were combined in DME (4 mL) and 2 M aq. Na₂CO₃ (4.00 mL), purged with nitrogen, and irradiated in a microwave reactor for 1 h at 110° C. The organic phase was isolated, loaded onto silica, and purified via flash chromatography (100% EtOAc to 10% MeOH/CH₂Cl₂) to give the title compound (0.0817 g, 90%) as a white foam. LCMS (ES⁺) m/z 454.9, 456.9 [M+H]⁺. ¹H NMR (400 MHz, DMSO-d₆) δ ppm 7.49-7.80 (m, 7H), 4.09-4.22 (m, 2H), 2.82-3.59 (m, 4H), 2.46 (s, 3 H), 2.33-2.53 (m, 1H), 1.66-1.88 (m, 1H), 1.28-1.64 (m, 2H), 0.57-0.71 (m, 4H).

### Example 16

### 6-[4-(4-{[(3S)-1-(cyclopropylcarbonyl)-3-pyrrolidinyl]methyl}-5-methyl-4H-1,2,4-triazol-3-yl)phenyl]-1H-indole

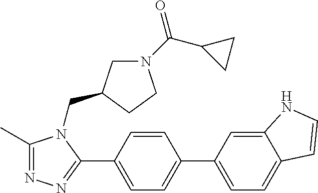 [SMILES: Cc1nnc(-c2ccc(-c3ccc4cc[nH]c4c3)cc2)n1C[C@@H]1CCN(C(=O)C2CC2)C1]

Following the procedure described in Example 15d with 1H-indol-6-ylboronic acid afforded the title compound (0.0677 g, 80%) as a white solid. LCMS (ES⁺) m/z 426.0 [M+H]⁺. ¹H NMR (400 MHz, DMSO-d₆) δ ppm 11.25 (br. s, 1H), 7.85 (m, 2H), 7.61-7.75 (m, 4H), 7.34-7.44 (m, 2H), 6.47 (br. s., 1H), 4.16 (dd, J=12.67, 7.59 Hz, 2H), 2.81-3.56 (m, 4H), 2.46 (s, 3H), 2.31-2.61 (m, 1H), 1.67-1.89 (m, 1H), 1.34-1.63 (m, 2H), 0.62 (m, 4H).

### Example 17

### 3-[4-(1-benzofuran-5-yl)phenyl]-4-{[(3S)-1-(cyclopropylcarbonyl)-3-pyrrolidinyl]methyl}-5-methyl-4H-1,2,4-triazole

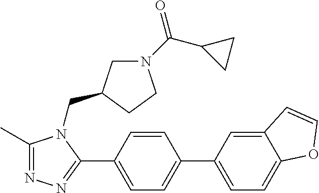 [SMILES: Cc1nnc(-c2ccc(-c3ccc4occc4c3)cc2)n1C[C@@H]1CCN(C(=O)C2CC2)C1]

Following the procedure described in Example 15d with 5-(4,4,5,5-tetramethyl-1,3,2-dioxaborolan-2-yl)-1-benzofuran afforded the title compound (0.0783 g, 92%) as a white foam. LCMS (ES⁺) m/z 427.0 [M+H]⁺. ¹H NMR (400 MHz, DMSO-d₆) δ ppm 8.06 (d, J=2.06 Hz, 1H), 8.03 (t, J=1.76 Hz, 1H), 7.87 (d, J=7.93 Hz, 2H), 7.67-7.78 (m, 4H), 7.04 (s, 1H), 4.16 (dd, J=11.16, 7.83 Hz, 2H), 2.79-3.57 (m, 4H), 2.46 (s, 3H), 2.33-2.52 (m, 1H), 1.67-1.87 (m, 1H), 1.32-1.62 (m, 2H), 0.57-0.67 (m, 4H).

### Example 18

### 4-{[(3S)-1-(cyclopropylcarbonyl)-3-pyrrolidinyl]methyl}-3-(2′,4′-dichloro-4-biphenylyl)-4H-1,2,4-triazole

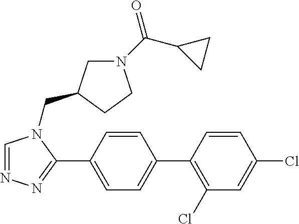 [SMILES: O=C(C1CC1)N1CC[C@@H](Cn2cnnc2-c2ccc(-c3ccc(Cl)cc3Cl)cc2)C1]

A mixture of 3-(4-bromophenyl)-4-{[(3S)-1-(cyclopropylcarbonyl)-3-pyrrolidinyl]methyl}-4H-1,2,4-triazole (0.213 mmol), (2,4-dichlorophenyl)boronic acid (0.213 mmol), and Pd(PPh₃)₄ (0.021 mmol) in CH₃CN (2 mL) and 0.5 M aq. Na₂CO₃ (2 mL) was purged with nitrogen and heated at 90° C. overnight. The reaction mixture was partitioned between EtOAc and water and the organic phase was isolated, washed with brine, dried over Na₂SO₄, filtered, and concentrated in vacuo. The residue was purified by reverse phase HPLC (30-60% CH₃CN/water with 0.1% TFA) to afford the trifluoroacetate salt of the title compound (0.036 g, 30%) as a colorless solid. LCMS (ES⁺) m/z 440.9, 443.9 [M+H]⁺.

### Example 19

### 5-[4-(4-{[(3S)-1-(cyclopropylcarbonyl)-3-pyrrolidinyl]methyl}-4H-1,2,4-triazol-3-yl)phenyl]-1H-indazole

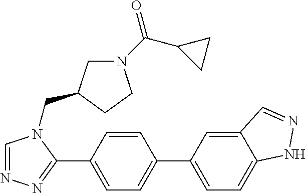 [SMILES: O=C(C1CC1)N1CC[C@@H](Cn2cnnc2-c2ccc(-c3ccc4[nH]ncc4c3)cc2)C1]

Following the procedure described in Example 18a with 1,1-dimethylethyl 5-(4,4,5,5-tetramethyl-1,3,2-dioxaborolan-2-yl)-1H-indazole-1-carboxylate afforded the trifluoroacetate salt of the title compound (0.030 g, 34%). LCMS (ES⁺) m/z 413.0 [M+H]⁺.

### Example 20

### 3-[2′-chloro-4′-(methyloxy)-4-biphenylyl]-4-{[(3S)-1-(cyclopropylcarbonyl)-3-pyrrolidinyl]methyl}-4H-1,2,4-triazole

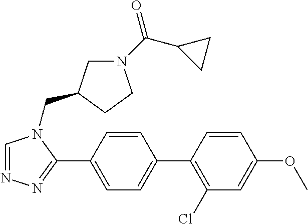 [SMILES: COc1ccc(-c2ccc(-c3nncn3C[C@@H]3CCN(C(=O)C4CC4)C3)cc2)c(Cl)c1]

Following the procedure described in Example 18a with [2-chloro-4-(methyloxy)phenyl]boronic acid afforded the trifluoroacetate salt of the title compound (0.052 g, 58%). LCMS (ES⁺) m/z 436.9, 438.9 [M+H]⁺.

### Example 21

### 5-[4-(1-{[(3S)-1-(cyclopropylcarbonyl)-3-pyrrolidinyl]methyl}-1H-imidazol-2-yl)phenyl]-1H-indole

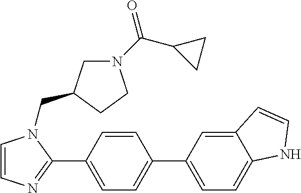 [SMILES: O=C(C1CC1)N1CC[C@@H](Cn2ccnc2-c2ccc(-c3ccc4[nH]ccc4c3)cc2)C1]

### a) 1,1-dimethylethyl 3-{[(methylsulfonyl)oxy]methyl}-1-pyrrolidinecarboxylate

A solution of 1,1-dimethylethyl 3-(hydroxymethyl)-1-pyrrolidinecarboxylate (3.051 mmol) and DIPEA (4.576 mmol) in CH₂Cl₂ (10 mL) was cooled to 0° C., treated with MsCl (3.051 mmol), and stirred at 0° C. for 2 h. The reaction mixture was then diluted with water and CH₂Cl₂ (50 mL). After separating the layers, the aqueous layer was further extracted with CH₂Cl₂ (2×20 mL). The combined organic layers were dried over Na₂SO₄ and concentrated in vacuo to provide the crude title compound (0.838 g) as an amber oil. LCMS (ES⁺) m/z 280 [M+H]⁺.

### b) 1,1-dimethylethyl (3S)-3-{[2-(4-bromophenyl)-1H-imidazol-1-yl]methyl}-1-pyrrolidinecarboxylate

A flask containing 2-(4-bromophenyl)-1H-imidazole (1.345 mmol) and NaH (60% dispersion in mineral oil, 1.614 mmol) under nitrogen was cooled to 0° C. and treated with dry THF (2 mL). After 20 min, a solution of 1,1-dimethylethyl 3-{[(methylsulfonyl)oxy]methyl}-1-pyrrolidinecarboxylate (1.345 mmol) in dry THF (2 mL) was added to the reaction mixture and the mixture was allowed to warm to room temperature. After stirring overnight at room temperature (minor product formed), the reaction mixture was heated at 80° C. overnight (˜70% conversion). The reaction mixture was then partitioned between EtOAc and dilute NaHCO₃ solution and the aqueous layer was further extracted twice with EtOAc. The combined organic layers were dried over Na₂SO₄ and were concentrated. Purification of the residue by flash chromatography (1-10% ethanol/EtOAc) and then repurification of impure product by flash chromatography (1% ethanol/EtOAc) gave the title compound (243 mg, 44%) as a colorless solid. LCMS (ES⁺) m/z 406, 408 [M+H]⁺.

### c) 2-(4-bromophenyl)-1-{[(3S)-1-(cyclopropylcarbonyl)-3-pyrrolidinyl]methyl}-1H-imidazole

A solution of 1,1-dimethylethyl 3-{[2-(4-bromophenyl)-1H-imidazol-1-yl]methyl}-1-pyrrolidinecarboxylate (0.591 mmol) in CH₂Cl₂ (2 mL) was treated with 4 M HCl in 1,4-dioxane (0.59 mL) and stirred at room temperature until analysis by HPLC indicated consumption of starting material with formation of a single product. The reaction mixture was concentrated in vacuo. The residue was dissolved in CH₂Cl₂ (10 mL) and treated with DIPEA (0.31 mL) and cyclopropanecarbonyl chloride (0.650 mmol). After analysis by HPLC indicated the reaction was complete, the mixture was diluted with CH₂Cl₂ and washed with saturated aq. NaHCO₃ (3×20 mL). The organic layer was dried over Na₂SO₄, filtered, and concentrated in vacuo to give the crude title compound (quantitative) as an ivory crystalline solid. LCMS (ES⁺) m/z 374, 376 [M+H]⁺.

### d) 5-[4-(1-{[(3S)-1-(cyclopropylcarbonyl)-3-pyrrolidinyl]methyl}-1H-imidazol-2-yl)phenyl]-1H-indole

A mixture of 2-(4-bromophenyl)-1-{[1-(cyclopropylcarbonyl)-3-pyrrolidinyl]methyl}-1H-imidazole (0.134 mmol), 1H-indol-5-ylboronic acid (0.150 mmol), NaHCO₃ (0.401 mmol), and Pd(PPh₃)₄ (0.013 mmol) in DMF (5 mL) and water (5 mL) was heated at 80° C. for 2 h. The reaction mixture was diluted with water and the organics were extracted with EtOAc. The organic phase was dried over Na₂SO₄, filtered, and concentrated in vacuo. Purification of the residue by reverse phase HPLC (CH₃CN/water with 0.1% TFA) followed by subsequent neutralization of the product fractions with addition of aq. NaHCO₃, extraction of the organics with CH₂Cl₂, drying over Na₂SO₄, and concentrating in vacuo afforded the title compound (0.043 g, 78%) as an amorphous solid. LCMS (ES⁺) m/z 411.2 [M+H]⁺.

### Example 22

### 6-[4-(1-{[(3S)-1-(cyclopropylcarbonyl)-3-pyrrolidinyl]methyl}-1H-imidazol-2-yl)phenyl]-1H-indole

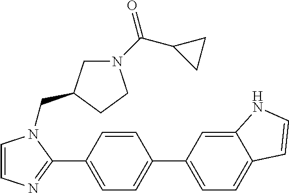 [SMILES: O=C(C1CC1)N1CC[C@@H](Cn2ccnc2-c2ccc(-c3ccc4cc[nH]c4c3)cc2)C1]

Following the procedure described in Example 21d with 1H-indol-6-ylboronic acid afforded the title compound (0.039 g, 71%) as an amorphous solid. LCMS (ES⁺) m/z 411.2 [M+H]⁺.

### Example 23

### 2-(3′-chloro-4-biphenylyl)-1-{[(3S)-1-(cyclopropylcarbonyl)-3-pyrrolidinyl]methyl}-1H-imidazole

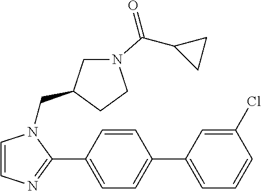 [SMILES: O=C(C1CC1)N1CC[C@@H](Cn2ccnc2-c2ccc(-c3cccc(Cl)c3)cc2)C1]

Following the procedure described in Example 21d with (3-chlorophenyl)boronic acid afforded the title compound (0.038 g, 70%) as an amorphous solid. LCMS (ES⁺) m/z 406.1 [M+H]⁺.

### Example 24

### 1-{[1-(cyclopropylcarbonyl)-3-pyrrolidinyl]methyl}-2-[4′-(methyloxy)-4-biphenylyl]-1H-imidazole

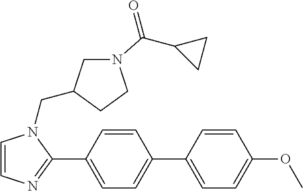 [SMILES: COc1ccc(-c2ccc(-c3nccn3CC3CCN(C(=O)C4CC4)C3)cc2)cc1]

Following the procedure described in Example 21d with 2-(4-bromophenyl)-1-{[1-(cyclopropylcarbonyl)-3-pyrrolidinyl]methyl}-1H-imidazole and [4-(methyloxy)phenyl]boronic acid afforded the title compound (0.035 g, 65%) as an amorphous solid. LCMS (ES⁺) m/z 402.2 [M+H]⁺.

### Example 25

### 1-{[1-(cyclopropylcarbonyl)-3-pyrrolidinyl]methyl}-2-(3′-fluoro-4′-methyl-4-biphenylyl)-1H-imidazole

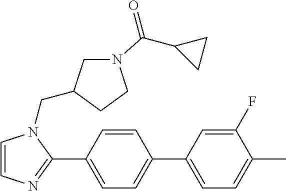 [SMILES: Cc1ccc(-c2ccc(-c3nccn3CC3CCN(C(=O)C4CC4)C3)cc2)cc1F]

Following the procedure described in Example 21d with 2-(4-bromophenyl)-1-{[1-(cyclopropylcarbonyl)-3-pyrrolidinyl]methyl}-1H-imidazole and (3-fluoro-4-methylphenyl)boronic acid afforded the title compound (0.041 g, 76%) as an amorphous solid. LCMS (ES⁺) m/z 404.2 [M+H]⁺.

### Example 26

### 2-(4-biphenylyl)-1-{[(3S)-1-(cyclopropylcarbonyl)-3-pyrrolidinyl]methyl}-1H-imidazole

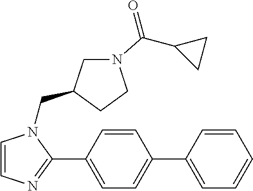 [SMILES: O=C(C1CC1)N1CC[C@@H](Cn2ccnc2-c2ccc(-c3ccccc3)cc2)C1]

### a) 1,1-dimethylethyl (3R)-3-{[(methylsulfonyl)oxy]methyl}-1-pyrrolidinecarboxylate

Following the procedure described in Example 21a with 1,1-dimethylethyl (3R)-3-(hydroxymethyl)-1-pyrrolidinecarboxylate afforded the title compound (2.28 g, 98%) as an amber resin.

### b) 1,1-dimethylethyl (3S)-3-{[2-(4-bromophenyl)-1H-imidazol-1-yl]methyl}-1-pyrrolidinecarboxylate

A flask containing 2-(4-bromophenyl)-1H-imidazole (1.793 mmol) and NaH (60% dispersion in mineral oil, 2.5 mmol) under nitrogen was cooled to 0° C. and treated with dry THF (8 mL). After 20 min at room temperature, a solution of 1,1-dimethylethyl (3R)-3-{[(methylsulfonyl)oxy]methyl}-1-pyrrolidinecarboxylate (1.793 mmol) in dry THF (4 mL) was added to the reaction mixture and the mixture was heated to 80° C. overnight (˜10% conversion by LCMS). Additional NaH (60% dispersion in mineral oil, 0.75 mmol) was added and the reaction mixture was continued to be heated at 80° C. until it had reached ˜50% conversion by LCMS. The reaction mixture was quenched with water and then partitioned between EtOAc and dilute NaHCO₃ solution. The aqueous layer was washed with EtOAc and the combined organic layers were dried over Na₂SO₄ and were concentrated. Purification of the residue by flash chromatography (1-3% ethanol/EtOAc) gave the title compound (319 mg, 44%). LCMS (ES⁺) m/z 406, 408 [M+H]⁺.

### c) 2-(4-bromophenyl)-1-{[(3S)-1-(cyclopropylcarbonyl)-3-pyrrolidinyl]methyl}-1H-imidazole

Following the procedure described in Example 21c with 1,1-dimethylethyl (3S)-3-{[2-(4-bromophenyl)-1H-imidazol-1-yl]methyl}-1-pyrrolidinecarboxylate afforded the title compound as a light amber resin. LCMS (ES⁺) m/z 374, 376 [M+H]⁺.

### d) 2-(4-biphenylyl)-1-{[(3S)-1-(cyclopropylcarbonyl)-3-pyrrolidinyl]methyl}-1H-imidazole

Following the procedure described in Example 21d with 2-(4-bromophenyl)-1-{[(3S)-1-(cyclopropylcarbonyl)-3-pyrrolidinyl]methyl}-1H-imidazole and phenylboronic acid and heating at 80° C. overnight afforded the title compound (0.033 g, 64%) as an amorphous solid. LCMS (ES⁺) m/z 372 [M+H]⁺.

### Example 27

### 5-[4-(1-{[1-(cyclopropylcarbonyl)-3-pyrrolidinyl]methyl}-1H-imidazol-2-yl)phenyl]-1H-indole

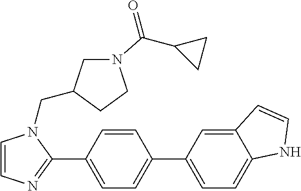 [SMILES: O=C(C1CC1)N1CCC(Cn2ccnc2-c2ccc(-c3ccc4[nH]ccc4c3)cc2)C1]

Following the procedure described in Example 21d with 2-(4-bromophenyl)-1-{[1-(cyclopropylcarbonyl)-3-pyrrolidinyl]methyl}-1H-imidazole and heating at 80° C. overnight afforded the title compound, which was isolated as a trifluoroacetate salt. LCMS (ES⁺) m/z 411 [M+H]⁺.

### Example 28

### 2-(3′-chloro-4-biphenylyl)-1-{[1-(cyclopropylcarbonyl)-3-pyrrolidinyl]methyl}-1H-imidazole

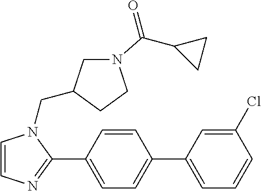 [SMILES: O=C(C1CC1)N1CCC(Cn2ccnc2-c2ccc(-c3cccc(Cl)c3)cc2)C1]

Following the procedure described in Example 23a with 2-(4-bromophenyl)-1-{[1-(cyclopropylcarbonyl)-3-pyrrolidinyl]methyl}-1H-imidazole and heating at 80° C. overnight afforded the title compound, which was isolated as a trifluoroacetate salt. LCMS (ES⁺) m/z 406 [M+H]⁺.

### Example 29

### 2-(4′-chloro-4-biphenylyl)-1-{[(3S)-1-(cyclopropylcarbonyl)-3-pyrrolidinyl]methyl}-1H-imidazole

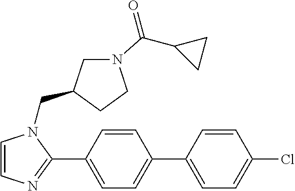 [SMILES: O=C(C1CC1)N1CC[C@@H](Cn2ccnc2-c2ccc(-c3ccc(Cl)cc3)cc2)C1]

Following the procedure described in Example 28a with (4-chlorophenyl)boronic acid afforded the title compound, which was isolated as a trifluoroacetate salt. LCMS (ES⁺) m/z 406 [M+H]⁺.

### Example 30

### 1-{[(3S)-1-(cyclopropylcarbonyl)-3-pyrrolidinyl]methyl}-2-(2′,4′-dichloro-4-biphenylyl)-1H-imidazole

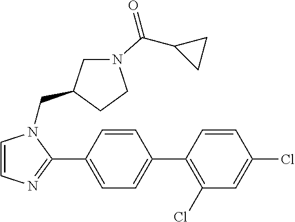 [SMILES: O=C(C1CC1)N1CC[C@@H](Cn2ccnc2-c2ccc(-c3ccc(Cl)cc3Cl)cc2)C1]

Following the procedure described in Example 26d with (2,4-dichlorophenyl)boronic acid afforded the title compound (58%). LCMS (ES⁺) m/z 440, 442 [M+H]⁺.

### Example 31

### 1-{[(3S)-1-(cyclopropylcarbonyl)-3-pyrrolidinyl]methyl}-2-(4′-fluoro-4-biphenylyl)-1H-imidazole

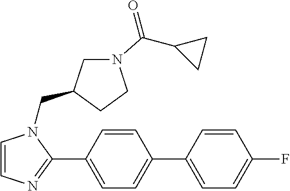 [SMILES: O=C(C1CC1)N1CC[C@@H](Cn2ccnc2-c2ccc(-c3ccc(F)cc3)cc2)C1]

Following the procedure described in Example 26d with (4-fluorophenyl)boronic acid afforded the title compound (80%). LCMS (ES⁺) m/z 390 [M+H]⁺.

### Example 32

### 3-[4-(1-{[(3S)-1-(cyclopropylcarbonyl)-3-pyrrolidinyl]methyl}-1H-imidazol-2-yl)phenyl]pyridine

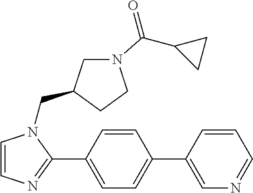 [SMILES: O=C(C1CC1)N1CC[C@@H](Cn2ccnc2-c2ccc(-c3cccnc3)cc2)C1]

Following the procedure described in Example 26d with 3-pyridinylboronic acid afforded the title compound (68%). LCMS (ES⁺) m/z 373 [M+H]⁺.

### Example 33

### 6-[4-(1-{[1-(cyclopropylcarbonyl)-3-pyrrolidinyl]methyl}-1H-imidazol-2-yl)phenyl]-1H-indole

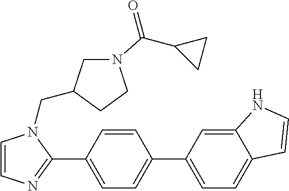 [SMILES: O=C(C1CC1)N1CCC(Cn2ccnc2-c2ccc(-c3ccc4cc[nH]c4c3)cc2)C1]

Following the procedure described in Example 26d with 2-(4-bromophenyl)-1-{[1-(cyclopropylcarbonyl)-3-pyrrolidinyl]methyl}-1H-imidazole and 1H-indol-6-ylboronic acid (2.0 eq) afforded the title compound (41%). LCMS (ES⁺) m/z 411.2 [M+H]⁺.

### Example 34

### 2-[4-(1-benzofuran-5-yl)phenyl]-1-{[(3S)-1-(cyclopropylcarbonyl)-3-pyrrolidinyl]methyl}-1H-imidazole

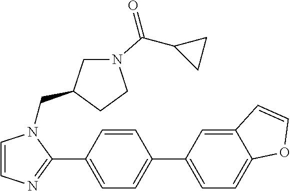 [SMILES: O=C(C1CC1)N1CC[C@@H](Cn2ccnc2-c2ccc(-c3ccc4occc4c3)cc2)C1]

Following the procedure described in Example 26d with 5-(4,4,5,5-tetramethyl-1,3,2-dioxaborolan-2-yl)-1-benzofuran afforded the title compound, which was isolated as a trifluoroacetate salt (49%). LCMS (ES⁺) m/z 412.3 [M+H]⁺.

### Example 35

### 1-{[1-(cyclopropylcarbonyl)-3-pyrrolidinyl]methyl}-4,5-dimethyl-2-[4′-(methyloxy)-4-biphenylyl]-1H-imidazole

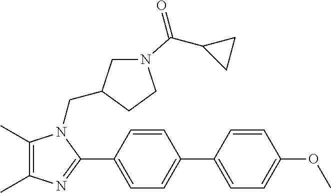

### a) 2-(4-bromophenyl)-4,5-dimethyl-1H-imidazole

To a slurry of ammonium acetate (2.9 mmol) in acetic acid (75 mL) was added 2,3-butanedione (0.29 mmol) followed by 4-bromobenzaldehyde (0.58 mmol). The mixture was heated at 60° C. overnight. The reaction was carefully poured into ammonium hydroxide (500 mL) and water (500 mL) and was then filtered. The solid was dissolved in EtOAc and washed with water and brine. The organic layer was dried over magnesium sulfate, filtered, and concentrated in vacuo. Purification of the residue by flash chromatography (50% EtOAc/petroleum ether) gave the title compound (13%).

### b) 1,1-dimethylethyl 3-{[2-(4-bromophenyl)-4,5-dimethyl-1H-imidazol-1-yl]methyl}-1-pyrrolidinecarboxylate

To a solution of 2-(4-bromophenyl)-4,5-dimethyl-1H-imidazole (1.2 mmol) in DMF (10 mL) was added K₂CO₃ (2.4 mmol) and then 1,1-dimethylethyl 3-{[(methylsulfonyl)oxy]methyl}-1-pyrrolidinecarboxylate (1.2 mmol) in CH₃CN (10 mL). The reaction mixture was stirred at 80° C. for overnight. Water was added and the organics were extracted with EtOAc (3×). The combined organic layers were washed with brine, dried over magnesium sulfate, filtered, and concentrated in vacuo. Purification of the residue by flash chromatography afforded the title compound.

### c) 2-(4-bromophenyl)-1-{[1-(cyclopropylcarbonyl)-3-pyrrolidinyl]methyl}-4,5-dimethyl-1H-imidazole

To a solution of 1,1-dimethylethyl 3-{[2-(4-bromophenyl)-4,5-dimethyl-1H-imidazol-1-yl]methyl}-1-pyrrolidinecarboxylate (1.15 mmol) in MeOH was added HCl in MeOH (5 M, 5 mL). After 2 h, the reaction was concentrated in vacuo and then taken up in CH₂Cl₂ (5 mL). DIPEA (1.38 mmol) and then cyclopropanecarbonyl chloride (1.3 mmol) were each added carefully and the reaction mixture was stirred at room temperature overnight. Water was added, and the organics were extracted with EtOAc. The organic layers were concentrated in vacuo and the residue was purified by flash chromatography to provide the title compound.

### d) 1-{[1-(cyclopropylcarbonyl)-3-pyrrolidinyl]methyl}-4,5-dimethyl-2-[4′-(methyloxy)-4-biphenylyl]-1H-imidazole

A mixture of 2-(4-bromophenyl)-1-{[1-(cyclopropylcarbonyl)-3-pyrrolidinyl]methyl}-4,5-dimethyl-1H-imidazole (0.37 mmol), [4-(methyloxy)phenyl]boronic acid (0.41 mmol), and Pd(PPh₃)₄ (10 mg) in 1,4-dioxane (2 mL) and 1 M aq. Na₂CO₃ (0.56 mL) was heated at 80° C. overnight. The reaction mixture was concentrated in vacuo and the resulting residue was purified by reverse phase HPLC to afford the title compound (50%) as a solid. LCMS (ES⁺) m/z 430.4 [M+H]⁺.

### Example 36

### 1-{[1-(cyclopropylcarbonyl)-3-pyrrolidinyl]methyl}-2-(2′,4′-dichloro-4-biphenylyl)-4,5-dimethyl-1H-imidazole

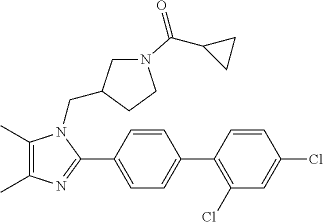 [SMILES: Cc1nc(-c2ccc(-c3ccc(Cl)cc3Cl)cc2)n(CC2CCN(C(=O)C3CC3)C2)c1C]

Following the procedure described in Example 35d with (2,4-dichlorophenyl)boronic acid gave the title compound (20%) as a solid. LCMS (ES⁺) m/z 468.3 [M+H]⁺.

### Example 37

### 2-[2′-chloro-4′-(methyloxy)-4-biphenylyl]-1-{[1-(cyclopropylcarbonyl)-3-pyrrolidinyl]methyl}-4,5-dimethyl-1H-imidazole

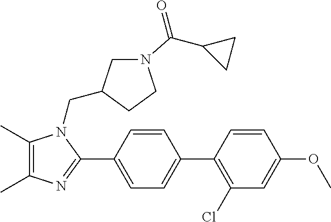

Following the procedure described in Example 35d with [2-chloro-4-(methyloxy)phenyl]boronic acid gave the title compound (27%) as a solid. LCMS (ES⁺) m/z 464.3 [M+H]⁺.

### Example 38

### 2-(3′-chloro-4′-fluoro-4-biphenylyl)-1-{[1-(cyclopropylcarbonyl)-3-pyrrolidinyl]methyl}-4,5-dimethyl-1H-imidazole

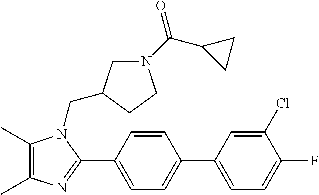 [SMILES: Cc1nc(-c2ccc(-c3ccc(F)c(Cl)c3)cc2)n(CC2CCN(C(=O)C3CC3)C2)c1C]

Following the procedure described in Example 35d with (3-chloro-4-fluorophenyl)boronic acid gave the title compound (48%) as a solid. LCMS (ES⁺) m/z 452.3 [M+H]⁺.

### Example 39

### 1-{[1-(cyclopropylcarbonyl)-3-pyrrolidinyl]methyl}-2-(4′-fluoro-3′-methyl-4-biphenylyl)-4,5-dimethyl-1H-imidazole

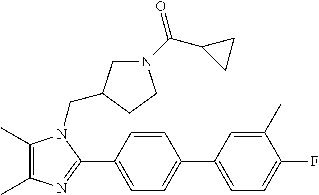 [SMILES: Cc1cc(-c2ccc(-c3nc(C)c(C)n3CC3CCN(C(=O)C4CC4)C3)cc2)ccc1F]

Following the procedure described in Example 35d with (4-fluoro-3-methylphenyl)boronic acid gave the title compound (58%) as a solid. LCMS (ES⁺) m/z 432.4 [M+H]⁺.

### Example 40

### 1-{[1-(cyclopropylcarbonyl)-3-pyrrolidinyl]methyl}-4,5-dimethyl-2-(4′-methyl-4-biphenylyl)-1H-imidazole

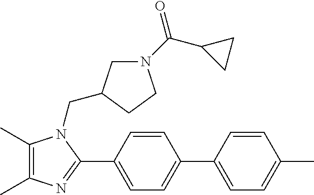

A mixture of 2-(4-bromophenyl)-1-{[1-(cyclopropylcarbonyl)-3-pyrrolidinyl]methyl}-4,5-dimethyl-1H-imidazole (0.50 mmol), (4-methylphenyl)boronic acid (0.60 mmol), and Pd(PPh₃)₄ (0.050 mmol) in 1,4-dioxane (5 mL) and 1 M aq. Na₂CO₃ (0.60 mL) was heated at 80° C. overnight. The reaction mixture was concentrated in vacuo and the resulting residue was purified by flash chromatography (33% EtOAc/petroleum ether) to afford the title compound (45%) as a solid. LCMS (ES⁺) m/z 414.2 [M+H]⁺.

### Example 41

### 1-{[1-(cyclopropylcarbonyl)-3-pyrrolidinyl]methyl}-2-(4′-fluoro-4-biphenylyl)-4,5-dimethyl-1H-imidazole

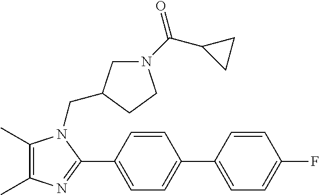 [SMILES: Cc1nc(-c2ccc(-c3ccc(F)cc3)cc2)n(CC2CCN(C(=O)C3CC3)C2)c1C]

A mixture of 2-(4-bromophenyl)-1-{[1-(cyclopropylcarbonyl)-3-pyrrolidinyl]methyl}-4,5-dimethyl-1H-imidazole (0.75 mmol), (4-fluorophenyl)boronic acid (0.75 mmol), Na₂CO₃ (28.71 mmol), and Pd(PPh₃)₄ (0.0075 mmol) in 1,4-dioxane (6 mL) and water (3 mL) was heated at 80° C. overnight. The reaction mixture was filtered and concentrated in vacuo. Purification of the residue by reverse phase HPLC and then preparative TLC afforded the title compound (10%) as a solid. LCMS (ES⁺) m/z 418.4 [M+H]⁺.

### Example 42

### 4′-(1-{[1-(cyclopropylcarbonyl)-3-pyrrolidinyl]methyl}-4,5-dimethyl-1H-imidazol-2-yl)-3-biphenylol

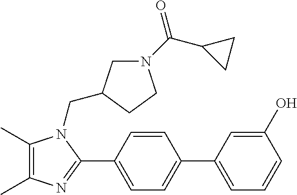 [SMILES: Cc1nc(-c2ccc(-c3cccc(O)c3)cc2)n(CC2CCN(C(=O)C3CC3)C2)c1C]

A mixture of 2-(4-bromophenyl)-1-{[1-(cyclopropylcarbonyl)-3-pyrrolidinyl]methyl}-4,5-dimethyl-1H-imidazole (0.75 mmol), (3-hydroxyphenyl)boronic acid (0.75 mmol), Na₂CO₃ (2.25 mmol), and Pd(PPh₃)₄ (0.075 mmol) in 1,4-dioxane (3 mL) and water (1 mL) was heated at 80° C. overnight. The reaction mixture was filtered and concentrated in vacuo. Purification of the residue by reverse phase HPLC and then preparative TLC afforded the title compound (16%) as a solid. LCMS (ES⁺) m/z 416.4 [M+H]⁺.

### Example 43

### 1-{[1-(cyclopropylcarbonyl)-3-pyrrolidinyl]methyl}-4,5-dimethyl-2-(3′-methyl-4-biphenylyl)-1H-imidazole

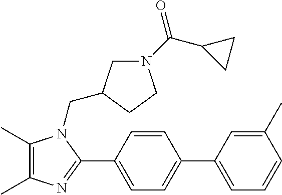

Following the procedure described in Example 42a with (3-methylphenyl)boronic acid gave the title compound (12%) as a solid. LCMS (ES⁺) m/z 414.4 [M+H]⁺.

### Example 44

### 2-(3′-chloro-4-biphenylyl)-1-{[1-(cyclopropylcarbonyl)-3-pyrrolidinyl]methyl}-4,5-dimethyl-1H-imidazole

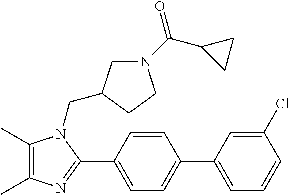 [SMILES: Cc1nc(-c2ccc(-c3cccc(Cl)c3)cc2)n(CC2CCN(C(=O)C3CC3)C2)c1C]

Following the procedure described in Example 42a with (3-chlorophenyl)boronic acid gave the title compound (23%) as a solid. LCMS (ES⁺) m/z 434.3 [M+H]⁺.

### Example 45

### 2-(4′-chloro-4-biphenylyl)-1-{[1-(cyclopropylcarbonyl)-3-pyrrolidinyl]methyl}-4,5-dimethyl-1H-imidazole

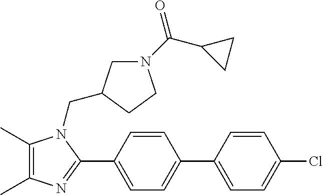 [SMILES: Cc1nc(-c2ccc(-c3ccc(Cl)cc3)cc2)n(CC2CCN(C(=O)C3CC3)C2)c1C]

Following the procedure described in Example 42a with (4-chlorophenyl)boronic acid gave the title compound (8%) as a solid. LCMS (ES⁺) m/z 434.3 [M+H]⁺.

### Example 46

### 1-{[1-(cyclopropylcarbonyl)-3-pyrrolidinyl]methyl}-5-methyl-2-(4′-methyl-4-biphenylyl)-1H-imidazole

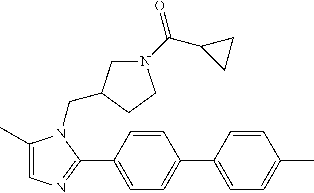

### a) 2-(4-bromophenyl)-4-methyl-1H-imidazole

A solution of pyruvaldehyde (40 wt. % in water, 108 mL) was added dropwise to a solution of 4-bromobenzaldehyde (108 mmol) in MeOH (300 mL) and 30% aq. ammonia (220 mL). The reaction mixture was heated at reflux for overnight. The reaction was concentrated, extracted with CH₂Cl₂, and concentrated in vacuo. Purification of the residue by flash chromatography gave the title compound (53%).

### b) 1,1-dimethylethyl 3-{[2-(4-bromophenyl)-5-methyl-1H-imidazol-1-yl]methyl}-1-pyrrolidinecarboxylate

To a solution of 2-(4-bromophenyl)-4-methyl-1H-imidazole (35 mmol) and 1,1-dimethylethyl 3-{[(methylsulfonyl)oxy]methyl}-1-pyrrolidinecarboxylate (35 mmol) in dry THF (100 mL) was added NaH (60% dispersion in mineral oil, 70 mmol). The reaction mixture was heated at reflux overnight. The reaction mixture was then carefully quenched with water and the organics were extracted with EtOAc. The combined organic layers were washed with brine, dried over magnesium sulfate, filtered, and concentrated in vacuo. Purification of the residue by flash chromatography afforded the title compound (57%).

### c) 2-(4-bromophenyl)-1-{[1-(cyclopropylcarbonyl)-3-pyrrolidinyl]methyl}-5-methyl-1H-imidazole

A solution of 1,1-dimethylethyl 3-{[2-(4-bromophenyl)-5-methyl-1H-imidazol-1-yl]methyl}-1-pyrrolidinecarboxylate (20 mmol) in 5 M HCl in MeOH (80 mL) was stirred overnight and then concentrated in vacuo. The residue was diluted with dry CH₂Cl₂ (80 mL), and then DIPEA (40 mmol) was added and the mixture was cooled. Cyclopropanecarbonyl chloride (22 mmol) was added dropwise and the reaction mixture was stirred at room temperature. Once complete, the reaction mixture was quenched with water and the organic layer was washed with NH₄Cl solution and brine. The aqueous layers were then extracted with CH₂Cl₂. The combined organic layers were dried over magnesium sulfate and were concentrated in vacuo. Purification of the residue by flash chromatography (1:10:20 MeOH:EtOAc:petroleum ether) gave the title compound (90%).

### d) 1-{[1-(cyclopropylcarbonyl)-3-pyrrolidinyl]methyl}-5-methyl-2-(4′-methyl-4-biphenylyl)-1H-imidazole

A mixture of 2-(4-bromophenyl)-1-{[1-(cyclopropylcarbonyl)-3-pyrrolidinyl]methyl}-5-methyl-1H-imidazole (0.44 mmol), (4-methylphenyl)boronic acid (0.53 mmol), K₂CO₃ (1.32 mmol), and Pd(PPh₃)₄ (30 mg) in 1,4-dioxane (2 mL) and water (0.5 mL) was heated at 100° C. overnight. The reaction mixture was concentrated in vacuo and the resulting residue was purified by preparative TLC to afford the title compound (34%) as a solid. LCMS (ES⁺) m/z 400.2 [M+H]⁺.

### Example 47

### 1-{[1-(cyclopropylcarbonyl)-3-pyrrolidinyl]methyl}-2-[4′-(ethyloxy)-4-biphenylyl]-5-methyl-1H-imidazole

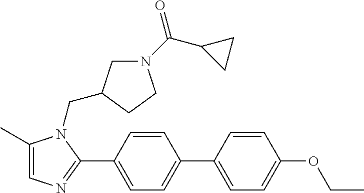 [SMILES: CCOc1ccc(-c2ccc(-c3ncc(C)n3CC3CCN(C(=O)C4CC4)C3)cc2)cc1]

Following the procedure described in Example 46d with [4-(ethyloxy)phenyl]boronic acid gave the title compound (32%) as a solid. LCMS (ES⁺) m/z 430.2 [M+H]⁺.

### Example 48

### 1-{[1-(cyclopropylcarbonyl)-3-pyrrolidinyl]methyl}-5-methyl-2-[4′-(methyloxy)-4-biphenylyl]-1H-imidazole

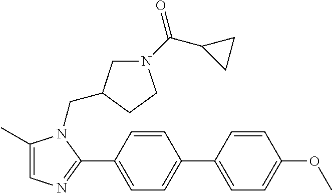

Following the procedure described in Example 46d with [4-(methyloxy)phenyl]boronic acid gave the title compound (33%) as a solid. LCMS (ES⁺) m/z 416.2 [M+H]⁺.

### Example 49

### 2-(4′-chloro-4-biphenylyl)-1-{[1-(cyclopropylcarbonyl)-3-pyrrolidinyl]methyl}-5-methyl-1H-imidazole

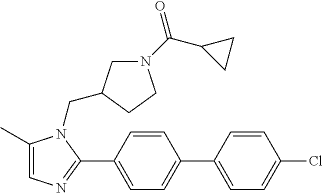 [SMILES: Cc1cnc(-c2ccc(-c3ccc(Cl)cc3)cc2)n1CC1CCN(C(=O)C2CC2)C1]

A mixture of 2-(4-bromophenyl)-1-{[1-(cyclopropylcarbonyl)-3-pyrrolidinyl]methyl}-5-methyl-1H-imidazole (0.51 mmol), (4-chlorophenyl)boronic acid (0.62 mmol), K₂CO₃ (1.8 mmol), and Pd(PPh₃)₄ (30 mg) in 1,4-dioxane (5 mL) and water (1 mL) was heated at 100° C. overnight. The reaction mixture was filtered and concentrated in vacuo, and the resulting residue was purified by reverse phase HPLC to afford the title compound as a solid. LCMS (ES⁺) m/z 420.2 [M+H]⁺.

### Example 50

### 1-{[1-(cyclopropylcarbonyl)-3-pyrrolidinyl]methyl}-5-methyl-2-(3′-methyl-4-biphenylyl)-1H-imidazole

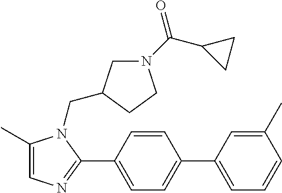

Following the procedure described in Example 49a with (3-methylphenyl)boronic acid gave the title compound as a solid. LCMS (ES⁺) m/z 400.2 [M+H]⁺.

### Example 51

### 2-(3′-chloro-4-biphenylyl)-1-{[1-(cyclopropylcarbonyl)-3-pyrrolidinyl]methyl}-5-methyl-1H-imidazole

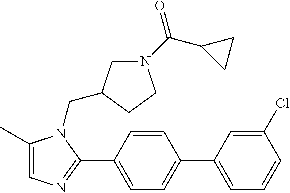 [SMILES: Cc1cnc(-c2ccc(-c3cccc(Cl)c3)cc2)n1CC1CCN(C(=O)C2CC2)C1]

Following the procedure described in Example 49a with (3-chlorophenyl)boronic acid gave the title compound as a solid. LCMS (ES⁺) m/z 420.2 [M+H]⁺.

### Example 52

### 1-{[1-(cyclopropylcarbonyl)-3-pyrrolidinyl]methyl}-2-(4′-fluoro-4-biphenylyl)-5-methyl-1H-imidazole

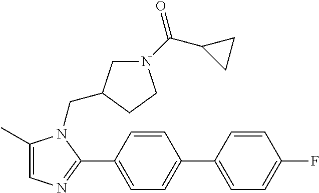 [SMILES: Cc1cnc(-c2ccc(-c3ccc(F)cc3)cc2)n1CC1CCN(C(=O)C2CC2)C1]

A mixture of 2-(4-bromophenyl)-1-{[1-(cyclopropylcarbonyl)-3-pyrrolidinyl]methyl}-5-methyl-1H-imidazole (0.160 mmol), (4-fluorophenyl)boronic acid (0.186 mmol), potassium phosphate tribasic trihydrate (0.240 mmol), and Pd(PPh₃)₄ (0.0016 mmol) in DMF (2 mL) and water (1 mL) was heated at 80° C. overnight. Water and EtOAc were added to the reaction mixture, the layers were separated, and the organic layer was washed with water and then concentrated. Purification of the residue by flash chromatography (1:4:8 MeOH:petroleum ether:EtOAc) afforded the title compound (37%) as a solid. LCMS (ES⁺) m/z 404.2 [M+H]⁺.

### Example 53

### 1-{[1-(cyclopropylcarbonyl)-3-pyrrolidinyl]methyl}-2-(2′,4′-dimethyl-4-biphenylyl)-5-methyl-1H-imidazole

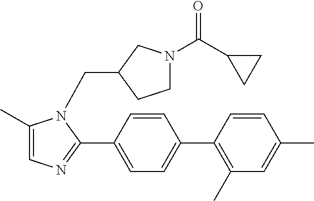

Following the procedure described in Example 52a with (2,4-dimethylphenyl)boronic acid at 70° C. gave the title compound (32%) as a solid. LCMS (ES⁺) m/z 414.2 [M+H]⁺.

### Example 54

### 1-{[1-(cyclopropylcarbonyl)-3-pyrrolidinyl]methyl}-2-(2′,4′-dichloro-4-biphenylyl)-5-methyl-1H-imidazole

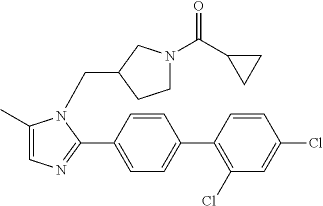 [SMILES: Cc1cnc(-c2ccc(-c3ccc(Cl)cc3Cl)cc2)n1CC1CCN(C(=O)C2CC2)C1]

Following the procedure described in Example 52a with (2,4-dichlorophenyl)boronic acid gave the title compound (27%) as a solid. LCMS (ES⁺) m/z 454.1 [M+H]⁺.

### Example 55

### 1-{[1-(cyclopropylcarbonyl)-3-pyrrolidinyl]methyl}-2-(4′-fluoro-3′-methyl-4-biphenylyl)-5-methyl-1H-imidazole

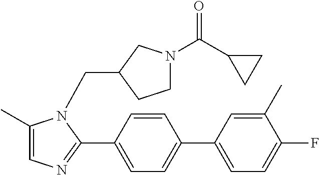 [SMILES: Cc1cc(-c2ccc(-c3ncc(C)n3CC3CCN(C(=O)C4CC4)C3)cc2)ccc1F]

Following the procedure described in Example 52a with (4-fluoro-3-methylphenyl)boronic acid gave the title compound (39%) as a solid. LCMS (ES⁺) m/z 418.2 [M+H]⁺.

### Example 56

### 2-[2′-chloro-4′-(methyloxy)-4-biphenylyl]-1-{[1-(cyclopropylcarbonyl)-3-pyrrolidinyl]methyl}-5-methyl-1H-imidazole

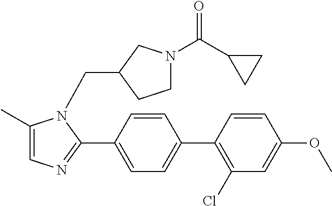

Following the procedure described in Example 52a with [2-chloro-4-(methyloxy)phenyl]boronic acid at 70° C. gave the title compound (41%) as a solid. LCMS (ES⁺) m/z 450.2 [M+H]⁺.

### Example 57

### 2-(3′-chloro-4′-fluoro-4-biphenylyl)-1-{[1-(cyclopropylcarbonyl)-3-pyrrolidinyl]methyl}-5-methyl-1H-imidazole

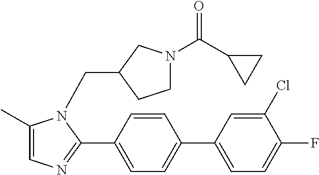 [SMILES: Cc1cnc(-c2ccc(-c3ccc(F)c(Cl)c3)cc2)n1CC1CCN(C(=O)C2CC2)C1]

A mixture of 2-(4-bromophenyl)-1-{[1-(cyclopropylcarbonyl)-3-pyrrolidinyl]methyl}-5-methyl-1H-imidazole (0.130 mmol), (3-chloro-4-fluorophenyl)boronic acid (0.155 mmol), K₂CO₃ (0.195 mmol), and Pd(PPh₃)₄ (0.0013 mmol) in 1,4-dioxane (1 mL) and water (0.3 mL) was heated at 80° C. overnight. The reaction mixture was concentrated and then water and EtOAc were added. The layers were separated and the organic layer was washed with water, dried, and then concentrated. Purification of the residue by flash chromatography (2:4:8 MeOH:petroleum ether:EtOAc) afforded the title compound (28%) as a solid. LCMS (ES⁺) m/z 438.2 [M+H]⁺.

### Example 58

### 3-[4-(1-benzofuran-5-yl)phenyl]-4-{[(3S)-1-(cyclopropylcarbonyl)-3-pyrrolidinyl]methyl}-4H-1,2,4-triazole

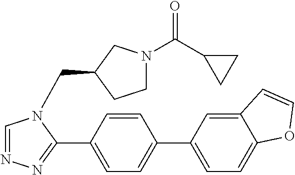 [SMILES: O=C(C1CC1)N1CC[C@@H](Cn2cnnc2-c2ccc(-c3ccc4occc4c3)cc2)C1]

### a) 1,1-dimethylethyl (3S)-3-[(methylsulfonyl)oxy]-1-pyrrolidinecarboxylate

To a 100 mL round bottom flask containing 1,1-dimethylethyl (3S)-3-hydroxy-1-pyrrolidinecarboxylate (20 g, 107 mmol) was added CH₂Cl₂ (300 mL) and Et₃N (24 mL, 172 mmol). The solution was cooled to 0° C. and MsCl (10 mL, 128 mmol) was added dropwise to the reaction. The reaction was stirred under nitrogen while the cooling bath was allowed to warm slowly to room temperature. After 3 h, analysis by LCMS indicated the reaction had progressed to completion. The mixture was diluted with CH₂Cl₂ (300 mL) and washed with 0.1 N aq. HCl and then with brine. The organic layer was dried over Na₂SO₄, filtered, and concentrated in vacuo. Purification of the crude oil by flash chromatography (0-5% MeOH/CH₂Cl₂) afforded the title compound (24 g, 85%). MS(ES)⁺ m/e 266.0 [M+H]⁺.

### b) 1,1-dimethylethyl (3R)-3-cyano-1-pyrrolidinecarboxylate

To a solution of 1,1-dimethylethyl (3S)-3-[(methylsulfonyl)oxy]-1-pyrrolidinecarboxylate (24 g, 90 mmol) in DMF (150 mL) was added NaCN (13.30 g, 271 mmol). The reaction mixture was stirred at 100° C. under nitrogen overnight, at which point analysis by TLC and NMR indicated the reaction was complete. The mixture was diluted with Et₂O (600 mL), stirred briefly, and filtered. The Et₂O filtrate was washed with 1:1 water:brine (7×), and the organic layer was dried over Na₂SO₄, filtered, and concentrated in vacuo. Purification of the residue by flash chromatography (5-50% EtOAc/hexanes) provided the title compound (6.4 g, 36%). MS(ES)⁺ m/e 266.0 [M+H]⁺.

### c) 1,1-dimethylethyl (3S)-3-(aminomethyl)-1-pyrrolidinecarboxylate

To a Parr hydrogenation vessel under nitrogen was added Raney Nickel catalyst (2 g, 22.42 mmol) followed by a solution of 1,1-dimethylethyl (3R)-3-cyano-1-pyrrolidinecarboxylate (6.0 g, 30.6 mmol) in ethanol (25 mL) and then Et₃N (2.5 mL, 17.94 mmol). The vessel was put under nitrogen, placed on a Parr hydrogenation shaker, and hydrogenated under 60 psi of hydrogen at room temperature overnight (pressure kept above 50 psi). After evacuation of hydrogen, the reaction mixture was filtered through Celite, washed with ethanol (50 mL), and concentrated in vacuo to afford the crude title compound (5.6 g, 70%). MS(ES)⁺ m/e 197.1 [M+H]⁺.

### d) N′-[(4-bromophenyl)carbonyl]-N,N-dimethylhydrazonoformamide

4-Bromobenzoichydrazide (10 g, 46.5 mmol) and N,N-dimethylformamide dimethylacetal (70 mL, 523 mmol) were stirred at 110° C. for 3 h as MeOH was freely allowed to boil off under a nitrogen atmosphere. The reaction was cooled to room temperature, and analysis by LCMS indicated the reaction had gone to completion. The mixture was filtered and the solid was washed with Et₂O (500 mL) and dried via air suction to provide the title compound (11.65 g, 93%). MS(ES)⁺ m/e 270.2, 272.3 [M+H]⁺.

### e) 1,1-dimethylethyl (3S)-3-{[3-(4-bromophenyl)-4H-1,2,4-triazol-4-yl]methyl}-1-pyrrolidinecarboxylate

A mixture of 1,1-dimethylethyl (3S)-3-(aminomethyl)-1-pyrrolidinecarboxylate (0.9 g, 4.49 mmol) and N′-[(4-bromophenyl)carbonyl]-N,N-dimethylhydrazonoformamide (1.214 g, 4.49 mmol) was heated at 110° C. overnight. The reaction was cooled and diluted with CH₂Cl₂ (6 mL), at which point a solid precipitated. The solid was filtered and washed with CH₂Cl₂. The filtrate was purified by flash chromatography (100% EtOAc to 10% MeOH/CH₂Cl₂) to afford the title compound. This process was repeated three more times and the products were combined to give the title compound (3.6 g, 49% overall yield). MS(ES)⁺ m/e 407.3, 409.3 [M+H]⁺.

### f) 3-(4-bromophenyl)-4-{[(3S)-1-(cyclopropylcarbonyl)-3-pyrrolidinyl]methyl}-4H-1,2,4-triazole

A solution of 1,1-dimethylethyl (3S)-3-{[3-(4-bromophenyl)-4H-1,2,4-triazol-4-yl]methyl}-1-pyrrolidinecarboxylate (3.6 g, 8.84 mmol) in 4 N HCl in 1,4-dioxane (35 mL, 140 mmol) was stirred for 1 h at room temperature. Analysis by LCMS indicated complete removal of the protecting group. The reaction was concentrated to dryness and allowed to dry under vacuum overnight. The residue was taken up in CH₂Cl₂ (40 mL) and cooled to 0° C. Diiospropylethylamine (4.7 mL, 26.9 mmol) was added, and 5 min later cyclopropanecarbonyl chloride (0.809 mL, 8.84 mmol) was added dropwise. After 30 min at 0° C., analysis by LCMS analysis revealed the reaction had proceeded to completion. The reaction was diluted with CH₂Cl₂ (20 mL) and washed with water. The organic layer was separated, dried over Na₂SO₄, filtered, and concentrated in vacuo. Purification of the residue by flash chromatography (0-10% MeOH/CH₂Cl₂) and then chiral SFC (Chiralpak AS-H, 50% MeOH/liquid carbon dioxide) afforded the title compound in >98% ee as a white glassy solid (2.25 g, 66%). MS(ES)⁺ m/e 375.0, 376.9 [M+H]⁺.

### g) 3-[4-(1-benzofuran-5-yl)phenyl]-4-{[(3S)-1-(cyclopropylcarbonyl)-3-pyrrolidinyl]methyl}-4H-1,2,4-triazole

A mixture of 3-(4-bromophenyl)-4-{[(3S)-1-(cyclopropylcarbonyl)-3-pyrrolidinyl]methyl}-4H-1,2,4-triazole (100 mg, 0.266 mmol), 5-(4,4,5,5-tetramethyl-1,3,2-dioxaborolan-2-yl)-1-benzofuran (65 mg, 0.266 mmol), and PdCl₂(dppf) (22 mg, 0.027 mmol) in 1,4-dioxane (2 mL) and 2 M aq. K₂CO₃ (1 mL) was heated at 100° C. for 1 h. The reaction was cooled to room temperature and allowed to stand such that the 1,4-dioxane layer separated from the aqueous layer. The 1,4-dioxane layer was removed, filtered through a plug of Celite® and Na₂SO₄, and was washed with 1,4-dioxane (2 mL). The combined 1,4-dioxane layers were concentrated in vacuo and the residue was purified by reverse phase HPLC (20-55% CH₃CN/water with 0.1% TFA). The product was diluted with 1:1 THF:CH₃CN (6 mL), treated with MP-carbonate (250 mg, 2.7 mmol/g), filtered after 1 h at room temperature, concentrated in vacuo, and lyophilized to afford the title compound (38 mg, 35%). MS(ES)⁺ m/e 413.3 [M+H]⁺.

### Example 59

### 5-[4-(4-{[(3S)-1-(cyclopropylcarbonyl)-3-pyrrolidinyl]methyl}-4H-1,2,4-triazol-3-yl)phenyl]-1H-indole

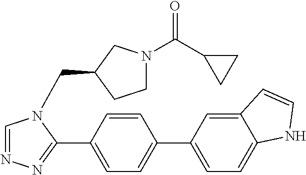 [SMILES: O=C(C1CC1)N1CC[C@@H](Cn2cnnc2-c2ccc(-c3ccc4[nH]ccc4c3)cc2)C1]

A mixture of 3-(4-bromophenyl)-4-{[(3S)-1-(cyclopropylcarbonyl)-3-pyrrolidinyl]methyl}-4H-1,2,4-triazole (100 mg, 0.266 mmol), 5-indole-1H-boronic acid pinacol ester (70 mg, 0.288 mg), and PdCl₂(dppf) (20 mg, 0.024 mmol) in 1,4-dioxane (2 mL) and 2 M aq. K₂CO₃ (1 mL) was heated at 110° C. for 1 h. The mixture was filtered through a plug of Celite® and Na₂SO₄ and washed with 1,4-dioxane (4 mL). The combined 1,4-dioxane layers were concentrated in vacuo and the residue was purified by reverse phase HPLC (20-50% CH₃CN/water with 0.1% TFA). The combined product fractions were neutralized with saturated aq. NaHCO₃ and the organics were extracted with CH₂Cl₂. The organic layer was separated, dried over Na₂SO₄, filtered, and concentrated in vacuo to afford the title compound (75 mg, 68%). MS(ES)⁺ m/e 412.0 [M+H]⁺.

### Example 60

### 5-[4-(4-{[(3S)-1-(cyclopropylcarbonyl)-3-pyrrolidinyl]methyl}-4H-1,2,4-triazol-3-yl)phenyl]-1H-indazole

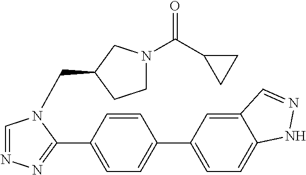 [SMILES: O=C(C1CC1)N1CC[C@@H](Cn2cnnc2-c2ccc(-c3ccc4[nH]ncc4c3)cc2)C1]

Following the procedure described in Example 59a with 5-(4,4,5,5-tetramethyl-1,3,2-dioxaborolan-2-yl)-1H-indazole for 2 h at 120° C. and purification by reverse phase HPLC (10-70% CH₃CN/water with 0.1% NH₄OH) gave the title compound (60%) as a solid. LCMS (ES⁺) m/z 413.0 [M+H]⁺.

### Example 61

### 4-{[(3S)-1-(cyclopropylcarbonyl)-3-pyrrolidinyl]methyl}-3-[4′-(methyloxy)-4-biphenylyl]-4H-1,2,4-triazole

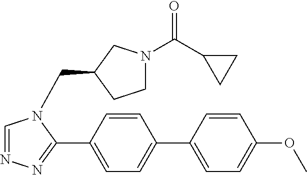 [SMILES: COc1ccc(-c2ccc(-c3nncn3C[C@@H]3CCN(C(=O)C4CC4)C3)cc2)cc1]

Following the procedure described in Example 58g with 4,4,5,5-tetramethyl-2-[4-(methyloxy)phenyl]-1,3,2-dioxaborolane (1.026 eq) afforded the title compound (30%) as a solid. LCMS (ES⁺) m/z 403.1 [M+H]⁺.

### Example 62

### 4′-(4-{[(3S)-1-(cyclopropylcarbonyl)-3-pyrrolidinyl]methyl}-4H-1,2,4-triazol-3-yl)-4-biphenylcarbonitrile

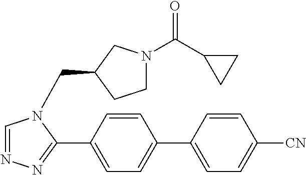 [SMILES: N#Cc1ccc(-c2ccc(-c3nncn3C[C@@H]3CCN(C(=O)C4CC4)C3)cc2)cc1]

Following the procedure described in Example 58g with 4-cyanophenylboronic acid (1.022 eq) afforded the title compound (46%) as a solid. LCMS (ES⁺) m/z 398.0 [M+H]⁺.

### Example 63

### 4-{[(3S)-1-(cyclopropylcarbonyl)-3-pyrrolidinyl]methyl}-3-(2′,4′-difluoro-4-biphenylyl)-4H-1,2,4-triazole

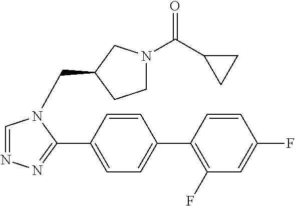 [SMILES: O=C(C1CC1)N1CC[C@@H](Cn2cnnc2-c2ccc(-c3ccc(F)cc3F)cc2)C1]

Following the procedure described in Example 58g with 2,4-difluorophenylboronic acid (1.069 eq) afforded the title compound (44%) as a solid. LCMS (ES⁺) m/z 409.3 [M+H]⁺.

### Example 64

### 4′-(4-{[(3S)-1-(cyclopropylcarbonyl)-3-pyrrolidinyl]methyl}-4H-1,2,4-triazol-3-yl)-2-biphenylcarbonitrile

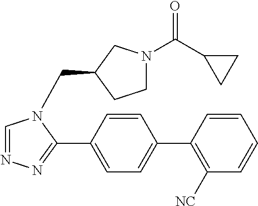 [SMILES: N#Cc1ccccc1-c1ccc(-c2nncn2C[C@@H]2CCN(C(=O)C3CC3)C2)cc1]

A mixture of 3-(4-bromophenyl)-4-{[(3S)-1-(cyclopropylcarbonyl)-3-pyrrolidinyl]methyl}-4H-1,2,4-triazole (150 mg, 0.400 mmol), 2-cyanoboronic acid (60 mg, 0.408 mmol), PdCl₂(dppf) (30 mg, 0.037 mmol), and K₂CO₃ (550 mg, 3.98 mmol) in 1,4-dioxane (3 mL) was stirred at 100° C. for 72 h. Analysis by LCMS revealed the reaction had only progressed to 50% completion. After another 72 h (no change in reaction progress), the mixture was passed through a plug of Celite® and Na₂SO₄, rinsing with 1,4-dioxane (2 mL). The 1,4-dioxane filtrates were combined and concentrated in vacuo, and the residue was purified by reverse phase HPLC (15-50% CH₃CN/water with 0.1% TFA). The product was diluted with 1:1 THF:CH₃CN (6 mL), treated with MP-carbonate (2.7 mmol/g), filtered after 2 h at room temperature, and concentrated in vacuo to afford the title compound (60 mg, 38%). MS(ES)⁺ m/e 398.0 [M+H]⁺.

### Example 65

### 6-[4-(4-{[(3S)-1-(cyclopropylcarbonyl)-3-pyrrolidinyl]methyl}-4H-1,2,4-triazol-3-yl)phenyl]-1H-pyrrolo[3,2-b]pyridine

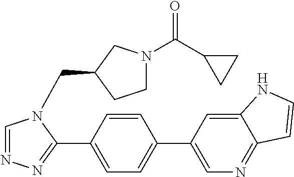 [SMILES: O=C(C1CC1)N1CC[C@@H](Cn2cnnc2-c2ccc(-c3cnc4cc[nH]c4c3)cc2)C1]

### a) 6-bromo-1-({[2-(trimethylsilyl)ethyl]oxy}methyl)-1H-pyrrolo[3,2-b]pyridine

To a solution of 6-bromo-1H-pyrrolo[3,2-b]pyridine (200 mg, 1.015 mmol) in DMF (2 mL) was added NaH (61 mg, 1.52 mmol). After 30 min, {2-[(chloromethyl)oxy]ethyl} (trimethyl)silane (0.270 mL, 1.523 mmol) was added and the reaction was stirred at 25° C. for 3 days. Analysis by LCMS indicated the reaction had gone to completion. Brine and EtOAc were added and the layers were separated. The organic layer was washed with 1:1 water:brine (7×) to remove excess DMF. The organic layer was dried over Na₂SO₄, filtered, and concentrated in vacuo. Purification of the residue by flash chromatography (0-50% EtOAc/hexanes) provided the title compound (230 mg, 67%). MS(ES)⁺ m/e 329.2 [M+H]⁺.

### b) 6-[4-(4-{[(3S)-1-(cyclopropylcarbonyl)-3-pyrrolidinyl]methyl}-4H-1,2,4-triazol-3-yl)phenyl]-1-({[2-(trimethylsilyl)ethyl]oxy}methyl)-1H-pyrrolo[3,2-b]pyridine

A mixture of 6-bromo-1-({[2-(trimethylsilyl)ethyl]oxy}methyl)-1H-pyrrolo[3,2-b]pyridine (85 mg, 0.260 mmol), bis(pinacolato)diboron (66 mg, 0.260 mmol), KOAc (100 mg, 1.019 mmol), and PdCl₂(dppf) (20 mg, 0.024 mmol) in 1,4-dioxane (2 mL) was stirred at 100° C. for 5 h. Analysis by LCMS revealed the desired boronic ester present as 90% of reaction mixture. To this was added 3-(4-bromophenyl)-4-{[1-(cyclopropylcarbonyl)-3-pyrrolidinyl]methyl}-4H-1,2,4-triazole (97 mg, 0.260 mmol), and the reaction mixture was stirred at 100° C. overnight. Analysis by LCMS analysis indicated triazole bromide, but no boronate, present. The mixture was added to EtOAc and then washed with water. The organic layer was separated, dried over Na₂SO₄, and concentrated in vacuo. Purification of the residue by flash chromatography (0-10% MeOH/CH₂Cl₂) afforded the title compound (30 mg, 21%). MS(ES)⁺ m/e 543.4 [M+H]⁺.

### c) 6-[4-(4-{[(3S)-1-(cyclopropylcarbonyl)-3-pyrrolidinyl]methyl}-4H-1,2,4-triazol-3-yl)phenyl]-1H-pyrrolo[3,2-b]pyridine

To 6-[4-(4-{[(3S)-1-(cyclopropylcarbonyl)-3-pyrrolidinyl]methyl}-4H-1,2,4-triazol-3-yl)phenyl]-1-({[2-(trimethylsilyl)ethyl]oxy}methyl)-1H-pyrrolo[3,2-b]pyridine (30 mg, 0.055 mmol) was added 6 N aq. HCl (1 mL, 0.055 mmol). The reaction was stirred overnight at room temperature, at which point analysis by LCMS indicated complete deprotection of the starting material to the N-methyl alcohol. The reaction was concentrated in vacuo and the residue was taken up in MeOH (4 mL) and Et₃N (0.200 mL, 1.435 mmol) and stirred at reflux (90° C.) for 30 min. The reaction mixture was concentrated in vacuo and the residue was purified by flash chromatography (5-20% MeOH/CH₂Cl₂) and then reverse phase HPLC (10-90% CH₃CN/water with 0.1% NH₄OH) to afford the title compound (19 mg, 83%). MS(ES)⁺ m/e 413.3 [M+H]⁺.

### Example 66

### 4-[4-(4-{[(3S)-1-(cyclopropylcarbonyl)-3-pyrrolidinyl]methyl}-4H-1,2,4-triazol-3-yl)phenyl]-1H-indole

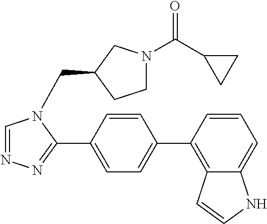 [SMILES: O=C(C1CC1)N1CC[C@@H](Cn2cnnc2-c2ccc(-c3cccc4[nH]ccc34)cc2)C1]

A mixture of 3-(4-bromophenyl)-4-{[(3S)-1-(cyclopropylcarbonyl)-3-pyrrolidinyl]methyl}-4H-1,2,4-triazole (100 mg, 0.266 mmol), indole-4-boronic acid (45 mg, 0.28 mmol), and PdCl₂(dppf) (20 mg, 0.092 mmol) in 1,4-dioxane (2 mL) and 2 M aq. K₂CO₃ (1 mL) was stirred at 100° C. for 1 h. The reaction mixture was cooled, and the 1,4-dioxane layer was passed through a plug of Celite® and Na₂SO₄ while being rinsed with 1,4-dioxane (4 mL). The 1,4-dioxane filtrates were combined and concentrated in vacuo. Purification of the residue by reverse phase HPLC (10-90% CH₃CN/water with 0.1% NH₄OH) gave the title compound (75 mg, 68%). MS(ES)⁺ m/e 412.2 [M+H]⁺.

### Example 67

### 4-[4-(4-{[(3S)-1-(cyclopropylcarbonyl)-3-pyrrolidinyl]methyl}-4H-1,2,4-triazol-3-yl)phenyl]-1H-indazole

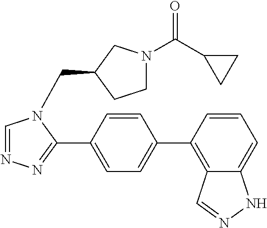 [SMILES: O=C(C1CC1)N1CC[C@@H](Cn2cnnc2-c2ccc(-c3cccc4[nH]ncc34)cc2)C1]

A mixture of 4-bromo-1-(phenylsulfonyl)-1H-indazole (55 mg, 0.163 mmol), bis(pinacolato)diboron (45 mg, 0.177 mmol), KOAc (64.0 mg, 0.652 mmol), and PdCl₂(dppf) (20 mg, 0.024 mmol) in 1,4-dioxane (2 mL) was stirred at 110° C. for 2 h. 3-(4-Bromophenyl)-4-{[(3S)-1-(cyclopropylcarbonyl)-3-pyrrolidinyl]methyl}-4H-1,2,4-triazole (61 mg, 0.163 mmol) and 2 M aq. K₂CO₃ (1 mL) were added and the reaction mixture was stirred at 110° C. The 1,4-dioxane layer was removed, passed through a plug of Celite® and Na₂SO₄, rinsed with 1,4-dioxane (4 mL), and concentrated in vacuo. Purification of the residue by reverse phase HPLC (10-90% CH₃CN/water with 0.1% NH₄OH) provided the title compound (7 mg, 10%). MS(ES)⁺ m/e 413.2 [M+H]⁺.

### Example 68

### 7-[4-(4-{[(3S)-1-(cyclopropylcarbonyl)-3-pyrrolidinyl]methyl}-4H-1,2,4-triazol-3-yl)phenyl]imidazo[1,2-a]pyridine

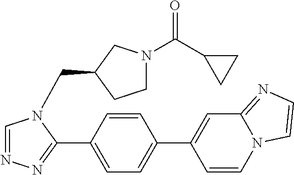 [SMILES: O=C(C1CC1)N1CC[C@@H](Cn2cnnc2-c2ccc(-c3ccn4ccnc4c3)cc2)C1]

A mixture of 7-bromoimidazo[1,2-a]pyridine (125 mg, 0.634 mmol), bis(pinacolato)diboron (180 mg, 0.709 mmol), KOAc (250 mg, 2.55 mmol), and PdCl₂(dppf) (50 mg, 0.061 mmol) in 1,4-dioxane (3 mL) was stirred at 100° C. 3-(4-Bromophenyl)-4-{[(3S)-1-(cyclopropylcarbonyl)-3-pyrrolidinyl]methyl}-4H-1,2,4-triazole (230 mg, 0.613 mmol) and 2 M aq. K₂CO₃ (1.5 mL) were added and the reaction mixture was stirred at 100° C. overnight. The 1,4-dioxane layer was removed, passed through a plug of Celite® and Na₂SO₄, rinsed with 1,4-dioxane (4 mL), and the organic layers concentrated in vacuo. Purification of the residue by reverse phase HPLC (5-45% CH₃CN/water with 0.1% TFA), reverse phase HPLC (10-70% CH₃CN/water with 0.1% NH₄OH), and then reverse phase HPLC (2-40% MeOH/water with 0.08% NH₄OH) provided the title compound (36 mg, 13%). MS(ES)⁺ m/e 413.3 [M+H]⁺.

### Example 69

### N′-[4′-(4-{[(3S)-1-(cyclopropylcarbonyl)-3-pyrrolidinyl]methyl}-4H-1,2,4-triazol-3-yl)-3-biphenylyl]-N,N-dimethylsulfamide

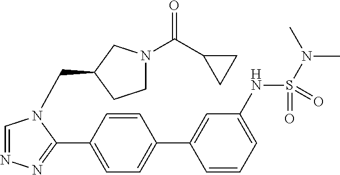 [SMILES: CN(C)S(=O)(=O)Nc1cccc(-c2ccc(-c3nncn3C[C@@H]3CCN(C(=O)C4CC4)C3)cc2)c1]

Following the procedure described in Example 66a with 3-(N,N-dimethylsulfamoylamino)phenylboronic acid (1.0 eq) afforded the title compound (40%) as a solid. LCMS (ES⁺) m/z 495.3 [M+H]⁺.

### Example 70

### 6-[4-(4-{[(3S)-1-(cyclopropylcarbonyl)-3-pyrrolidinyl]methyl}-4H-1,2,4-triazol-3-yl)-3-fluorophenyl]-1H-indole

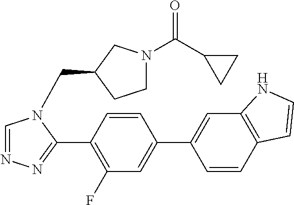 [SMILES: O=C(C1CC1)N1CC[C@@H](Cn2cnnc2-c2ccc(-c3ccc4cc[nH]c4c3)cc2F)C1]

### a) 1,1-dimethylethyl 2-[(4-bromo-2-fluorophenyl)carbonyl]hydrazinecarboxylate

To a mixture of 2-fluoro-4-bromobenzoic acid (5 g, 22.83 mmol) and tert-butylcarbazate (3.02 g, 22.83 mmol) in DMF (100 mL) was added 1-hydroxy-7-azabenzotriazole (0.311 g, 2.283 mmol) and then EDC (5.25 g, 27.4 mmol). After stirring for 2 h at room temperature, the reaction was poured into water (300 mL) and stirred for 20 min as a solid precipitated. The solid was filtered, washed with water (200 mL), and dried in vacuo to afford the title compound (5.53 g, 73%). MS(ES)⁺ m/e 333.9, 335.0 [M+H]⁺.

### b) 4-bromo-2-fluorobenzohydrazide

To a solution of 1,1-dimethylethyl 2-[(4-bromo-2-fluorophenyl)carbonyl]hydrazinecarboxylate (5.53 g, 16.60 mmol) in 1,4-dioxane (100 mL) was added 4 N HCl in 1,4-dioxane (41.5 mL, 166 mmol). The reaction mixture was stirred at room temperature under a nitrogen bubbler until the reaction had progressed ˜80% (1 day), during which time a white precipitate had formed. Additional 1,4-dioxane (50 mL) and 4 N HCl in 1,4-dioxane (20 mL) were added and the reaction was stirred overnight, at which point analysis by LCMS indicated complete progression to product. The reaction was concentrated in vacuo and the solid dried to constant weight to afford the title compound (4.65 g, 100%) as the hydrochloride salt. MS(ES)⁺ m/e 232.7, 234.8 [M+H]⁺.

### c) 1,1-dimethylethyl (3S)-3-{[3-(4-bromo-2-fluorophenyl)-4H-1,2,4-triazol-4-yl]methyl}-1-pyrrolidinecarboxylate

A solution of 4-bromo-2-fluorobenzohydrazide (1.4 g, 5.19 mmol) in CH₂Cl₂ (15 mL) and N,N-dimethylformamide dimethylacetal (1.4 mL, 10.46 mmol) was stirred at reflux (bath temp 100° C.) for 30 min and then concentrated in vacuo. The residue was dissolved in warm THF (10 mL), added to 1,1-dimethylethyl (3S)-3-(aminomethyl)-1-pyrrolidinecarboxylate (1.040 g, 5.19 mmol), and stirred overnight at 110° C. After 24 h, the reaction mixture was cooled to room temperature and concentrated in vacuo. Purification of the residue by flash chromatography (0-10% MeOH/EtOAc) afforded the title compound (1.0 g, 41%). MS(ES)⁺ m/e 424.9, 427.0 [M+H]⁺.

### d) 3-(4-bromo-2-fluorophenyl)-4-{[(3S)-1-(cyclopropylcarbonyl)-3-pyrrolidinyl]methyl}-4H-1,2,4-triazole

A solution of 1,1-dimethylethyl (3S)-3-{[3-(4-bromo-2-fluorophenyl)-4H-1,2,4-triazol-4-yl]methyl}-1-pyrrolidinecarboxylate (900 mg, 2.116 mmol) in CH₂Cl₂ (10 mL) and TFA (2 mL, 26.0 mmol) was stirred at room temperature under a nitrogen bubbler for 2 h and then concentrated in vacuo. The crude material was dissolved in CH₂Cl₂ (10 mL) and cooled to 0° C. in an ice bath. DIPEA (1.5 mL, 8.59 mmol) was added, the solution was stirred for 2 min, and then a solution of cyclopropancarbonyl chloride (200 μl, 2.185 mmol) in CH₂Cl₂ (2 mL) was added dropwise to the reaction. The reaction mixture was stirred under nitrogen while the ice bath was allowed to warm to room temperature. After 2 h, the reaction mixture was diluted with CH₂Cl₂, washed with saturated aq. NaHCO₃ and then with 1:1 water:brine. The organic layer was separated, dried over Na₂SO₄, filtered, and concentrated in vacuo. Purification of the residue by reverse phase HPLC (10-50% CH₃CN/water with 0.1% TFA), collection of the desired fractions, neutralization with saturated aq. NaHCO₃, extraction with CH₂Cl₂, separation of the layers, and further processing of the organic layer (drying over Na₂SO₄, filtering, and concentrating in vacuo) afforded the title compound (350 mg, 42%). MS(ES)⁺ m/e 393.0, 394.7 [M+H]⁺.

### e) 6-[4-(4-{[(3S)-1-(cyclopropylcarbonyl)-3-pyrrolidinyl]methyl}-4H-1,2,4-triazol-3-yl)-3-fluorophenyl]-1H-indole

A mixture of 3-(4-bromo-2-fluorophenyl)-4-{[(3S)-1-(cyclopropylcarbonyl)-3-pyrrolidinyl]methyl}-4H-1,2,4-triazole (49 mg, 0.125 mmol), indole-6-boronic acid (22 mg, 0.137 mmol), and PdCl₂(dppf) (10 mg, 0.012 mmol) in 1,4-dioxane (2 mL) and 2 M aq. K₂CO₃ (1 mL) was stirred at 100° C. for 1 h. The reaction mixture was cooled to room temperature and the 1,4-dioxane layer was passed through a plug of Celite® and Na₂SO₄, rinsing with 1,4-dioxane (2 mL). The combined 1,4-dioxane layers were concentrated in vacuo and the residue was purified by reverse phase HPLC (20-50% CH₃CN/water with 0.1% TFA). The desired fractions were collected, neutralized with saturated aq. NaHCO₃, extracted with CH₂Cl₂, and the organic layer was dried over Na₂SO₄, filtered, and concentrated in vacuo. Purification of the residue by reverse phase HPLC (10-90% CH₃CN/water with 0.1% NH₄OH) afforded the title compound (18 mg, 33%) as a solid. MS(ES)⁺ m/e 430.1[M+H]⁺.

### Example 71

### 3-[4-(1-benzofuran-5-yl)-2-fluorophenyl]-4-{[(3S)-1-(cyclopropylcarbonyl)-3-pyrrolidinyl]methyl}-4H-1,2,4-triazole

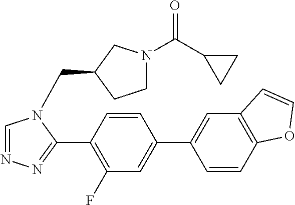 [SMILES: O=C(C1CC1)N1CC[C@@H](Cn2cnnc2-c2ccc(-c3ccc4occc4c3)cc2F)C1]

A mixture of 3-(4-bromo-2-fluorophenyl)-4-{[(3S)-1-(cyclopropylcarbonyl)-3-pyrrolidinyl]methyl}-4H-1,2,4-triazole (59 mg, 0.15 mmol), 5-(4,4,5,5-tetramethyl-1,3,2-dioxaborolan-2-yl)-1-benzofuran (40 mg, 0.164 mmol), and PdCl₂(dppf) (15 mg, 0.018 mmol) in 1,4-dioxane (1 mL) and 2 M aq. K₂CO₃ (0.5 mL) was stirred at 100° C. for 1 h. The reaction mixture was cooled to room temperature and the 1,4-dioxane layer was passed through a plug of Celite® and Na₂SO₄, rinsing with 1,4-dioxane (2 mL). The combined 1,4-dioxane layers were concentrated in vacuo and the residue was purified by reverse phase HPLC (20-60% CH₃CN/water with 0.1% TFA). The desired fractions were collected, neutralized with saturated aq. NaHCO₃, extracted with CH₂Cl₂, and the organic layer was dried over Na₂SO₄, filtered, and concentrated in vacuo to afford the title compound (21 mg, 33%) as a solid. MS(ES)⁺ m/e 431.1 [M+H]⁺.

### Example 72

### 5-[4-(4-{[(3S)-1-(cyclopropylcarbonyl)-3-pyrrolidinyl]methyl}-4H-1,2,4-triazol-3-yl)phenyl]-2,3-dihydro-1H-indole

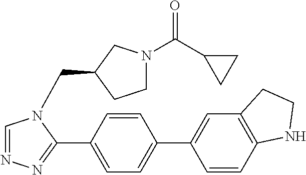 [SMILES: O=C(C1CC1)N1CC[C@@H](Cn2cnnc2-c2ccc(-c3ccc4c(c3)CCN4)cc2)C1]

A mixture of 3-(4-bromophenyl)-4-{[(3S)-1-(cyclopropylcarbonyl)-3-pyrrolidinyl]methyl}-4H-1,2,4-triazole (100 mg, 0.266 mmol), PdCl₂(dppf) (22 mg, 0.027 mmol), bis(pinacolato)diboron (70 mg, 0.276 mmol), and KOAc (100 mg, 1.019 mmol) in 1,4-dioxane (2 mL) was purged with nitrogen and stirred at 100° C. for 5 h. 5-Bromo-1H-indoline (60 mg, 0.303 mmol) and 2 M aq. K₂CO₃ (1.0 mL) were added and the reaction mixture was stirred at 100° C. overnight. The reaction mixture was cooled to room temperature and the 1,4-dioxane layer was passed through a plug of Celite® and Na₂SO₄, rinsing with 1,4-dioxane (4 mL). The combined 1,4-dioxane layers were concentrated in vacuo and the residue was purified by reverse phase HPLC (5-40% CH₃CN/water with 0.1% TFA). The desired fractions were collected, neutralized with saturated aq. NaHCO₃, extracted with CH₂Cl₂, and the organic layer was dried over Na₂SO₄, filtered, and concentrated in vacuo to afford the title compound (34 mg, 31%) as a solid. MS(ES)⁺ m/e 414.1 [M+H]⁺.

### Example 73

### 5-[4-(4-{[(3S)-1-(cyclopropylcarbonyl)-3-pyrrolidinyl]methyl}-4H-1,2,4-triazol-3-yl)phenyl]-1-methyl-1H-pyrrolo[2,3-b]pyridine

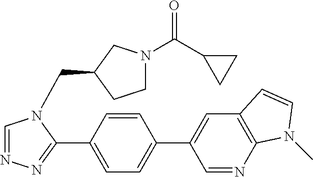 [SMILES: Cn1ccc2cc(-c3ccc(-c4nncn4C[C@@H]4CCN(C(=O)C5CC5)C4)cc3)cnc21]

A mixture of 3-(4-bromophenyl)-4-{[(3S)-1-(cyclopropylcarbonyl)-3-pyrrolidinyl]methyl}-4H-1,2,4-triazole (100 mg, 0.266 mmol), PdCl₂(dppf) (20 mg, 0.024 mmol), bis(pinacolato)diboron (70 mg, 0.276 mmol), and KOAc (100 mg, 1.019 mmol) in 1,4-dioxane (2 mL) was purged with nitrogen and stirred at 110° C. for 4 h. 5-Bromo-1-methyl-1H-pyrrolo[2,3-b]pyridine (60 mg, 0.284 mmol) and 2 M aq. K₂CO₃ (1.0 mL) were added and the reaction mixture was stirred at 110° C. for 1 h. The reaction mixture was cooled to room temperature and the 1,4-dioxane layer was filtered through a plug of Celite® and Na₂SO₄, rinsing with 1,4-dioxane (4 mL). The combined 1,4-dioxane layers were concentrated in vacuo and the residue was purified by reverse phase HPLC (10-40% CH₃CN/water with 0.1% TFA). The desired fractions were collected, neutralized with saturated aq. NaHCO₃, extracted with CH₂Cl₂, and the organic layer was dried over Na₂SO₄, filtered, and concentrated in vacuo. Purification of the residue by reverse phase HPLC (10-90% CH₃CN/water with 0.1% NH₄OH) afforded the title compound (38 mg, 33%) as a solid. MS(ES)⁺ m/e 427.1 [M+H]⁺.

### Example 74

### 4-{[(3S)-1-(cyclopropylcarbonyl)-3-pyrrolidinyl]methyl}-3-[4-(2,3-dihydro-1-benzofuran-5-yl)phenyl]-4H-1,2,4-triazole

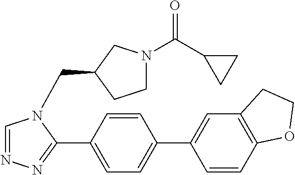 [SMILES: O=C(C1CC1)N1CC[C@@H](Cn2cnnc2-c2ccc(-c3ccc4c(c3)CCO4)cc2)C1]

A mixture of 3-(4-bromophenyl)-4-{[(3S)-1-(cyclopropylcarbonyl)-3-pyrrolidinyl]methyl}-4H-1,2,4-triazole (100 mg, 0.266 mmol), PdCl₂(dppf) (20 mg, 0.024 mmol), bis(pinacolato)diboron (70 mg, 0.276 mmol), and KOAc (100 mg, 1.019 mmol) in 1,4-dioxane (2 mL) was purged with nitrogen and stirred at 110° C. for 4 h. 5-Bromo-2,3-dihydro-1-benzofuran (60 mg, 0.301 mmol) and 2 M aq. K₂CO₃ (1.0 mL) were added and the reaction mixture was stirred at 110° C. for 1 h. The reaction mixture was cooled to room temperature and the 1,4-dioxane layer was filtered through a plug of Celite® and Na₂SO₄, rinsing with 1,4-dioxane (4 mL). The combined 1,4-dioxane layers were concentrated in vacuo and the residue was purified by reverse phase HPLC (20-50% CH₃CN/water with 0.1% TFA). The desired fractions were collected, neutralized with saturated aq. NaHCO₃, extracted with CH₂Cl₂, and the organic layer was dried over Na₂SO₄, filtered, and concentrated in vacuo to afford the title compound (45 mg, 41%) as a solid. MS(ES)⁺ m/e 415.2 [M+H]⁺.

### Example 75

### 5-[4-(4-{[(3S)-1-(cyclopropylcarbonyl)-3-pyrrolidinyl]methyl}-4H-1,2,4-triazol-3-yl)phenyl]-1-methyl-1H-indole

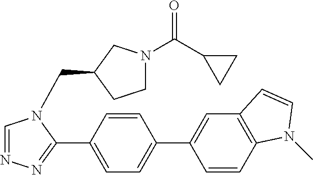 [SMILES: Cn1ccc2cc(-c3ccc(-c4nncn4C[C@@H]4CCN(C(=O)C5CC5)C4)cc3)ccc21]

A mixture of 3-(4-bromophenyl)-4-{[(3S)-1-(cyclopropylcarbonyl)-3-pyrrolidinyl]methyl}-4H-1,2,4-triazole (100 mg, 0.266 mmol), PdCl₂(dppf) (22 mg, 0.027 mmol), bis(pinacolato)diboron (70 mg, 0.276 mmol), and KOAc (100 mg, 1.019 mmol) in 1,4-dioxane (2 mL) was purged with nitrogen and stirred at 100° C. for 5 h. 5-Bromo-1-methyl-1H-indole (60 mg, 0.286 mmol) and 2 M aq. K₂CO₃ (1.0 mL) were added and the reaction mixture was stirred at 100° C. for 1 h. The reaction mixture was cooled to room temperature and the 1,4-dioxane layer was filtered through a plug of Celite® and Na₂SO₄, rinsing with 1,4-dioxane (4 mL). The combined 1,4-dioxane layers were concentrated in vacuo and the residue was purified by reverse phase HPLC (20-50% CH₃CN/water with 0.1% TFA). The desired fractions were collected, neutralized with saturated aq. NaHCO₃, extracted with CH₂Cl₂, and the organic layer was dried over Na₂SO₄, filtered, and concentrated in vacuo to afford the title compound (75 mg, 66%) as a solid. MS(ES)⁺ m/e 426.0 [M+H]⁺.

### Example 76

### 5-[4′-(4-{[(3S)-1-(cyclopropylcarbonyl)-3-pyrrolidinyl]methyl}-4H-1,2,4-triazol-3-yl)-3-biphenylyl]-1H-tetrazole

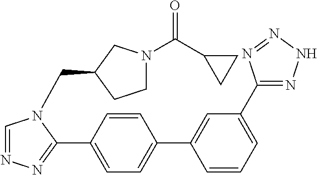 [SMILES: O=C(C1CC1)N1CC[C@@H](Cn2cnnc2-c2ccc(-c3cccc(-c4nn[nH]n4)c3)cc2)C1]

A mixture of 3-(4-bromophenyl)-4-{[(3S)-1-(cyclopropylcarbonyl)-3-pyrrolidinyl]methyl}-4H-1,2,4-triazole (100 mg, 0.266 mmol), 3-(tetrazol-5-yl)phenylboronic acid (60 mg, 0.316 mmol), and PdCl₂(dppf) (20 mg, 0.092 mmol) in 1,4-dioxane (2 mL) and 2 M aq. K₂CO₃ (1.0 mL) was purged with nitrogen and stirred at 100° C. for 1 h. The reaction mixture was cooled to room temperature and the 1,4-dioxane layer was filtered through a plug of Celite® and Na₂SO₄, rinsing with 1,4-dioxane (4 mL). The combined 1,4-dioxane layers were concentrated in vacuo and the residue was purified by reverse phase HPLC (10-25% CH₃CN/water with 0.1% NH₄OH) to afford the title compound (46 mg, 39%) as a solid. MS(ES)⁺ m/e 441.1 [M+H]⁺.

### Example 77

### 6-[4-(4-{[(3S)-1-(cyclopropylcarbonyl)-3-pyrrolidinyl]methyl}-4H-1,2,4-triazol-3-yl)phenyl]-1H-indazole

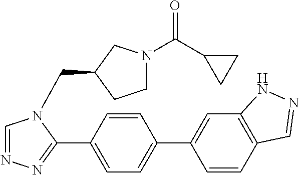 [SMILES: O=C(C1CC1)N1CC[C@@H](Cn2cnnc2-c2ccc(-c3ccc4cn[nH]c4c3)cc2)C1]

A mixture of 3-(4-bromophenyl)-4-{[(3S)-1-(cyclopropylcarbonyl)-3-pyrrolidinyl]methyl}-4H-1,2,4-triazole (100 mg, 0.266 mmol), PdCl₂(dppf) (22 mg, 0.027 mmol), bis(pinacolato)diboron (70 mg, 0.276 mmol), and KOAc (100 mg, 1.019 mmol) in 1,4-dioxane (2 mL) was stirred at 100° C. overnight. 6-Bromo-1H-indazole (55 mg, 0.279 mmol) and 2 M aq. K₂CO₃ (1.0 mL) were added and the reaction mixture was stirred at 100° C. for 72 h. The reaction mixture was cooled to room temperature and the 1,4-dioxane layer was filtered through a plug of Celite® and Na₂SO₄, rinsing with 1,4-dioxane (4 mL). The combined 1,4-dioxane layers were concentrated in vacuo and the residue was purified by reverse phase HPLC (10-70% CH₃CN/water with 0.1% NH₄OH) to afford the title compound (25 mg, 23%) as a solid. MS(ES)⁺ m/e 413.1 [M+H]⁺.

### Example 78

### 5-[4-(4-{[1-(cyclopropylcarbonyl)-3-azetidinyl]methyl}-4H-1,2,4-triazol-3-yl)phenyl]-1H-indazole

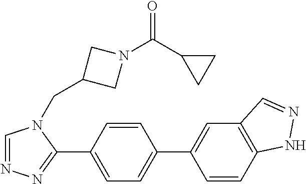 [SMILES: O=C(C1CC1)N1CC(Cn2cnnc2-c2ccc(-c3ccc4[nH]ncc4c3)cc2)C1]

### a) 1,1-dimethylethyl 3-{[3-(4-bromophenyl)-4H-1,2,4-triazol-4-yl]methyl}-1-azetidinecarboxylate

N′-[(4-Bromophenyl)carbonyl]-N,N-dimethylhydrazonoformamide (0.1 g, 0.37 mmol) and 1,1-dimethylethyl 3-(aminomethyl)-1-azetidinecarboxylate (70 mg, 0.376 mmol) were combined in a flask, purged with nitrogen, and stirred at 100° C. for 1 h. THF (1.0 mL) was then added and the reaction mixture was stirred at 100° C. overnight. Analysis by LCMS indicated the reaction had proceed to ˜80% completion. The reaction mixture was concentrated in vacuo and the residue was purified by flash chromatography (0-10% MeOH/EtOAc) to provide the title compound (100 mg, 62%). MS(ES)⁺ m/e 393.0, 394.8 [M+H]⁺.

### b) 5-[4-(4-{[1-(cyclopropylcarbonyl)-3-azetidinyl]methyl}-4H-1,2,4-triazol-3-yl)phenyl]-1H-indazole

A solution of 1,1-dimethylethyl 3-{[3-(4-bromophenyl)-4H-1,2,4-triazol-4-yl]methyl}-1-azetidinecarboxylate (200 mg, 0.509 mmol) in 10% TFA in CH₂Cl₂ (2 mL) was stirred at room temperature for 2 h. The reaction was concentrated in vacuo, and the residue was dissolved in CH₂Cl₂ (2 mL) and cooled to 0° C. in an ice bath. DIPEA (270 μl, 1.55 mmol) was added to the reaction, followed by a solution of cyclopropancarbonyl chloride (55 μl, 0.60 mmol) in CH₂Cl₂ (2 mL), and the reaction mixture was allowed to warm slowly to room temperature. After 2 h, the solution was diluted with CH₂Cl₂ and washed with water. The organic layer was separated, dried over Na₂SO₄, filtered, and concentrated in vacuo. Purification of the residue by reverse phase HPLC (10-40% CH₃CN/water with 0.1% TFA) gave the title compound (140 mg, 69%). MS(ES)⁺ m/e 361.0, 362.9 [M+H]⁺.

### c) 5-[4-(4-{[1-(cyclopropylcarbonyl)-3-azetidinyl]methyl}-4H-1,2,4-triazol-3-yl)phenyl]-1H-indazole

A mixture of 5-[4-(4-{[1-(cyclopropylcarbonyl)-3-azetidinyl]methyl}-4H-1,2,4-triazol-3-yl)phenyl]-1H-indazole (100 mg, 0.277 mmol), 5-(4,4,5,5-tetramethyl-1,3,2-dioxaborolan-2-yl)-1H-indazole (70 mg, 0.287 mmol), and PdCl₂(dppf) (20 mg, 0.024 mmol) in 1,4-dioxane (2 mL) and 2 M aq. K₂CO₃ (1 mL) was purged with nitrogen and stirred at 100° C. for 1 h. The reaction mixture was cooled to room temperature and the 1,4-dioxane layer was filtered through a plug of Celite® and Na₂SO₄, rinsing with 1,4-dioxane (4 mL). The combined 1,4-dioxane layers were concentrated in vacuo and the residue was purified by reverse phase HPLC (10-70% CH₃CN/water with 0.1% NH₄OH) to afford the title compound (25 mg, 22%) as a solid. MS(ES)⁺ m/e 399.1 [M+H]⁺.

### Example 79

### 6-[4-(4-{[1-(cyclopropylcarbonyl)-3-azetidinyl]methyl}-4H-1,2,4-triazol-3-yl)phenyl]-1H-indole

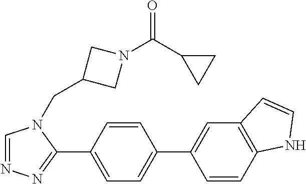 [SMILES: O=C(C1CC1)N1CC(Cn2cnnc2-c2ccc(-c3ccc4[nH]ccc4c3)cc2)C1]

Following the procedure described in Example 78c with indole-6-boronic acid (1.181 eq) afforded the title compound (33 mg, 79%) as a solid. MS(ES)⁺ m/e 398.0 [M+H]⁺.

### Example 80

### 6-[4-(4-{[1-(cyclopropylcarbonyl)-3-azetidinyl]methyl}-4H-1,2,4-triazol-3-yl)phenyl]-1,3-benzothiazole

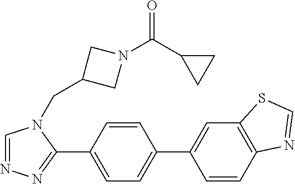 [SMILES: O=C(C1CC1)N1CC(Cn2cnnc2-c2ccc(-c3ccc4ncsc4c3)cc2)C1]

A mixture of 3-(4-bromophenyl)-4-{[1-(cyclopropylcarbonyl)-3-azetidinyl]methyl}-4H-1,2,4-triazole (110 mg, 0.305 mmol), bis(pinacolato)diboron (90 mg, 0.354 mmol), KOAc (120 mg, 1.223 mmol), and PdCl₂(dppf) (25 mg, 0.031 mmol) in 1,4-dioxane (2 mL) was purged with nitrogen and stirred at 100° C. for 1 h. 6-Bromo-1,3-benzothiazole (80 mg, 0.374 mmol) and 2 M aq. K₂CO₃ (1.0 mL) were added and the reaction mixture was stirred at 100° C. for 1 h. The reaction mixture was cooled to room temperature and the 1,4-dioxane layer was filtered through a plug of Celite® and Na₂SO₄, rinsing with 1,4-dioxane (4 mL). The combined 1,4-dioxane layers were concentrated in vacuo and the residue was purified by reverse phase HPLC (10-70% CH₃CN/water with 0.1% NH₄OH) to afford the title compound (20 mg, 16%) as a solid. MS(ES)⁺ m/e 416.2 [M+H]⁺.

### Example 81

### 4-{[(3S)-1-(cyclopropylcarbonyl)-3-pyrrolidinyl]methyl}-3-(3′-methyl-4-biphenylyl)-4H-1,2,4-triazole

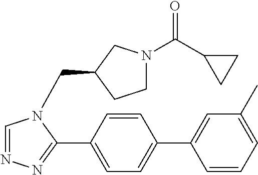 [SMILES: Cc1cccc(-c2ccc(-c3nncn3C[C@@H]3CCN(C(=O)C4CC4)C3)cc2)c1]

A mixture of 3-(4-bromophenyl)-4-{[(3S)-1-(cyclopropylcarbonyl)-3-pyrrolidinyl]methyl}-4H-1,2,4-triazole (100 mg, 0.266 mmol), m-tolylboronic acid (40 mg, 0.294 mmol), and PdCl₂(dppf) (20 mg, 0.024 mmol) in 1,4-dioxane (2 mL) and 2 M aq. K₂CO₃ (1 mL) was purged with nitrogen and stirred at 100° C. for 1 h. The reaction mixture was cooled to room temperature and the 1,4-dioxane layer was diluted with CH₂Cl₂, washed with water, dried over Na₂SO₄, filtered, and concentrated in vacuo. Purification of the residue by reverse phase HPLC (10-90% CH₃CN/water with 0.1% NH₄OH) afforded the title compound (80 mg, 78%) as a solid. MS(ES)⁺ m/e 387.2 [M+H]⁺.

### Example 82

### 4-{[(3S)-1-(cyclopropylcarbonyl)-3-pyrrolidinyl]methyl}-3-(4′-methyl-4-biphenylyl)-4H-1,2,4-triazole

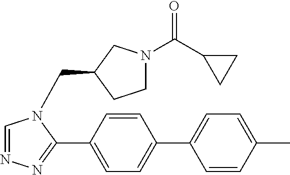 [SMILES: Cc1ccc(-c2ccc(-c3nncn3C[C@@H]3CCN(C(=O)C4CC4)C3)cc2)cc1]

Following the procedure described in Example 81a with p-tolylboronic acid (1.104 eq) afforded the title compound (73 mg, 71%) as a solid. MS(ES)⁺ m/e 387.2 [M+H]⁺.

### Example 83

### 3-(3′-chloro-4-biphenylyl)-4-{[(3S)-1-(cyclopropylcarbonyl)-3-pyrrolidinyl]methyl}-4H-1,2,4-triazole

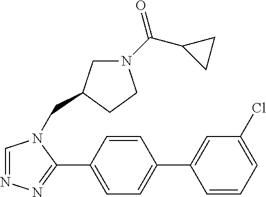 [SMILES: O=C(C1CC1)N1CC[C@@H](Cn2cnnc2-c2ccc(-c3cccc(Cl)c3)cc2)C1]

Following the procedure described in Example 81a with 2-(3-chlorophenyl)-4,4,5,5-tetramethyl-1,3,2-dioxaborolane (1.101 eq) afforded the title compound (60 mg, 55%) as a solid. MS(ES)⁺ m/e 407.2 [M+H]⁺.

### Example 84

### 3-(4′-chloro-4-biphenylyl)-4-{[(3S)-1-(cyclopropylcarbonyl)-3-pyrrolidinyl]methyl}-4H-1,2,4-triazole

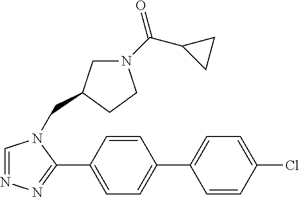 [SMILES: O=C(C1CC1)N1CC[C@@H](Cn2cnnc2-c2ccc(-c3ccc(Cl)cc3)cc2)C1]

Following the procedure described in Example 81a with 2-(4-chlorophenyl)-4,4,5,5-tetramethyl-1,3,2-dioxaborolane afforded the title compound (68 mg, 63%) as a solid. MS(ES)⁺ m/e 407.2 [M+H]⁺.

### Example 85

### 6-[4-(4-{[1-(cyclopropylcarbonyl)-3-azetidinyl]methyl}-4H-1,2,4-triazol-3-yl)-3-fluorophenyl]-1H-indole

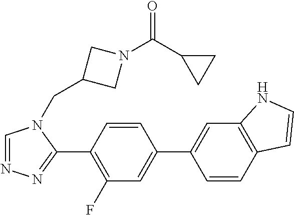 [SMILES: O=C(C1CC1)N1CC(Cn2cnnc2-c2ccc(-c3ccc4cc[nH]c4c3)cc2F)C1]

### a) 1,1-dimethylethyl 3-{[3-(4-bromo-2-fluorophenyl)-4H-1,2,4-triazol-4-yl]methyl}-1-azetidinecarboxylate

A solution of 4-bromo-2-fluorobenzohydrazide (200 mg, 0.742 mmol) in CH₂Cl₂ (2 mL) and N,N-dimethylformamide dimethylacetal (200 μl, 1.494 mmol) was stirred at 50° C. for 30 min. The reaction mixture was concentrated in vacuo, and to the residue was added N-Boc-4-methylamineazetidine (138 mg, 0.742 mmol) and THF (2.0 mL). The reaction was stirred at 100° C. overnight and then concentrated in vacuo. Purification of the residue by flash chromatography (0-10% MeOH/EtOAc) afforded the title compound (120 mg, 36%). MS(ES)⁺ m/e 411.1, 413.0 [M+H]⁺.

### b) 3-(4-bromo-2-fluorophenyl)-4-{[1-(cyclopropylcarbonyl)-3-azetidinyl]methyl}-4H-1,2,4-triazole

To 1,1-dimethylethyl 3-{[3-(4-bromo-2-fluorophenyl)-4H-1,2,4-triazol-4-yl]methyl}-1-azetidinecarboxylate (120 mg, 0.292 mmol) was added a solution of TFA (400 μl, 5.19 mmol) in CH₂Cl₂ (2 mL). The reaction was stirred at room temperature for 2 h and then concentrated in vacuo. The residue was dissolved in CH₂Cl₂ (2 mL) and DIPEA (200 μl, 1.145 mmol) and the resulting solution was cooled to 0° C. A solution of cyclopropancarbonyl chloride (30 μl, 0.328 mmol) in CH₂Cl₂ (2 mL) was added, and the reaction was allowed to slowly warm to room temperature. After 1 h, the reaction mixture was diluted with CH₂Cl₂ and washed with 1:1 water:brine. The organic layer was separated, dried over Na₂SO₄, filtered, and concentrated in vacuo to provide the crude title compound (90 mg, 81%). MS(ES)⁺ m/e 379.2, 381.3 [M+H]⁺.

### c) 6-[4-(4-{[1-(cyclopropylcarbonyl)-3-azetidinyl]methyl}-4H-1,2,4-triazol-3-yl)-3-fluorophenyl]-1H-indole

A mixture of 3-(4-bromo-2-fluorophenyl)-4-{[1-(cyclopropylcarbonyl)-3-azetidinyl]methyl}-4H-1,2,4-triazole (90 mg, 0.237 mmol), indole-6-boronic acid (40 mg, 0.249 mmol), and PdCl₂(dppf) (20 mg, 0.024 mmol) in 1,4-dioxane (2 mL) and 2 M aq. K₂CO₃ (1 mL) was purged with nitrogen and stirred at 100° C. for 1 h. The reaction mixture was cooled to room temperature and the 1,4-dioxane layer was filtered through a plug of Celite® and Na₂SO₄, rinsing with 1,4-dioxane (2 mL). The combined 1,4-dioxane layers were concentrated in vacuo and the residue was purified by reverse phase HPLC (10-50% CH₃CN/water with 0.1% TFA). The desired fractions were collected, neutralized with saturated aq. NaHCO₃, extracted with CH₂Cl₂, and the organic layer was dried over Na₂SO₄, filtered, and concentrated in vacuo. Purification of the residue by reverse phase HPLC (10-65% CH₃CN/water with 0.1% ammonia) afforded the title compound (18 mg, 18%) as a solid. MS(ES)⁺ m/e 416.0 [M+H]⁺.

### Example 86

### 4-{[(3S)-1-(cyclopropylcarbonyl)-3-pyrrolidinyl]methyl}-3-[3-fluoro-4′-(1H-pyrazol-1-yl)-4-biphenylyl]-4H-1,2,4-triazole

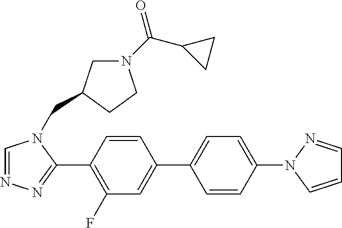 [SMILES: O=C(C1CC1)N1CC[C@@H](Cn2cnnc2-c2ccc(-c3ccc(-n4cccn4)cc3)cc2F)C1]

A mixture of 3-(4-bromo-2-fluorophenyl)-4-{[(3S)-1-(cyclopropylcarbonyl)-3-pyrrolidinyl]methyl}-4H-1,2,4-triazole (100 mg, 0.254 mmol), [4-(1H-pyrazol-1-yl)phenyl]boronic acid (50 mg, 0.266 mmol), and PdCl₂(dppf) (20 mg, 0.024 mmol) in 1,4-dioxane (2 mL) and 2 M aq. K₂CO₃ (1 mL) was purged with nitrogen and stirred at 100° C. for 2 h. The reaction mixture was cooled to room temperature and the 1,4-dioxane layer was diluted with CH₂Cl₂, washed with water, dried over Na₂SO₄, filtered, and concentrated in vacuo. Purification of the residue by reverse phase HPLC (10-80% CH₃CN/water with 0.1% NH₄OH) afforded the title compound (70 mg, 60%) as a solid. MS(ES)⁺ m/e 457.3 [M+H]⁺.

### Example 87

### 4-{[(3S)-1-(cyclopropylcarbonyl)-3-pyrrolidinyl]methyl}-3-[3-fluoro-3′-(1H-pyrazol-5-yl)-4-biphenylyl]-4H-1,2,4-triazole

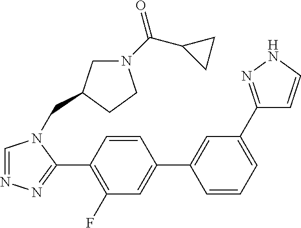 [SMILES: O=C(C1CC1)N1CC[C@@H](Cn2cnnc2-c2ccc(-c3cccc(-c4cc[nH]n4)c3)cc2F)C1]

Following the procedure described in Example 86a with [3-(1H-pyrazol-5-yl)phenyl]boronic acid (1.151 eq) afforded the title compound (68 mg, 59%) as a solid. MS(ES)⁺ m/e 457.2 [M+H]⁺.

### Example 88

### 4′-(4-{[(3S)-1-(cyclopropylcarbonyl)-3-pyrrolidinyl]methyl}-4H-1,2,4-triazol-3-yl)-3-biphenylol

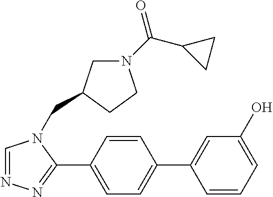 [SMILES: O=C(C1CC1)N1CC[C@@H](Cn2cnnc2-c2ccc(-c3cccc(O)c3)cc2)C1]

Following the procedure described in Example 81a with 3-hydroxyphenlyboronic acid (1.088 eq) afforded the title compound (51 mg, 49%) as a solid. MS(ES)⁺ m/e 389.1 [M+H]⁺.

### Example 89

### 4′-(4-{[(3S)-1-(cyclopropylcarbonyl)-3-pyrrolidinyl]methyl}-4H-1,2,4-triazol-3-yl)-4-biphenylol

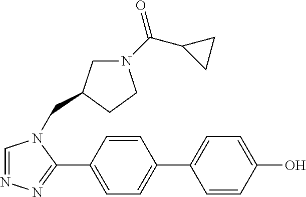 [SMILES: O=C(C1CC1)N1CC[C@@H](Cn2cnnc2-c2ccc(-c3ccc(O)cc3)cc2)C1]

Following the procedure described in Example 81a with 4-hydroxyphenlyboronic acid (1.088 eq) afforded the title compound (45 mg, 44%) as a solid. MS(ES)⁺ m/e 389.1 [M+H]⁺.

### Example 90

### 4-{[(3S)-1-(cyclopropylcarbonyl)-3-pyrrolidinyl]methyl}-3-(2′,3,4′-trifluoro-4-biphenylyl)-4H-1,2,4-triazole

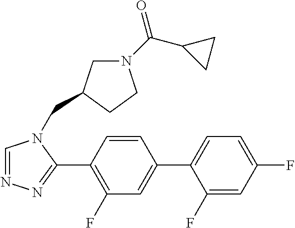 [SMILES: O=C(C1CC1)N1CC[C@@H](Cn2cnnc2-c2ccc(-c3ccc(F)cc3F)cc2F)C1]

A mixture of 3-(4-bromo-2-fluorophenyl)-4-{[(3S)-1-(cyclopropylcarbonyl)-3-pyrrolidinyl]methyl}-4H-1,2,4-triazole (110 mg, 0.280 mmol), 2,4-difluorophenylboronic acid (50 mg, 0.317 mmol), and PdCl₂(dppf) (20 mg, 0.024 mmol) in 1,4-dioxane (2 mL) and 2 M aq. K₂CO₃ (1.0 mL) was stirred at 110° C. for 1 h. The reaction mixture was cooled to room temperature and the 1,4-dioxane layer was diluted with CH₂Cl₂ (20 mL), washed with water, dried over Na₂SO₄, filtered, and concentrated in vacuo. Purification of the residue by reverse phase HPLC (10-75% CH₃CN/water with 0.1% NH₄OH) provided the title compound (65 mg, 54%) as a solid. MS(ES)⁺ m/e 427.0 [M+H]⁺.

### Example 91

### 4-{[(3S)-1-(cyclopropylcarbonyl)-3-pyrrolidinyl]methyl}-3-(3′,3,4′-trifluoro-4-biphenylyl)-4H-1,2,4-triazole

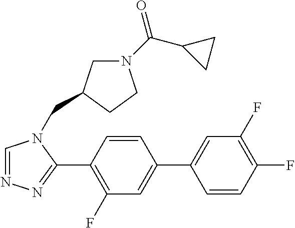 [SMILES: O=C(C1CC1)N1CC[C@@H](Cn2cnnc2-c2ccc(-c3ccc(F)c(F)c3)cc2F)C1]

Following the procedure described in Example 90a with 3,4-difluorophenylboronic acid (1.132 eq) afforded the title compound (71 mg, 59%) as a solid. MS(ES)⁺ m/e 427.0 [M+H]⁺.

### Example 92

### 4-{[(3S)-1-(cyclopropylcarbonyl)-3-pyrrolidinyl]methyl}-3-[3,4′-difluoro-3′-(methyl)-4-biphenylyl]-4H-1,2,4-triazole

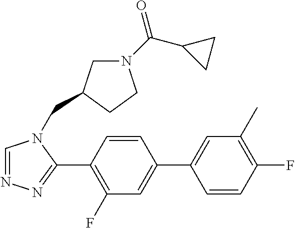 [SMILES: Cc1cc(-c2ccc(-c3nncn3C[C@@H]3CCN(C(=O)C4CC4)C3)c(F)c2)ccc1F]

Following the procedure described in Example 90a with 4-fluoro-3-methylphenylboronic acid (1.15 eq) afforded the title compound (46 mg, 43%) as a solid. MS(ES)⁺ m/e 423.1 [M+H]⁺.

### Example 93

### 4-{[(3S)-1-(cyclopropylcarbonyl)-3-pyrrolidinyl]methyl}-3-[3,4′-difluoro-3′-(methyloxy)-4-biphenylyl]-4H-1,2,4-triazole

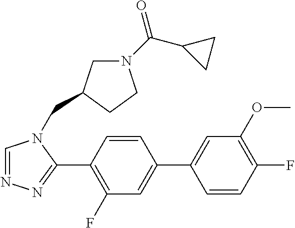 [SMILES: COc1cc(-c2ccc(-c3nncn3C[C@@H]3CCN(C(=O)C4CC4)C3)c(F)c2)ccc1F]

Following the procedure described in Example 90a with 4-fluoro-3-methoxyphenylboronic acid (1.157 eq) afforded the title compound (54 mg, 48%) as a solid. MS(ES)⁺ m/e 439.1 [M+H]⁺.

### Example 94

### 4-{[(3S)-1-(cyclopropylcarbonyl)-3-pyrrolidinyl]methyl}-3-[2′,3-difluoro-4′-(methyloxy)-4-biphenylyl]-4H-1,2,4-triazole

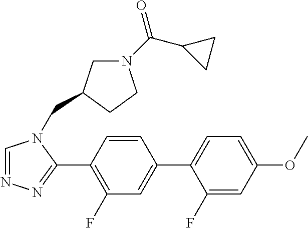 [SMILES: COc1ccc(-c2ccc(-c3nncn3C[C@@H]3CCN(C(=O)C4CC4)C3)c(F)c2)c(F)c1]

Following the procedure described in Example 90a with 2-fluoro-4-methoxyphenylboronic acid (1.157 eq) afforded the title compound (55 mg, 49%) as a solid. MS(ES)⁺ m/e 439.1 [M+H]⁺.

### Example 95

### 4-{[(3S)-1-(cyclopropylcarbonyl)-3-pyrrolidinyl]methyl}-3-[3′,3-difluoro-4′-(methyloxy)-4-biphenylyl]-4H-1,2,4-triazole

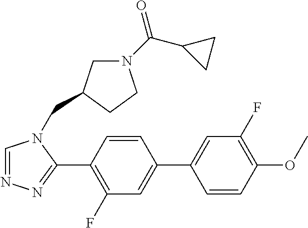 [SMILES: COc1ccc(-c2ccc(-c3nncn3C[C@@H]3CCN(C(=O)C4CC4)C3)c(F)c2)cc1F]

Following the procedure described in Example 90a with 3-fluoro-4-methoxyphenylboronic acid (1.157 eq) afforded the title compound (42 mg, 38%) as a solid. MS(ES)⁺ m/e 439.1 [M+H]⁺.

### Example 96

### 6-[4-(4-{[1-(cyclopropylcarbonyl)-3-azetidinyl]methyl}-4H-1,2,4-triazol-3-yl)-3-fluorophenyl]quinoline

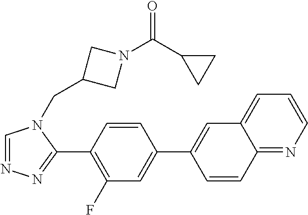 [SMILES: O=C(C1CC1)N1CC(Cn2cnnc2-c2ccc(-c3ccc4ncccc4c3)cc2F)C1]

A mixture of 3-(4-bromo-2-fluorophenyl)-4-{[1-(cyclopropylcarbonyl)-3-azetidinyl]methyl}-4H-1,2,4-triazole (110 mg, 0.290 mmol), 6-(4,4,5,5-tetramethyl-1,3,2-dioxaborolan-2-yl)quinoline (80 mg, 0.314 mmol), and PdCl₂(dppf) (20 mg, 0.024 mmol) in 1,4-dioxane (2 mL) and 2 M aq. K₂CO₃ (1.0 mL) was stirred at 110° C. for 30 min. The reaction mixture was cooled to room temperature and the 1,4-dioxane layer was filtered through a plug of Celite® and Na₂SO₄, rinsing with 1,4-dioxane (2 mL). The combined 1,4-dioxane layers were concentrated in vacuo and the residue was purified by reverse phase HPLC (10-70% CH₃CN/water with 0.1% NH₄OH) and then reverse phase HPLC (5-50% CH₃CN/water with 0.1% ammonia) to afford the title compound (29 mg, 23%) as a solid. MS(ES)⁺ m/e 428.0 [M+H]⁺.

### Example 97

### 4-{[(3S)-1-(cyclopropylcarbonyl)-3-pyrrolidinyl]methyl}-3-(2′,3-difluoro-4′-methyl-4-biphenylyl)-4H-1,2,4-triazole

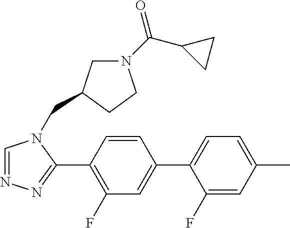 [SMILES: Cc1ccc(-c2ccc(-c3nncn3C[C@@H]3CCN(C(=O)C4CC4)C3)c(F)c2)c(F)c1]

Following the procedure described in Example 90a with 2-fluoro-4-methylphenylboronic acid (1.15 eq) afforded the title compound (51 mg, 48%) as a solid. MS(ES)⁺ m/e 423.0 [M+H]⁺.

### Example 98

### 4-{[(3S)-1-(cyclopropylcarbonyl)-3-pyrrolidinyl]methyl}-3-[3-fluoro-4′-methyl-3′-(methyloxy)-4-biphenylyl]-4H-1,2,4-triazole

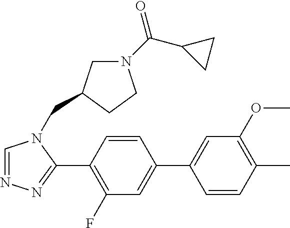 [SMILES: COc1cc(-c2ccc(-c3nncn3C[C@@H]3CCN(C(=O)C4CC4)C3)c(F)c2)ccc1C]

Following the procedure described in Example 90a with 3-methoxy-4-methylphenylboronic acid (1.066 eq) afforded the title compound (46 mg, 42%) as a solid. MS(ES)⁺ m/e 435.2 [M+H]⁺.

### Example 99

### 4-{[(3S)-1-(cyclopropylcarbonyl)-3-pyrrolidinyl]methyl}-3-[3-fluoro-3′-methyl-4′-(methyloxy)-4-biphenylyl]-4H-1,2,4-triazole

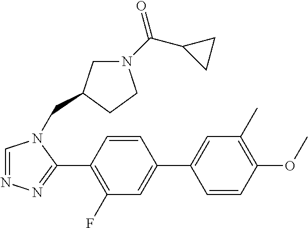 [SMILES: COc1ccc(-c2ccc(-c3nncn3C[C@@H]3CCN(C(=O)C4CC4)C3)c(F)c2)cc1C]

A mixture of 3-(4-bromo-2-fluorophenyl)-4-{[(3S)-1-(cyclopropylcarbonyl)-3-pyrrolidinyl]methyl}-4H-1,2,4-triazole (104 mg, 0.264 mmol), 3-methyl-4-methoxyphenylboronic acid (45 mg, 0.271 mmol), and PdCl₂(dppf) (20 mg, 0.024 mmol) in 1,4-dioxane (2 mL) and 2 M aq. K₂CO₃ (1.0 mL) was stirred at 110° C. for 1 h. The reaction mixture was cooled to room temperature and the 1,4-dioxane layer was diluted with EtOAc (20 mL), washed with 1:1 water:brine, dried over Na₂SO₄, filtered, and concentrated in vacuo. The residue was purified by reverse phase HPLC (10-80% CH₃CN/water with 0.1% NH₄OH) and then reverse phase HPLC (25-55% CH₃CN/water with 0.1% TFA). The desired fractions were collected, neutralized with saturated aq. NaHCO₃, extracted with EtOAc, and the organic layer was dried over Na₂SO₄, filtered, and concentrated in vacuo to afford the title compound (38 mg, 33%) as a solid. MS(ES)⁺ m/e 435.2 [M+H]⁺.

### Example 100

### 3-[3′-chloro-3-fluoro-4′-(methyloxy)-4-biphenylyl]-4-{[(3S)-1-(cyclopropylcarbonyl)-3-pyrrolidinyl]methyl}-4H-1,2,4-triazole

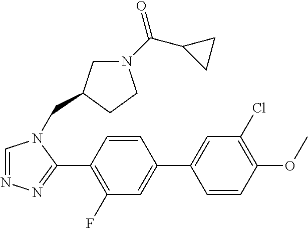 [SMILES: COc1ccc(-c2ccc(-c3nncn3C[C@@H]3CCN(C(=O)C4CC4)C3)c(F)c2)cc1Cl]

Following the procedure described in Example 99a with 4-methoxyl-3-chlorophenylboronic acid (1.014 eq) afforded the title compound (56 mg, 46%) as a solid. MS(ES)⁺ m/e 455.0 [M+H]⁺.

### Example 101

### 7-[4-(4-{[1-(cyclopropylcarbonyl)-3-azetidinyl]methyl}-4H-1,2,4-triazol-3-yl)-3-fluorophenyl]quinoline

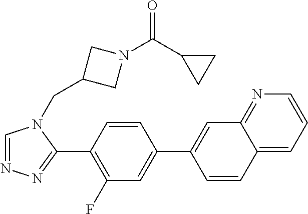 [SMILES: O=C(C1CC1)N1CC(Cn2cnnc2-c2ccc(-c3ccc4cccnc4c3)cc2F)C1]

A mixture of 3-(4-bromo-2-fluorophenyl)-4-{[1-(cyclopropylcarbonyl)-3-azetidinyl]methyl}-4H-1,2,4-triazole (100 mg, 0.264 mmol), 7-(4,4,5,5-tetramethyl-1,3,2-dioxaborolan-2-yl)quinoline (US2007/149513A1; 75 mg, 0.294 mmol), and PdCl₂(dppf) (20 mg, 0.024 mmol) in 1,4-dioxane (2 mL) and 2 M aq. K₂CO₃ (1.0 mL) was stirred at 110° C. for 1 h. The reaction mixture was cooled to room temperature and the 1,4-dioxane layer was filtered through a plug of Celite® and Na₂SO₄, rinsing with 1,4-dioxane (2 mL). The combined 1,4-dioxane layers were concentrated in vacuo and the residue was purified by reverse phase HPLC (10-70% CH₃CN/water with 0.1% NH₄OH) and then reverse phase HPLC (2-40% CH₃CN/water with 0.1% ammonia) to afford the title compound (28 mg, 25%) as a solid. MS(ES)⁺ m/e 428.0 [M+H]⁺.

### Example 102

### 4′-(4-{[(3S)-1-(cyclopropylcarbonyl)-3-pyrrolidinyl]methyl}-4H-1,2,4-triazol-3-yl)-3′,4-difluoro-3-biphenylol

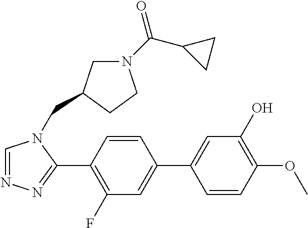 [SMILES: COc1ccc(-c2ccc(-c3nncn3C[C@@H]3CCN(C(=O)C4CC4)C3)c(F)c2)cc1O]

Following the procedure described in Example 99a with 3-hydroxy-4-fluorophenylboronic acid (1.135 eq) afforded the title compound (23 mg, 21%) as a solid. MS(ES)⁺ m/e 424.9 [M+H]⁺.

### Example 103

### 4′-(4-{[(3S)-1-(cyclopropylcarbonyl)-3-pyrrolidinyl]methyl}-4H-1,2,4-triazol-3-yl)-3′-fluoro-4-(methyloxy)-3-biphenylol

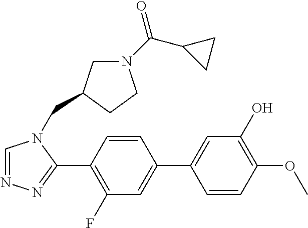 [SMILES: COc1ccc(-c2ccc(-c3nncn3C[C@@H]3CCN(C(=O)C4CC4)C3)c(F)c2)cc1O]

Following the procedure described in Example 99a with 2-(methyloxy)-5-(4,4,5,5-tetramethyl-1,3,2-dioxaborolan-2-yl)phenol (1.022 eq) afforded the title compound (30 mg, 26%) as a solid. MS(ES)⁺ m/e 437.3 [M+H]⁺.

### Example 104

### 4′-(4-{[(3S)-1-(cyclopropylcarbonyl)-3-pyrrolidinyl]methyl}-4H-1,2,4-triazol-3-yl)-3′-fluoro-4-biphenylcarbonitrile

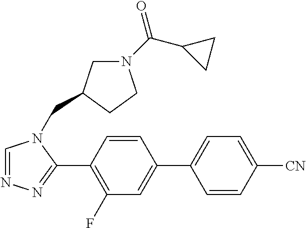 [SMILES: N#Cc1ccc(-c2ccc(-c3nncn3C[C@@H]3CCN(C(=O)C4CC4)C3)c(F)c2)cc1]

Following the procedure described in Example 99a with 4-cyanophenylboronic acid (1.071 eq) afforded the title compound (57 mg, 54%) as a solid. MS(ES)⁺ m/e 416.1 [M+H]⁺.

### Example 105

### 4-{[(3S)-1-(cyclopropylcarbonyl)-3-pyrrolidinyl]methyl}-3-(3,4′-difluoro-4-biphenylyl)-4H-1,2,4-triazole

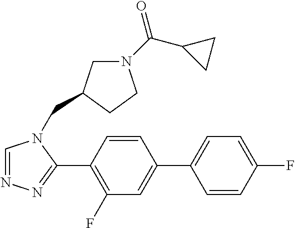 [SMILES: O=C(C1CC1)N1CC[C@@H](Cn2cnnc2-c2ccc(-c3ccc(F)cc3)cc2F)C1]

Following the procedure described in Example 99a with 4-fluorophenylboronic acid (1.124 eq) afforded the title compound (57 mg, 54%) as a solid. MS(ES)⁺ m/e 409.1 [M+H]⁺.

### Example 106

### 4′-(4-{[(3S)-1-(cyclopropylcarbonyl)-3-pyrrolidinyl]methyl}-4H-1,2,4-triazol-3-yl)-3′-fluoro-N,N-dimethyl-4-biphenylamine

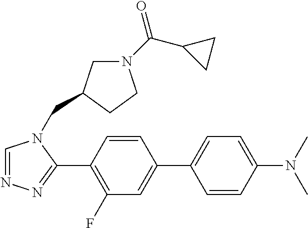 [SMILES: CN(C)c1ccc(-c2ccc(-c3nncn3C[C@@H]3CCN(C(=O)C4CC4)C3)c(F)c2)cc1]

Following the procedure described in Example 99a with 4-dimethylaminophenylboronic acid (1.073 eq) afforded the title compound (63 mg, 57%) as a solid. MS(ES)⁺ m/e 434.2 [M+H]⁺.

### Example 107

### 7-[4-(4-{[(3S)-1-(cyclopropylcarbonyl)-3-pyrrolidinyl]methyl}-4H-1,2,4-triazol-3-yl)-3-fluorophenyl]quinoline

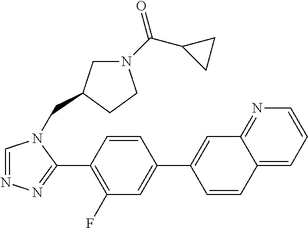 [SMILES: O=C(C1CC1)N1CC[C@@H](Cn2cnnc2-c2ccc(-c3ccc4cccnc4c3)cc2F)C1]

Following the procedure described in Example 99a with 7-(4,4,5,5-tetramethyl-1,3,2-dioxaborolan-2-yl)quinoline (1.079 eq) afforded the title compound (56 mg, 50%) as a solid. MS(ES)⁺ m/e 442.2 [M+H]⁺.

### Example 108

### 3-[4-(4-{[(3S)-1-(cyclopropylcarbonyl)-3-pyrrolidinyl]methyl}-4H-1,2,4-triazol-3-yl)-3-fluorophenyl]quinoline

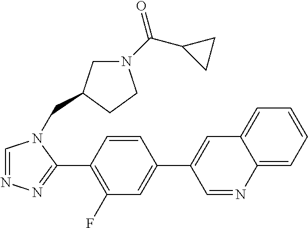 [SMILES: O=C(C1CC1)N1CC[C@@H](Cn2cnnc2-c2ccc(-c3cnc4ccccc4c3)cc2F)C1]

Following the procedure described in Example 99a with 3-quinolineboronic acid (1.137 eq) afforded the title compound (54 mg, 48%) as a solid. MS(ES)⁺ m/e 442.1 [M+H]⁺.

### Example 109

### 4′-(4-{[(3S)-1-(cyclopropylcarbonyl)-3-pyrrolidinyl]methyl}-4H-1,2,4-triazol-3-yl)-3′-fluoro-4-methyl-3-biphenylol

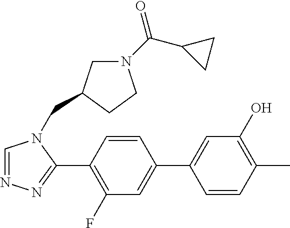 [SMILES: Cc1ccc(-c2ccc(-c3nncn3C[C@@H]3CCN(C(=O)C4CC4)C3)c(F)c2)cc1O]

A mixture of 3-(4-bromo-2-fluorophenyl)-4-{[(3S)-1-(cyclopropylcarbonyl)-3-pyrrolidinyl]methyl}-4H-1,2,4-triazole (100 mg, 0.254 mmol), 3-hydroxy-4-methylphenylboronic acid (40 mg, 0.263 mmol), and PdCl₂(dppf) (20 mg, 0.024 mmol) in 1,4-dioxane (2 mL) and 2 M aq. K₂CO₃ (1.0 mL) was stirred at 110° C. for 1 h. The reaction mixture was cooled to room temperature and the 1,4-dioxane layer was filtered through a plug of Celite® and Na₂SO₄, rinsing with 1,4-dioxane (2 mL). The combined 1,4-dioxane layers were concentrated in vacuo and the residue was purified by reverse phase HPLC (10-75% CH₃CN/water with 0.1% NH₄OH) to afford the title compound (45 mg, 42%) as a solid. MS(ES)⁺ m/e 421.0 [M+H]⁺.

### Biological Assays

### FAS Assay

FAS activity was measured through one of the two following assays.

### Assay #1:

Inhibition of FAS activity can be measured based on the detection of residual NADPH substrate after the FAS assay is quenched. This assay is run as a 10 μL endpoint assay in 384-well format, where the reaction contains 20 μM malonyl-CoA, 2 μM acetyl-CoA, 30 μM NADPH and 40 nM FAS in 50 mM sodium phosphate, pH 7.0. The assay is run by sequentially dispensing 5 μl of a malonyl-CoA solution, then enzyme solution (containing the acetyl-CoA, and NADPH) into a black, low volume assay plate (Greiner 784076) pre-dispensed with 100 nL compound solutions in DMSO. The reaction is incubated at ambient temperature for 60 minutes, then quenched with 5 μL of a developing solution composed of 90 μM resazurin, 0.3 IU/ml diaphorase in 50 mM sodium phosphate, pH 7.0. The developed reaction is read on a Molecular Devices Analyst or Acquest (or equivalent) plate reader using a 530 nm excitation wavelength filter, a 580 nm emission filter, and 561 nm dichroic filter. The test compounds are prepared in neat DMSO at a concentration of 10 mM. For inhibition curves, compounds are diluted using a three fold serial dilution and tested at 11 concentrations (e.g. 25 μM-0.42 nM). Curves are analysed using ActivityBase and XLfit, and results are expressed as pIC50 values.

### Assay #2:

Inhibition of FAS can also be quantified based on the detection of the CoA products with a thio-reactive coumarin dye. This assay is run as a 10 μL endpoint assay in 384-well format, where the reaction contains 20 μM malonyl-CoA, 20 μM acetyl-CoA, 40 μM NADPH and 2 nM FAS in 50 mM sodium phosphate, pH 7.0, and 0.04% Tween-20. The assay is run by adding 5 μL enzyme solution to a black, low volume assay plate (Greiner 784076) pre-dispensed with 100 nl compound solutions in DMSO. After 30 minutes, 5 μL substrate is added, and the reaction incubated at ambient temperature for an additional 60 minutes. The reaction is then quenched with 10 μL of 6M guanidine-HCl containing 50 μM CPM (7-diethylamino-3-(4′-maleimidylphenyl)-4-methylcoumarin (CPM; thio-reactive dye), and incubated for 30 minutes. The plate is read on an Envision (PerkinElmer) or equivalent plate reader using a 380 nm excitation wavelength filter, and a 486 nm emission filter. Data fitting and compound preparations are done as described above.

### Biological Data

Exemplified compounds of the present invention (Examples 1-109) were tested according to the above assays and were found to be inhibitors of FAS. The IC₅₀ values ranged from about 1 to about 2,000 nM; the IC₅₀ values of the preferred compounds ranged from about 1 to about 100 nM. The compounds described below were tested generally according to the assays described herein. The IC₅₀ for each compound was either reported in at least one experiment or the average of multiple experiments.

Example 98: 200 nM

Example 96: 631 nM

Example 87: 63 nM

Example 73: 251 nM

Example 67: 13 nM

Example 54: 602 nM

Example 41: 1524 nM

Example 34: 23 nM

Example 26: 190 nM

Example 15: 596 nM

Example 11: 40 nM

Example 5: 752 nM

### Lipogenesis Assay

Cultured primary human pre-adipocytes (Zen-Bio, Cat#ASCO₆₂₈₀₁) are plated at confluence (3×10⁴ cells/well) in 96-well plates (Costar, Cat#3598) coated with 0.2% gelatin (Sigma, Cat#G-6650) in DMEM/F12 medium (InvitroGen Cat#11330-032) supplemented with 10% heat inactivated fetal bovine serum (InvitroGen, Cat#16000-044). The following day (day 1) the cell differentiation is induced by replacing the seeding medium with the differentiation medium composed of DMEM/F12 medium supplemented with 10% heat inactivated fetal bovine serum, 200 μM 3-isobutyl-1-methylxanthine (Sigma, Cat#I-5879), 20 nM dexamethasone (Sigma, Cat#D-8893), 20 nM GW1929 (Sigma, Cat#G5668) and 20 nM insulin (InvitroGen, Cat#03-0110SA). On day 7, differentiation medium is replaced by the re-feed medium made of DMEM/F12 supplemented with 10% heat inactivated serum and 20 nM insulin. The appropriate concentration of tested compounds and controls are added into this medium at that time. On day 12, the relative amount of cellular triglyceride is estimated by using a Trinder kit (Sigma, Cat#TR0100). Re-feed medium is aspirated and cells are washed with PBS (InvitroGen, Cat#14190-144) and the assay is performed according the kit manufacturer protocol. Briefly, reconstituted solutions A and B are mixed with 0.01% digitonin (Sigma, Cat#D-5628) prior to performing the assay and added onto the cells; plates are incubated at 37° C. for one hour. The absorbance is read at 540 nm. The data is first normalized using the following equation: 100*((UNK−Control 1)/(Control 2−Control 1)) where Control 1 is the Robust Mean of the 0% response control and Control 2 is the Robust Mean of the 100% response control. When multiple dilutions of compounds are tested, pXC50 are calculated from curves using the 4-parameter curve fitting with the following equation: y=(a−d)/(1+(s/c)̂b)+d and with IRLS (Iterative Re-weighted Least Squares) algorithms to weight outliers (Mosteller, F. & Tukey J. W. (1977) Data Analysis and Regression, pp 353-365, Addison-Wesley).

1. A compound of Formula (I): 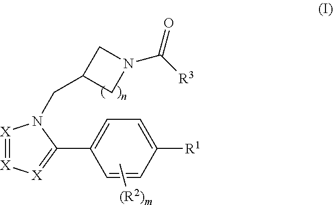
wherein:
R¹ is phenyl, naphthyl, 5- or 6-membered heteroaryl, or 9- or 10-membered heterocyclyl; wherein said phenyl, naphthyl, 5- or 6-membered heteroaryl, or 9- or 10-membered heterocyclyl is optionally substituted 1 to 3 times independently by halogen, (C₁-C₄)alkyl, —CF₃, (C₃-C₇)cycloalkyl, —CO(C₁-C₄)alkyl, —CO(C₃-C₇)cycloalkyl, —CO(phenyl), carboxyl, —CO₂(C₁-C₄)alkyl, —CONR⁵R⁶, phenyl, —SO₂(C₁-C₄)alkyl, —SO₂NR⁵R⁶, cyano, oxo, hydroxyl, (C₁-C₄)alkoxy, (C₃-C₇)cycloalkoxy, hydroxy(C₁-C₄)alkyl-, (C₁-C₄)alkoxy(C₁-C₄)alkyl-, —OCF₃, —NR⁵R⁶, R⁵R⁶N(C₁-C₄)alkyl-, —NHCO(C₁-C₄)alkyl, —NHCONR⁵R⁶, —NHSO₂(C₁-C₄)alkyl, —NHSO₂NR⁵R⁶, or R⁹;
when present each R² is independently selected from the group consisting of halogen, (C₁-C₆)alkyl, hydroxyl, and (C₁-C₄)alkoxy;
R³ is selected from the group consisting of (C₁-C₆)alkyl, —CF₃, (C₃-C₇)cycloalkyl, (C₁-C₄)alkoxy, and —NR⁷R⁸; wherein said (C₁-C₆)alkyl is optionally substituted by hydroxyl, (C₁-C₄)alkoxy, —CF₃, or cyano, and wherein said (C₃-C₇)cycloalkyl is optionally substituted 1 or 2 times independently by halogen, (C₁-C₄)alkyl, hydroxy(C₁-C₄)alkyl-, (C₁-C₄)alkoxy(C₁-C₄)alkyl-, —CF₃, or cyano;
each X is independently N or CR⁴, wherein at least one X is N;
when present each R⁴ is independently hydrogen or (C₁-C₄)alkyl;
R⁵ is selected from the group consisting of hydrogen, (C₁-C₄)alkyl, (C₃-C₇)cycloalkyl, phenyl, and phenyl(C₁-C₃)alkyl-;
R⁶ is hydrogen, (C₁-C₄)alkyl, or (C₃-C₇)cycloalkyl;
or R⁵ and R⁶ taken together with the nitrogen to which they are attached represent a 3- to 7-membered saturated ring optionally containing one other heteroatom which is oxygen, nitrogen, or sulfur, which ring is optionally substituted 1 or 2 times independently by oxo or (C₁-C₄)alkyl;
R⁷ and R⁸ are each independently hydrogen, (C₁-C₄)alkyl, or (C₃-C₇)cycloalkyl;
or R⁷ and R⁸ taken together with the nitrogen to which they are attached represent a 3- to 7-membered saturated ring optionally containing one other heteroatom which is oxygen, nitrogen, or sulfur, which ring is optionally substituted 1 or 2 times independently by oxo or (C₁-C₄)alkyl;
R⁹ is a 5-membered heteroaryl ring containing 1 to 4 heteroatoms selected from oxygen, nitrogen, and sulfur, or a 6-membered heteroaryl ring containing 1 to 3 nitrogen atoms, which 5- or 6-membered ring is optionally substituted 1 or 2 times independently by halogen, (C₁-C₄)alkyl, —CF₃, (C₁-C₄)alkoxy, or —NR⁵R⁶;
m is 0-3; and
n is 1 or 2;
or pharmaceutically acceptable salts thereof.

2. A compound of Formula (I)(A): 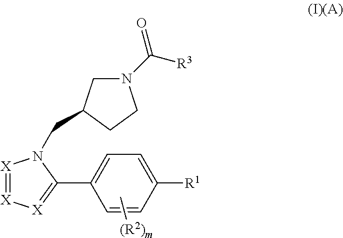
wherein:
R¹ is phenyl, naphthyl, 5- or 6-membered heteroaryl, or 9- or 10-membered heterocyclyl; wherein said phenyl, naphthyl, 5- or 6-membered heteroaryl, or 9- or 10-membered heterocyclyl is optionally substituted 1 to 3 times independently by halogen, (C₁-C₄)alkyl, —CF₃, (C₃-C₇)cycloalkyl, —CO(C₁-C₄)alkyl, —CO(C₃-C₇)cycloalkyl, —CO(phenyl), carboxyl, —CO₂(C₁-C₄)alkyl, —CONR⁵R⁶, phenyl, —SO₂(C₁-C₄)alkyl, —SO₂NR⁵R⁶, cyano, oxo, hydroxyl, (C₁-C₄)alkoxy, (C₃-C₇)cycloalkoxy, hydroxy(C₁-C₄)alkyl-, (C₁-C₄)alkoxy(C₁-C₄)alkyl-, —OCF₃, —NR⁵R⁶, R⁵R⁶N(C₁-C₄)alkyl-, —NHCO(C₁-C₄)alkyl, —NHCONR⁵R⁶, —NHSO₂(C₁-C₄)alkyl, —NHSO₂NR⁵R⁶, or R⁹;
when present each R² is independently selected from the group consisting of halogen, (C₁-C₆)alkyl, hydroxyl, and (C₁-C₄)alkoxy;
R³ is selected from the group consisting of (C₁-C₆)alkyl, —CF₃, (C₃-C₇)cycloalkyl, (C₁-C₄)alkoxy, and —NR⁷R⁸; wherein said (C₁-C₆)alkyl is optionally substituted by hydroxyl, (C₁-C₄)alkoxy, —CF₃, or cyano, and wherein said (C₃-C₇)cycloalkyl is optionally substituted 1 or 2 times independently by halogen, (C₁-C₄)alkyl, hydroxy(C₁-C₄)alkyl-, (C₁-C₄)alkoxy(C₁-C₄)alkyl-, —CF₃, or cyano;
each X is independently N or CR⁴, wherein at least one X is N;
when present each R⁴ is independently hydrogen or (C₁-C₄)alkyl;
R⁵ is selected from the group consisting of hydrogen, (C₁-C₄)alkyl, (C₃-C₇)cycloalkyl, phenyl, and phenyl(C₁-C₃)alkyl-;
R⁶ is hydrogen, (C₁-C₄)alkyl, or (C₃-C₇)cycloalkyl;
or R⁵ and R⁶ taken together with the nitrogen to which they are attached represent a 3- to 7-membered saturated ring optionally containing one other heteroatom which is oxygen, nitrogen, or sulfur, which ring is optionally substituted 1 or 2 times independently by oxo or (C₁-C₄)alkyl;
R⁷ and R⁸ are each independently hydrogen, (C₁-C₄)alkyl, or (C₃-C₇)cycloalkyl;
or R⁷ and R⁸ taken together with the nitrogen to which they are attached represent a 3- to 7-membered saturated ring optionally containing one other heteroatom which is oxygen, nitrogen, or sulfur, which ring is optionally substituted 1 or 2 times independently by oxo or (C₁-C₄)alkyl;
R⁹ is a 5-membered heteroaryl ring containing 1 to 4 heteroatoms selected from oxygen, nitrogen, and sulfur, or a 6-membered heteroaryl ring containing 1 to 3 nitrogen atoms, which 5- or 6-membered ring is optionally substituted 1 or 2 times independently by halogen, (C₁-C₄)alkyl, —CF₃, (C₁-C₄)alkoxy, or —NR⁵R⁶; and
m is 0-3;
or pharmaceutically acceptable salts thereof.

3. A compound of Formula (I)(B): 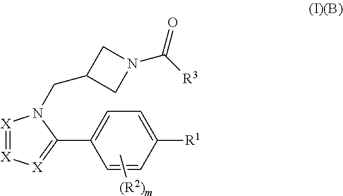
wherein:
R¹ is phenyl, naphthyl, 5- or 6-membered heteroaryl, or 9- or 10-membered heterocyclyl; wherein said phenyl, naphthyl, 5- or 6-membered heteroaryl, or 9- or 10-membered heterocyclyl is optionally substituted 1 to 3 times independently by halogen, (C₁-C₄)alkyl, —CF₃, (C₃-C₇)cycloalkyl, —CO(C₁-C₄)alkyl, —CO(C₃-C₇)cycloalkyl, —CO(phenyl), carboxyl, —CO₂(C₁-C₄)alkyl, —CONR⁵R⁶, phenyl, —SO₂(C₁-C₄)alkyl, —SO₂NR⁵R⁶, cyano, oxo, hydroxyl, (C₁-C₄)alkoxy, (C₃-C₇)cycloalkoxy, hydroxy(C₁-C₄)alkyl-, (C₁-C₄)alkoxy(C₁-C₄)alkyl-, —OCF₃, —NR⁵R⁶, R⁵R⁶N(C₁-C₄)alkyl-, —NHCO(C₁-C₄)alkyl, —NHCONR⁵R⁶, —NHSO₂(C₁-C₄)alkyl, —NHSO₂NR⁵R⁶, or R⁹;
when present each R² is independently selected from the group consisting of halogen, (C₁-C₆)alkyl, hydroxyl, and (C₁-C₄)alkoxy;
R³ is selected from the group consisting of (C₁-C₆)alkyl, —CF₃, (C₃-C₇)cycloalkyl, (C₁-C₄)alkoxy, and —NR⁷R⁸; wherein said (C₁-C₆)alkyl is optionally substituted by hydroxyl, (C₁-C₄)alkoxy, —CF₃, or cyano, and wherein said (C₃-C₇)cycloalkyl is optionally substituted 1 or 2 times independently by halogen, (C₁-C₄)alkyl, hydroxy(C₁-C₄)alkyl-, (C₁-C₄)alkoxy(C₁-C₄)alkyl-, —CF₃, or cyano;
each X is independently N or CR⁴, wherein at least one X is N;
when present each R⁴ is independently hydrogen or (C₁-C₄)alkyl;
R⁵ is selected from the group consisting of hydrogen, (C₁-C₄)alkyl, (C₃-C₇)cycloalkyl, phenyl, and phenyl(C₁-C₃)alkyl-;
R⁶ is hydrogen, (C₁-C₄)alkyl, or (C₃-C₇)cycloalkyl;
or R⁵ and R⁶ taken together with the nitrogen to which they are attached represent a 3- to 7-membered saturated ring optionally containing one other heteroatom which is oxygen, nitrogen, or sulfur, which ring is optionally substituted 1 or 2 times independently by oxo or (C₁-C₄)alkyl;
R⁷ and R⁸ are each independently hydrogen, (C₁-C₄)alkyl, or (C₃-C₇)cycloalkyl;
or R⁷ and R⁸ taken together with the nitrogen to which they are attached represent a 3- to 7-membered saturated ring optionally containing one other heteroatom which is oxygen, nitrogen, or sulfur, which ring is optionally substituted 1 or 2 times independently by oxo or (C₁-C₄)alkyl;
R⁹ is a 5-membered heteroaryl ring containing 1 to 4 heteroatoms selected from oxygen, nitrogen, and sulfur, or a 6-membered heteroaryl ring containing 1 to 3 nitrogen atoms, which 5- or 6-membered ring is optionally substituted 1 or 2 times independently by halogen, (C₁-C₄)alkyl, —CF₃, (C₁-C₄)alkoxy, or —NR⁵R⁶; and
m is 0-3;
or pharmaceutically acceptable salts thereof.

4. The compound or salt according to claim 1, wherein R¹ is phenyl which is optionally substituted 1 to 3 times independently by halogen, (C₁-C₄)alkyl, —CF₃, (C₃-C₇)cycloalkyl, —CO(C₁-C₄)alkyl, —CO(C₃-C₇)cycloalkyl, —CO(phenyl), carboxyl, —CO₂(C₁-C₄)alkyl, —CONR⁵R⁶, phenyl, —SO₂(C₁-C₄)alkyl, —SO₂NR⁵R⁶, cyano, oxo, hydroxyl, (C₁-C₄)alkoxy, (C₃-C₇)cycloalkoxy, hydroxy(C₁-C₄)alkyl-, (C₁-C₄)alkoxy(C₁-C₄)alkyl-, —OCF₃, —NR⁵R⁶, R⁵R⁶N(C₁-C₄)alkyl-, —NHCO(C₁-C₄)alkyl, —NHCONR⁵R⁶, —NHSO₂(C₁-C₄)alkyl, —NHSO₂NR⁵R⁶, or R⁹.

5. The compound or salt according to claim 1, wherein R¹ is furanyl, thienyl, pyrrolyl, imidazolyl, pyrazolyl, triazolyl, tetrazolyl, thiazolyl, oxazolyl, isoxazolyl, oxadiazolyl, thiadiazolyl, isothiazolyl, pyridinyl, pyridazinyl, pyrazinyl, pyrimidinyl, or triazinyl, wherein said furanyl, thienyl, pyrrolyl, imidazolyl, pyrazolyl, triazolyl, tetrazolyl, thiazolyl, oxazolyl, isoxazolyl, oxadiazolyl, thiadiazolyl, isothiazolyl, pyridinyl, pyridazinyl, pyrazinyl, pyrimidinyl, or triazinyl is optionally substituted 1 to 3 times independently by halogen, (C₁-C₄)alkyl, —CF₃, (C₃-C₇)cycloalkyl, —CO(C₁-C₄)alkyl, —CO(C₃-C₇)cycloalkyl, —CO(phenyl), carboxyl, —CO₂(C₁-C₄)alkyl, —CONR⁵R⁶, phenyl, —SO₂(C₁-C₄)alkyl, —SO₂NR⁵R⁶, cyano, oxo, hydroxyl, (C₁-C₄)alkoxy, (C₃-C₇)cycloalkoxy, hydroxy(C₁-C₄)alkyl-, (C₁-C₄)alkoxy(C₁-C₄)alkyl-, —OCF₃, —NR⁵R⁶, R⁵R⁶N(C₁-C₄)alkyl-, —NHCO(C₁-C₄)alkyl, —NHCONR⁵R⁶, —NHSO₂(C₁-C₄)alkyl, —NHSO₂NR⁵R⁶, or R⁹.

6. The compound or salt according to claim 1, wherein R¹ is benzofuranyl, isobenzofuryl, 2,3-dihydrobenzofuryl, 1,3-benzodioxolyl, dihydrobenzodioxinyl, benzothienyl, indolizinyl, indolyl, isoindolyl, indolinyl, isoindolinyl, benzimidazolyl, dihydrobenzimidazolyl, benzoxazolyl, dihydrobenzoxazolyl, benzthiazolyl, benzoisothiazolyl, dihydrobenzoisothiazolyl, indazolyl, pyrrolopyridinyl, pyrrolopyrimidinyl, imidazopyridinyl, imidazopyrimidinyl, pyrazolopyridinyl, pyrazolopyrimidinyl, benzoxadiazolyl, benzthiadiazolyl, benzotriazolyl, triazolopyridinyl, purinyl, quinolinyl, tetrahydroquinolinyl, isoquinolinyl, tetrahydroisoquinolinyl, quinoxalinyl, cinnolinyl, phthalazinyl, quinazolinyl, 1,5-naphthyridinyl, 1,6-naphthyridinyl, 1,7-naphthyridinyl, 1,8-naphthyridinyl, or pteridinyl, wherein said benzofuranyl, isobenzofuryl, 2,3-dihydrobenzofuryl, 1,3-benzodioxolyl, dihydrobenzodioxinyl, benzothienyl, indolizinyl, indolyl, isoindolyl, indolinyl, isoindolinyl, benzimidazolyl, dihydrobenzimidazolyl, benzoxazolyl, dihydrobenzoxazolyl, benzthiazolyl, benzoisothiazolyl, dihydrobenzoisothiazolyl, indazolyl, pyrrolopyridinyl, pyrrolopyrimidinyl, imidazopyridinyl, imidazopyrimidinyl, pyrazolopyridinyl, pyrazolopyrimidinyl, benzoxadiazolyl, benzthiadiazolyl, benzotriazolyl, triazolopyridinyl, purinyl, quinolinyl, tetrahydroquinolinyl, isoquinolinyl, tetrahydroisoquinolinyl, quinoxalinyl, cinnolinyl, phthalazinyl, quinazolinyl, 1,5-naphthyridinyl, 1,6-naphthyridinyl, 1,7-naphthyridinyl, 1,8-naphthyridinyl, or pteridinyl is optionally substituted 1 to 3 times independently by halogen, (C₁-C₄)alkyl, —CF₃, (C₃-C₇)cycloalkyl, —CO(C₁-C₄)alkyl, —CO(C₃-C₇)cycloalkyl, —CO(phenyl), carboxyl, —CO₂(C₁-C₄)alkyl, —CONR⁵R⁶, phenyl, —SO₂(C₁-C₄)alkyl, —SO₂NR⁵R⁶, cyano, oxo, hydroxyl, (C₁-C₄)alkoxy, (C₃-C₇)cycloalkoxy, hydroxy(C₁-C₄)alkyl-, (C₁-C₄)alkoxy(C₁-C₄)alkyl-, —OCF₃, —NR⁵R⁶, R⁵R⁶N(C₁-C₄)alkyl-, —NHCO(C₁-C₄)alkyl, —NHCONR⁵R⁶, —NHSO₂(C₁-C₄)alkyl, —NHSO₂NR⁵R⁶, or R⁹.

7. The compound or salt according to claim 1, wherein:
R² is fluoro, chloro, hydroxyl, methoxy, or methyl; and
m is 1.

8. The compound or salt according to claim 1, wherein R³ is (C₁-C₄)alkyl, —CF₃, (C₃-C₆)cycloalkyl, methoxy, or dimethylamino, wherein said (C₃-C₆)cycloalkyl is optionally substituted 1 or 2 times independently by fluoro or methyl.

9. The compound or salt according to claim 1, wherein R³ is cyclopropyl.

10. The compound or salt according to claim 1, wherein R⁴ is hydrogen or methyl.

11. The compound according to claim 1 which is:
6-[4-(1-{[(3R)-1-(cyclopropylcarbonyl)-3-pyrrolidinyl]methyl}-1H-tetrazol-5-yl)phenyl]-1H-indole;
5-[4-(1-benzofuran-5-yl)phenyl]-1-{[(3R)-1-(cyclopropylcarbonyl)-3-pyrrolidinyl]methyl}-1H-tetrazole;
5-[4-(1-{[(3R)-1-(cyclopropylcarbonyl)-3-pyrrolidinyl]methyl}-1H-tetrazol-5-yl)phenyl]-1H-indole;
1-{[(3R)-1-(cyclopropylcarbonyl)-3-pyrrolidinyl]methyl}-5-(2′,4′-dichloro-4-biphenylyl)-1H-tetrazole;
5-[2′-chloro-4′-(methyloxy)-4-biphenylyl]-1-{[(3R)-1-(cyclopropylcarbonyl)-3-pyrrolidinyl]methyl}-1H-tetrazole;
1-{[(3R)-1-(cyclopropylcarbonyl)-3-pyrrolidinyl]methyl}-5-(4′-fluoro-4-biphenylyl)-1H-tetrazole;
6-[4-(1-{[(3R)-1-(cyclopropylcarbonyl)-3-pyrrolidinyl]methyl}-1H-tetrazol-5-yl)phenyl]-1H-indazole;
6-[4-(1-{[(3R)-1-(cyclopropylcarbonyl)-3-pyrrolidinyl]methyl}-1H-1,2,3-triazol-5-yl)phenyl]-1H-indole;
5-[4-(1-{[(3R)-1-(cyclopropylcarbonyl)-3-pyrrolidinyl]methyl}-1H-1,2,3-triazol-5-yl)phenyl]-1H-indole;
5-[4-(1-benzofuran-5-yl)phenyl]-1-{[(3R)-1-(cyclopropylcarbonyl)-3-pyrrolidinyl]methyl}-1H-1,2,3-triazole;
5-[4-(1-{[(3R)-1-(cyclopropylcarbonyl)-3-pyrrolidinyl]methyl}-1H-1,2,3-triazol-5-yl)phenyl]-1H-indazole;
1-{[(3R)-1-(cyclopropylcarbonyl)-3-pyrrolidinyl]methyl}-5-(2′,4′-dichloro-4-biphenylyl)-1H-1,2,3-triazole;
5-[2′-chloro-4′-(methyloxy)-4-biphenylyl]-1-{[(3R)-1-(cyclopropylcarbonyl)-3-pyrrolidinyl]methyl}-1H-1,2,3-triazole;
6-[4-(4-{[(3S)-1-(cyclopropylcarbonyl)-3-pyrrolidinyl]methyl}-4H-1,2,4-triazol-3-yl)phenyl]-1H-indole;
4-{[(3S)-1-(cyclopropylcarbonyl)-3-pyrrolidinyl]methyl}-3-(2′,4′-dichloro-4-biphenylyl)-5-methyl-4H-1,2,4-triazole;
6-[4-(4-{[(3S)-1-(cyclopropylcarbonyl)-3-pyrrolidinyl]methyl}-5-methyl-4H-1,2,4-triazol-3-yl)phenyl]-1H-indole;
3-[4-(1-benzofuran-5-yl)phenyl]-4-{[(3S)-1-(cyclopropylcarbonyl)-3-pyrrolidinyl]methyl}-5-methyl-4H-1,2,4-triazole;
4-{[(3S)-1-(cyclopropylcarbonyl)-3-pyrrolidinyl]methyl}-3-(2′,4′-dichloro-4-biphenylyl)-4H-1,2,4-triazole;
5-[4-(4-{[(3S)-1-(cyclopropylcarbonyl)-3-pyrrolidinyl]methyl}-4H-1,2,4-triazol-3-yl)phenyl]-1H-indazole;
3-[2′-chloro-4′-(methyloxy)-4-biphenylyl]-4-{[(3S)-1-(cyclopropylcarbonyl)-3-pyrrolidinyl]methyl}-4H-1,2,4-triazole;
5-[4-(1-{[(3S)-1-(cyclopropylcarbonyl)-3-pyrrolidinyl]methyl}-1H-imidazol-2-yl)phenyl]-1H-indole;
6-[4-(1-{[(3S)-1-(cyclopropylcarbonyl)-3-pyrrolidinyl]methyl}-1H-imidazol-2-yl)phenyl]-1H-indole;
2-(3′-chloro-4-biphenylyl)-1-{[(3S)-1-(cyclopropylcarbonyl)-3-pyrrolidinyl]methyl}-1H-imidazole;
1-{[1-(cyclopropylcarbonyl)-3-pyrrolidinyl]methyl}-2-[4′-(methyloxy)-4-biphenylyl]-1H-imidazole;
1-{[1-(cyclopropylcarbonyl)-3-pyrrolidinyl]methyl}-2-(3′-fluoro-4′-methyl-4-biphenylyl)-1H-imidazole;
2-(4-biphenylyl)-1-{[(3S)-1-(cyclopropylcarbonyl)-3-pyrrolidinyl]methyl}-1H-imidazole;
5-[4-(1-{[1-(cyclopropylcarbonyl)-3-pyrrolidinyl]methyl}-1H-imidazol-2-yl)phenyl]-1H-indole;
2-(3′-chloro-4-biphenylyl)-1-{[1-(cyclopropylcarbonyl)-3-pyrrolidinyl]methyl}-1H-imidazole;
2-(4′-chloro-4-biphenylyl)-1-{[(3S)-1-(cyclopropylcarbonyl)-3-pyrrolidinyl]methyl}-1H-imidazole;
1-{[(3S)-1-(cyclopropylcarbonyl)-3-pyrrolidinyl]methyl}-2-(2′,4′-dichloro-4-biphenylyl)-1H-imidazole;
1-{[(3S)-1-(cyclopropylcarbonyl)-3-pyrrolidinyl]methyl}-2-(4′-fluoro-4-biphenylyl)-1H-imidazole;
3-[4-(1-{[(3S)-1-(cyclopropylcarbonyl)-3-pyrrolidinyl]methyl}-1H-imidazol-2-yl)phenyl]pyridine;
6-[4-(1-{[1-(cyclopropylcarbonyl)-3-pyrrolidinyl]methyl}-1H-imidazol-2-yl)phenyl]-1H-indole;
2-[4-(1-benzofuran-5-yl)phenyl]-1-{[(3S)-1-(cyclopropylcarbonyl)-3-pyrrolidinyl]methyl}-1H-imidazole;
1-{[1-(cyclopropylcarbonyl)-3-pyrrolidinyl]methyl}-4,5-dimethyl-2-[4′-(methyloxy)-4-biphenylyl]-1H-imidazole;
1-{[1-(cyclopropylcarbonyl)-3-pyrrolidinyl]methyl}-2-(2′,4′-dichloro-4-biphenylyl)-4,5-dimethyl-1H-imidazole;
2-[2′-chloro-4′-(methyloxy)-4-biphenylyl]-1-{[1-(cyclopropylcarbonyl)-3-pyrrolidinyl]methyl}-4,5-dimethyl-1H-imidazole;
2-(3′-chloro-4′-fluoro-4-biphenylyl)-1-{[1-(cyclopropylcarbonyl)-3-pyrrolidinyl]methyl}-4,5-dimethyl-1H-imidazole;
1-{[1-(cyclopropylcarbonyl)-3-pyrrolidinyl]methyl}-2-(4′-fluoro-3′-methyl-4-biphenylyl)-4,5-dimethyl-1H-imidazole;
1-{[1-(cyclopropylcarbonyl)-3-pyrrolidinyl]methyl}-4,5-dimethyl-2-(4′-methyl-4-biphenylyl)-1H-imidazole;
1-{[1-(cyclopropylcarbonyl)-3-pyrrolidinyl]methyl}-2-(4′-fluoro-4-biphenylyl)-4,5-dimethyl-1H-imidazole;
4′-(1-{[1-(cyclopropylcarbonyl)-3-pyrrolidinyl]methyl}-4,5-dimethyl-1H-imidazol-2-yl)-3-biphenylol;
1-{[1-(cyclopropylcarbonyl)-3-pyrrolidinyl]methyl}-4,5-dimethyl-2-(3′-methyl-4-biphenylyl)-1H-imidazole;
2-(3′-chloro-4-biphenylyl)-1-{[1-(cyclopropylcarbonyl)-3-pyrrolidinyl]methyl}-4,5-dimethyl-1H-imidazole;
2-(4′-chloro-4-biphenylyl)-1-{[1-(cyclopropylcarbonyl)-3-pyrrolidinyl]methyl}-4,5-dimethyl-1H-imidazole;
1-{[1-(cyclopropylcarbonyl)-3-pyrrolidinyl]methyl}-5-methyl-2-(4′-methyl-4-biphenylyl)-1H-imidazole;
1-{[1-(cyclopropylcarbonyl)-3-pyrrolidinyl]methyl}-2-[4′-(ethyloxy)-4-biphenylyl]-5-methyl-1H-imidazole;
1-{[1-(cyclopropylcarbonyl)-3-pyrrolidinyl]methyl}-5-methyl-2-[4′-(methyloxy)-4-biphenylyl]-1H-imidazole;
2-(4′-chloro-4-biphenylyl)-1-{[1-(cyclopropylcarbonyl)-3-pyrrolidinyl]methyl}-5-methyl-1H-imidazole;
1-{[1-(cyclopropylcarbonyl)-3-pyrrolidinyl]methyl}-5-methyl-2-(3′-methyl-4-biphenylyl)-1H-imidazole;
2-(3′-chloro-4-biphenylyl)-1-{[1-(cyclopropylcarbonyl)-3-pyrrolidinyl]methyl}-5-methyl-1H-imidazole;
1-{[1-(cyclopropylcarbonyl)-3-pyrrolidinyl]methyl}-2-(4′-fluoro-4-biphenylyl)-5-methyl-1H-imidazole;
1-{[1-(cyclopropylcarbonyl)-3-pyrrolidinyl]methyl}-2-(2′,4′-dimethyl-4-biphenylyl)-5-methyl-1H-imidazole;
1-{[1-(cyclopropylcarbonyl)-3-pyrrolidinyl]methyl}-2-(2′,4′-dichloro-4-biphenylyl)-5-methyl-1H-imidazole;
1-{[1-(cyclopropylcarbonyl)-3-pyrrolidinyl]methyl}-2-(4′-fluoro-3′-methyl-4-biphenylyl)-5-methyl-1H-imidazole;
2-[2′-chloro-4′-(methyloxy)-4-biphenylyl]-1-{[1-(cyclopropylcarbonyl)-3-pyrrolidinyl]methyl}-5-methyl-1H-imidazole;
2-(3′-chloro-4′-fluoro-4-biphenylyl)-1-{[1-(cyclopropylcarbonyl)-3-pyrrolidinyl]methyl}-5-methyl-1H-imidazole;
3-[4-(1-benzofuran-5-yl)phenyl]-4-{[(3S)-1-(cyclopropylcarbonyl)-3-pyrrolidinyl]methyl}-4H-1,2,4-triazole;
5-[4-(4-{[(3S)-1-(cyclopropylcarbonyl)-3-pyrrolidinyl]methyl}-4H-1,2,4-triazol-3-yl)phenyl]-1H-indole;
5-[4-(4-{[(3S)-1-(cyclopropylcarbonyl)-3-pyrrolidinyl]methyl}-4H-1,2,4-triazol-3-yl)phenyl]-1H-indazole;
4-{[(3S)-1-(cyclopropylcarbonyl)-3-pyrrolidinyl]methyl}-3-[4′-(methyloxy)-4-biphenylyl]-4H-1,2,4-triazole;
4′-(4-{[(3S)-1-(cyclopropylcarbonyl)-3-pyrrolidinyl]methyl}-4H-1,2,4-triazol-3-yl)-4-biphenylcarbonitrile;
4-{[(3S)-1-(cyclopropylcarbonyl)-3-pyrrolidinyl]methyl}-3-(2′,4′-difluoro-4-biphenylyl)-4H-1,2,4-triazole;
4′-(4-{[(3S)-1-(cyclopropylcarbonyl)-3-pyrrolidinyl]methyl}-4H-1,2,4-triazol-3-yl)-2-biphenylcarbonitrile;
6-[4-(4-{[(3S)-1-(cyclopropylcarbonyl)-3-pyrrolidinyl]methyl}-4H-1,2,4-triazol-3-yl)phenyl]-1H-pyrrolo[3,2-b]pyridine;
4-[4-(4-{[(3S)-1-(cyclopropylcarbonyl)-3-pyrrolidinyl]methyl}-4H-1,2,4-triazol-3-yl)phenyl]-1H-indole;
4-[4-(4-{[(3S)-1-(cyclopropylcarbonyl)-3-pyrrolidinyl]methyl}-4H-1,2,4-triazol-3-yl)phenyl]-1H-indazole;
7-[4-(4-{[(3S)-1-(cyclopropylcarbonyl)-3-pyrrolidinyl]methyl}-4H-1,2,4-triazol-3-yl)phenyl]imidazo[1,2-a]pyridine;
N′-[4′-(4-{[(3S)-1-(cyclopropylcarbonyl)-3-pyrrolidinyl]methyl}-4H-1,2,4-triazol-3-yl)-3-biphenylyl]-N,N-dimethylsulfamide;
6-[4-(4-{[(3S)-1-(cyclopropylcarbonyl)-3-pyrrolidinyl]methyl}-4H-1,2,4-triazol-3-yl)-3-fluorophenyl]-1H-indole;
3-[4-(1-benzofuran-5-yl)-2-fluorophenyl]-4-{[(3S)-1-(cyclopropylcarbonyl)-3-pyrrolidinyl]methyl}-4H-1,2,4-triazole;
5-[4-(4-{[(3S)-1-(cyclopropylcarbonyl)-3-pyrrolidinyl]methyl}-4H-1,2,4-triazol-3-yl)phenyl]-2,3-dihydro-1H-indole;
5-[4-(4-{[(3S)-1-(cyclopropylcarbonyl)-3-pyrrolidinyl]methyl}-4H-1,2,4-triazol-3-yl)phenyl]-1-methyl-1H-pyrrolo[2,3-b]pyridine;
4-{[(3S)-1-(cyclopropylcarbonyl)-3-pyrrolidinyl]methyl}-3-[4-(2,3-dihydro-1-benzofuran-5-yl)phenyl]-4H-1,2,4-triazole;
5-[4-(4-{[(3S)-1-(cyclopropylcarbonyl)-3-pyrrolidinyl]methyl}-4H-1,2,4-triazol-3-yl)phenyl]-1-methyl-1H-indole;
5-[4′-(4-{[(3S)-1-(cyclopropylcarbonyl)-3-pyrrolidinyl]methyl}-4H-1,2,4-triazol-3-yl)-3-biphenylyl]-1H-tetrazole;
6-[4-(4-{[(3S)-1-(cyclopropylcarbonyl)-3-pyrrolidinyl]methyl}-4H-1,2,4-triazol-3-yl)phenyl]-1H-indazole;
5-[4-(4-{[1-(cyclopropylcarbonyl)-3-azetidinyl]methyl}-4H-1,2,4-triazol-3-yl)phenyl]-1H-indazole;
6-[4-(4-{[1-(cyclopropylcarbonyl)-3-azetidinyl]methyl}-4H-1,2,4-triazol-3-yl)phenyl]-1H-indole;
6-[4-(4-{[1-(cyclopropylcarbonyl)-3-azetidinyl]methyl}-4H-1,2,4-triazol-3-yl)phenyl]-1,3-benzothiazole;
4-{[(3S)-1-(cyclopropylcarbonyl)-3-pyrrolidinyl]methyl}-3-(3′-methyl-4-biphenylyl)-4H-1,2,4-triazole;
4-{[(3S)-1-(cyclopropylcarbonyl)-3-pyrrolidinyl]methyl}-3-(4′-methyl-4-biphenylyl)-4H-1,2,4-triazole;
3-(3′-chloro-4-biphenylyl)-4-{[(3S)-1-(cyclopropylcarbonyl)-3-pyrrolidinyl]methyl}-4H-1,2,4-triazole;
3-(4′-chloro-4-biphenylyl)-4-{[(3S)-1-(cyclopropylcarbonyl)-3-pyrrolidinyl]methyl}-4H-1,2,4-triazole;
6-[4-(4-{[1-(cyclopropylcarbonyl)-3-azetidinyl]methyl}-4H-1,2,4-triazol-3-yl)-3-fluorophenyl]-1H-indole;
4-{[(3S)-1-(cyclopropylcarbonyl)-3-pyrrolidinyl]methyl}-3-[3-fluoro-4′-(1H-pyrazol-1-yl)-4-biphenylyl]-4H-1,2,4-triazole;
4-{[(3S)-1-(cyclopropylcarbonyl)-3-pyrrolidinyl]methyl}-3-[3-fluoro-3′-(1H-pyrazol-5-yl)-4-biphenylyl]-4H-1,2,4-triazole;
4′-(4-{[(3S)-1-(cyclopropylcarbonyl)-3-pyrrolidinyl]methyl}-4H-1,2,4-triazol-3-yl)-3-biphenylol;
4′-(4-{[(3S)-1-(cyclopropylcarbonyl)-3-pyrrolidinyl]methyl}-4H-1,2,4-triazol-3-yl)-4-biphenylol;
4-{[(3S)-1-(cyclopropylcarbonyl)-3-pyrrolidinyl]methyl}-3-(2′,3,4′-trifluoro-4-biphenylyl)-4H-1,2,4-triazole;
4-{[(3S)-1-(cyclopropylcarbonyl)-3-pyrrolidinyl]methyl}-3-(3′,3,4′-trifluoro-4-biphenylyl)-4H-1,2,4-triazole;
4-{[(3S)-1-(cyclopropylcarbonyl)-3-pyrrolidinyl]methyl}-3-[3,4′-difluoro-3′-(methyl)-4-biphenylyl]-4H-1,2,4-triazole;
4-{[(3S)-1-(cyclopropylcarbonyl)-3-pyrrolidinyl]methyl}-3-[3,4′-difluoro-3′-(methyloxy)-4-biphenylyl]-4H-1,2,4-triazole;
4-{[(3S)-1-(cyclopropylcarbonyl)-3-pyrrolidinyl]methyl}-3-[2′,3-difluoro-4′-(methyloxy)-4-biphenylyl]-4H-1,2,4-triazole;
4-{[(3S)-1-(cyclopropylcarbonyl)-3-pyrrolidinyl]methyl}-3-[3′,3-difluoro-4′-(methyloxy)-4-biphenylyl]-4H-1,2,4-triazole;
6-[4-(4-{[1-(cyclopropylcarbonyl)-3-azetidinyl]methyl}-4H-1,2,4-triazol-3-yl)-3-fluorophenyl]quinoline;
4-{[(3S)-1-(cyclopropylcarbonyl)-3-pyrrolidinyl]methyl}-3-(2′,3-difluoro-4′-methyl-4-biphenylyl)-4H-1,2,4-triazole;
4-{[(3S)-1-(cyclopropylcarbonyl)-3-pyrrolidinyl]methyl}-3-[3-fluoro-4′-methyl-3′-(methyloxy)-4-biphenylyl]-4H-1,2,4-triazole;
4-{[(3S)-1-(cyclopropylcarbonyl)-3-pyrrolidinyl]methyl}-3-[3-fluoro-3′-methyl-4′-(methyloxy)-4-biphenylyl]-4H-1,2,4-triazole;
3-[3′-chloro-3-fluoro-4′-(methyloxy)-4-biphenylyl]-4-{[(3S)-1-(cyclopropylcarbonyl)-3-pyrrolidinyl]methyl}-4H-1,2,4-triazole;
7-[4-(4-{[1-(cyclopropylcarbonyl)-3-azetidinyl]methyl}-4H-1,2,4-triazol-3-yl)-3-fluorophenyl]quinoline;
4′-(4-{[(3S)-1-(cyclopropylcarbonyl)-3-pyrrolidinyl]methyl}-4H-1,2,4-triazol-3-yl)-3′,4-difluoro-3-biphenylol;
4′-(4-{[(3S)-1-(cyclopropylcarbonyl)-3-pyrrolidinyl]methyl}-4H-1,2,4-triazol-3-yl)-3′-fluoro-4-(methyloxy)-3-biphenylol;
4′-(4-{[(3S)-1-(cyclopropylcarbonyl)-3-pyrrolidinyl]methyl}-4H-1,2,4-triazol-3-yl)-3′-fluoro-4-biphenylcarbonitrile;
4-{[(3S)-1-(cyclopropylcarbonyl)-3-pyrrolidinyl]methyl}-3-(3,4′-difluoro-4-biphenylyl)-4H-1,2,4-triazole;
4′-(4-{[(3S)-1-(cyclopropylcarbonyl)-3-pyrrolidinyl]methyl}-4H-1,2,4-triazol-3-yl)-3′-fluoro-N,N-dimethyl-4-biphenylamine;
7-[4-(4-{[(3S)-1-(cyclopropylcarbonyl)-3-pyrrolidinyl]methyl}-4H-1,2,4-triazol-3-yl)-3-fluorophenyl]quinoline;
3-[4-(4-{[(3S)-1-(cyclopropylcarbonyl)-3-pyrrolidinyl]methyl}-4H-1,2,4-triazol-3-yl)-3-fluorophenyl]quinoline; or
4′-(4-{[(3S)-1-(cyclopropylcarbonyl)-3-pyrrolidinyl]methyl}-4H-1,2,4-triazol-3-yl)-3′-fluoro-4-methyl-3-biphenylol;
or a pharmaceutically acceptable salt thereof.

12. A pharmaceutical composition comprising the compound according to claim 1 and a pharmaceutically acceptable carrier.

13. A method of treating cancer which comprises administering to a human in need thereof an effective amount of the compound according to claim 1.

14. The method of claim 13 wherein the cancer is selected from the group consisting of: brain (gliomas), glioblastomas, leukemias, Bannayan-Zonana syndrome, Cowden disease, Lhermitte-Duclos disease, breast, inflammatory breast cancer, Wilm's tumor, Ewing's sarcoma, Rhabdomyosarcoma, ependymoma, medulloblastoma, colon, head and neck, kidney, lung, liver, melanoma, renal, ovarian, pancreatic, prostate, sarcoma, osteosarcoma, and giant cell tumor of bone and thyroid.
